SEQUENCE LISTING (26 sequences)

SEQ ID NO: 1 - DNA, 256 bp, Artificial Sequence
  256-nucleotides sequence (S(DB)) of formula (IV)
cgcaaccgtccgactagctaaacgcaacgtcaacaagtctcgcaagtaacgtccgacccaacccaagttgagttaggagaacccgtttgacgataccgggctccttcgagtcttatcaaagtccaacccgcccaagaaggttccaagcaagagacaaaggcccgctacgaattggtttgagacaaggtagttgccggaacctcaattccgataagttggctcaagacggttggctttgacggaagtcgttaggaac

SEQ ID NO: 2 - DNA, 25 bp, Artificial Sequence
  25-nucleotides [UP] nucleic acid
tatgaggacgaatctcccgcttata

SEQ ID NO: 3 - DNA, 25 bp, Artificial Sequence
  25-nucleotides [DO] nucleic acid
ggtcttgacaaacgtgtgcttgtac

SEQ ID NO: 4 - DNA, 306 bp, Artificial Sequence
  nucleic acid of formula (V)
tatgaggacgaatctcccgcttatacgcaaccgtccgactagctaaacgcaacgtcaacaagtctcgcaagtaacgtccgacccaacccaagttgagttaggagaacccgtttgacgataccgggctccttcgagtcttatcaaagtccaacccgcccaagaaggttccaagcaagagacaaaggcccgctacgaattggtttgagacaaggtagttgccggaacctcaattccgataagttggctcaagacggttggctttgacggaagtcgttaggaacggtcttgacaaacgtgtgcttgtac

SEQ ID NO: 5 - DNA, 3000 bp, Artificial Sequence
  DB-1
ctcttgcgccaacaaacaacaaccgccaacaacaacaaccaacaaccaacgaaagaacgggagccgacgccaattaaggaggcaaagtcctctagctcggaactaaccggaccggtatccgctatttcggccaatcctagtaggtagaaggacgtcttgcgttggctaaggcaacaacaaactcgggttacctatacgcgctcaaattgcgcttggtcggtaagcgccaacggattcaacgaggttagtaacgcgaagggtcggcaacgatctttcccaaggaagctaccaagaacgccctaagaacgttcttataggcgctcctcgccggaaataatctatcgacggcctatttcggcgggaattgggctttccttccttgttcgacgagccaaacttgcggctaaacgtagctattcctccaactagtagcgcgacgtactacgctccaattcttcaagcctacggcaaggttgtttcgtagagattggagcgattgggttacccttgttgggttactttgcgcctaccaagtataggtcgcggacaacaacctcgctaacggtagtacgggaactagacctttaagccctacttgtttcccgcttacgcgaaccttgccaacttgtatcgtcgaccggaaggtttgccttggtacctaaggaccgaaggtcttacggtctttggacccgtcaagagtttcccgtatagctccgggtattaggataaggtagtcgcttggttcgtctttcgggaaaggtcgcaacgaagaaccttacaaagggcgtcggaggctagcttgtctttacaatcccggtcaaacggcgactagtctattgcgctagcaaggcaaggaaacccttgggcgcttaggtacttgttataccgtcgaccgttgccttatccggtttcccgtaaagttgctaccttacggcctttgcttacctcggtagcgctttggacgattacgtaggataggccttgaaacggttggaggaacggttgggcttaccgttacggtctttcgtttctacttggcgccttcgtttactcccttgaccctataacggcttaggtcgcctcgtttctcccgcttgataccgattctacgagagaaaggaggaacgagggcaaggtttgacaagccaacgaggcgtttcaacaatcggtcctagattcgcgttggcctaacaacgttggagcctacgtctaggcaagaaagagcccgggctattacctactccttggaatccgccgtagacaattgggcgatattggtttagacccaaacccttcttaagcaagggacgagtagcctaacctacgcccgaaggaatcttcggttgacaaacgcaaacgttcgcaacaaccttccaatcggaagggctcgttgagggttcaagttcctacgaacgagtttcggacgggtaagccctacctatatacgacgcgtttggccaaaccctaaggtattgggcctttgagaggaagcaaaccgggccttaagcctagtaaagggtccgcgacaagatacaatttacgagtccggaccgccaaacccgtatttctttacgtcctagccttctacggactcgttcctacgaccctcttgctaacgggtctttaaggagagtagtaccggtaagggagataagcggttggttctaccgaggagggaatccttgacggtaccttaccctacctcttgctagttcgttgtagattgccctcttcggcaaccttccctttcccaagggtactatttggcgaacccgttaaaggccgttgagtctaattgccgccttcaaggagagcccaactaaggtatcggacgagatagcggcttctcgagtcgaactcaactagacgagctcgagggttcgggtcaagtcaattgcgtatcaacggacggagggcaaaggaattgaactagacgggttcggcaacgatttgtagttagacccttaccgctactttccctcttccgggaacccaaagaggtagcggttggttgaaaccttgggttacggagcgagcaaggaagttatttgagggcggttgacccttaccgtttgaaagattggcgttgcctcggcgtatttactctttcgtcgtccgatcccaaggcggctaaattaaggcaaaccggacgccggaaattgtagtatcaacgggcgcttaaccggatctttgccttggaattgtcgcgtttgtcctttccgttggttgcttcttggactcccgacttcgtttgctttaggctagaggccaacaaggtagaaggctaagaggagcttcgccttgaccgcttgttgtcaaccggccaataaggcggaaccttattggcaacctcttgttgtccgcctacaatttgggtaaccggtaattcggcgaccaacaaagccctagccaaggatacaagaggcggacaacaacaagaccttgcgtcttacgggaatcaaccctcttgggcttcttcttgacttagccaaccgtcggtttcaagagagacttgaatacggtccgaccgcggagatatagtatcctatacggacgtttcgccctcccgttgtaactccgctagaagatcaatcggagccaaggcgataaggaagggttgaagggttgccttaaggcggtataggcgtcggtaatctcttgccttgtctacgggactccgggacaattaagataagcgccaaagcgtaaaccctcgtccgagaacaaccggtcgtttacaagtcgttaccggacccaaaccgagaagttcgcaaaggccttgtaaccaagagcttgacgcttagtagaccgtcgttctaccaacgtcaagcccgtattggagagcttcggtttaggaacgcctccttacttggaggtttacgcgagtaacgtaggacaatcgcgggaacgttgagagaaggaatcgccaaacaacgagcgaacgataaggacttgcttgacaagagcaacgaaggcaagccgggttagtcaacgcggagg

SEQ ID NO: 6 - DNA, 3000 bp, Artificial Sequence
  DB-2
ggatcggagcgttggtttgtccgctacccgtttaacgggtaacgagttgaaagaacgccgacggtataaggcaagttctacgggaccgattcgttggtagggacttagcttccgggttcttggtattacttcggcgaaacccgactttctagggtcctaaccgttcccaaggcctttggaacgttgggtagattgaagggttaagaacgcgggactaattacggtccgagggcgggattaagttgtaaccttccgcctagttaagcccgtctatagagcggccgactatacctccgaaatactaaggcgcgttgagcgtattacttgagtttcccggcaaagcctttgcgtagtcggtttagagccgcgaccaatagaaggttacttggcctaggtaaaggagggagggtatttaagcgctcccgtttcgctccctaatcgcaattggtcaacgactaattcgggacggctaaactcaaaccgtcgtccctagcgacgcttaacctacgacgacaataggccgtattgcgttgtcgcgaacaaagaggttacgcctcttagactactagacgccgtctttaagcgacggcaaattggaccgtagacgactacaagagcgggagtagacttgaagggttagagcgcaaccgtccgactagctaaacgcaacgtcaacaagtctcgcaagtaacgtccgacccaacccaagttgagttaggagaacccgtttgacgataccgggctccttcgagtcttatcaaagtccaacccgcccaagaaggttccaagcaagagacaaaggcccgctacgaattggtttgagacaaggtagttgccggaacctcaattccgataagttggctcaagacggttggctttgacggaagtcgttaggaaccgggtactcaacccttgaggtagtttctaggctcccaagcggtatttgacgcctttcggtctaagaactccttcggttcgtcgagccgtttagttacttctttggcctagcgggatattggaagctcgttaaggccgataccgagaaggcgaggaaagggcaaacctacgttggaaggtcaagttaggttgaccgattgacgcttatatcctctcccgacgtagggtacggcttgaggtcgataccttcccaatcgtagaggaaccgtcggactaacaacgagttaaggcgttcgccaagcctcttaggaaggaggaaagcttgcgctctatccggttagattgtccgttagccaagggataggcaagttgctccttacctcaaccctacgaagaggaaaggcctaccttacggtttcggtaacgggaagcaacggacctagttgactactcgctctccgttccaactacctccctccaattctcggtctagaacgttcaacgggccggagatctcttgtaaaccctaattcgagattgggcaaccggaacttgaattgcccgggtaggaagcgtttgtcgagaaaggtcggaggttgagggtattcaagcttgagcgggaaccttaggacgaaatcgtcgccctaagttgactctccgaggttatcaagggaagttggcgaacttccctaactatcctacgacccgtccaatacgaaatcggagttcccaacaattgccgacgtaagatcggttgtcgaagcgtccctctctctagtctcctatagctcctcgccttctatccgttaactcctcgacttgaccgtacgcttaagggcaagtcgctatacccgggtagacttaacgggtcttaacgttggtcggcctcaatttctattcgcaaaccggtaacgagttgttccgcgttctctccttcgtcctactaaggcttggcgaacgaaactaacgccgcgattcttggaacttgcgaagagtaccaattacgcgggaactcaaggcgaataggcaagcgttgatacccgtcgtagctaagtttcgaaggttgcgcaactcgttgttggactagccgctatttccgtttcggtcgggactttcccaaacttggaaagggaatctcgagggcgaagtccaaactaagggacttgcggaccaacaaagggtcgcggtaatctttcctcttatcaacggtcccttatcctagccttagacccttctcccaagggcaactcaatcttcgttccggttccgacttcgatctagcgtatcttccgtaaccggcgcttgccctaactagactattggcaagcggttgagaatttcccgacgcgtacccgtctatttaagaaggaggagccggttgcgaaggaaaggagggaaacgtaagggtcaagtagagtctacaacggaccaaactcgggaccaagtttcgctacctttcttcgaccaaaggcggtattgttggtaggccttgttgattgagaaacccggacctcggtaggactttacgagcgttgcttagtccgccgtaattagttagaagctaccgggcgggagattctaaggaagagcgggtttagaagggtaacgacgttgagagatacaagggtctaccgacctccaacggcggttggtttcaaagccgccgtacttgttggtaaacttgctcaaggacgggccttagtaccctatagaagcaatccttagaggacccggaagaacgcaatagacaaccgaaacgaggcgttaggtactaaccgggtcgacgctcgttatcaacttggagcaacgaacggcaaacaagacccgaacggtccgaattaagcttgacggaacgggttcccgttgataacgtccgtctcaatagttgcgtctagttggcgccttggtttacaaaggcgaaaggttgctatctttgccgggtacccttaattcggaacctccttggtaatcgcctctacccggattcgtagtagccctccttaatttgcccgccaactcttgctacctccttaaactcaagcggccgtccgaccaaaccaattagcaagcgctacgaacgtttgtaaggcgttgccgtcaaga

SEQ ID NO: 7 - DNA, 3000 bp, Artificial Sequence
  DB-3
aattgagattggaccaagaaagcggaaggaggaatcgggtagttgtaagagccgaaagtaccgaagaagcccaagttggtcaagggctacaaggcaactagagtcttagccgggtcaaaggccaaacctcgacttgttggtagtctaacgggcaattgattggtaccggtcgctagttagggagcttcccaaattgaggctaccctaagcaaacgcggtaagctcttgggttgggacaatcggactttgggcaacgacttagtatcgaccggatagtacgtaacgtcttggcttcgggagtaggttacgaaactacccggtagctactttcgtaggtttcgtctccggtccggtttcgtttcttgtattcccttggcggtcgttgccaaccaatcttggccaacccgtataaatccgctcgatttgacctttcggcctaagttgtacctaggacgttagaacgacctcgcgtagtaaaggacgggaaggtccaacaacgcttacggttgagctcgttgcccttatagaacgcggttgttgttcccaattcaacctcgcctaacctttgtccggttcaacggaaatcgggacttacgggaacggttacccaagaaccgtccaaaccgataaaccgttagttacctcggccgagtcaattatccgaggacggactattccgggtcgctcttggaatacgccgaagttgttacggcggagttctagagtaaagcctaagggctaagtccctctaccgaaccgacgatcctaaacgcgtttcggaaagttggcgaaactccaaacctctcaaagagccgtcgaagaggcaaaccaagtagcttaaggtcggccaataggaccttcgctagtccaagggaaaccggagattaaggaccttctaacccgctaccaacaacaacaaggtaccgaaacggaacccttccgtcaaacttgaacgcaaggttagggagggtccgattaacggcaacgattacttgcttgtaaggcggagcgtctagggactctatagttggacgagcctccgcaaacaagagctattattaccggaccgggtttggtcctcaatcgacgtacggaggtaaggaattgcccttggagcgggaaaccgttagtttacccgtacgggtttcgttccttgcctagcaaataaggtcgcggttatacgccgttgcttagaagccaacgaccaattgggtagggcgacgggacgatagtttgtccgttgttgctatcctctcgactatcccgttaacgggctaactataccgttgacggacgaaggatccgtaaagggccgttgttgactcaagcggcaatttccgaagcgcaagtaacgagaaggcgtcaacctacttgaaacccgaagaggagccgttgacaacccaaagaaggaattgccggcccaagcttcgttcgcttgttaacttgtagtccgacttggcgaccttgtatccgaataactcccgacgaaggtcgttcgatactcgagttcggccgggattagttcctctcaaggaaagggcaacgaactagccaaagtacgcaaggtaccctagaattggcaaggaacgggtttgctaccgacgcctaattggccgacaataggaggctaaacgttgttgccgtccgggtttattcgttccaaggctttggcctttattgcttcgctcgcgaacaatccgttctataacgcaacgcctattgggaaggcccggtttgtttcgccaatttgccaagtcggtcctcttgatcgaacgctaactatcggagacgttcgtccgccttaaggaacgcttgttaaggattccgcggaaggtatatccaagaaaggcccgacgtcttgcctaacccttatcctaccttccgacaattcctccttggcttgacgattacgagaagattcgagccgtccgaggtactaatctaaaccgggcgagtcgcaaagtttaacgccttaccctacccggaaatttcaaccaaccgaagcgcgtcgaaccttaaatctttccggcccttgctagtcggtacgttcctcctctcctttacctcttaccgcttaggctagtacgggacgttggaagtaaaggacgcgggcctacaaacgctattacccaagttggactccgggcaacttagaagggtcgttaaggcgctagagcggaagaaccaagttggatagcggacccaaccaagattattcgaggaaagcgcctagtccgttcctccaaggcgaaatttggaggaccgacttgattcggagcaagaagttacaagggccgacaagacaattcgctcaacctcgaacgtcctaaccgtctctcttgctactctaccaaccgatagggctccctaatatcgagaggtttgcctctcgtttcctcttaaccggaagggttacaaggcttgaaccgataacccaagagctaccggcgtaccgttccgaataaattaccgggatcggacgtccttcgaattgtccaactccggcctatttgttggcctccgtaaacgacgaattgcgtccggattgactttcccggagatcaagtccaagacggcaatacgttgggcctcaacttaggtcgccgagctatctttcccgactaacttgattggttgcgcgtcgatagtatccaattggactttcggcctagggacgttccgtataccaaggcttggacccaacttattgagcctaccgcttcgtaggctaaccttctaggccggcttaagacttaagaacgccgacgcgacaagtttacccttgcgctctctaaggttgcggtaagaggaattgagcctaaacccggcgctataacgacccaagtctaactacccaattccggcttgcgttgctcgcaatcctaactaattgagccgaggaaccaagattggccgacccttccggtttgaaaccaaggagctaacgttccaacttggcccaaagttgaaactcgggagggccgtagggaagtagtatct

SEQ ID NO: 8 - DNA, 3000 bp, Artificial Sequence
  DB-4
cgttgttgcgataggtattgaagcgagggtagaagggctattagttacgggcgaacgggatttggaccctcctttacttggcgctctagaatcaagggccgtaccttctttgtccttcggaaccttgcttgtcgtcaactcgctacttagatctcggcgacggaccaactataagtaagccctaaggcgtttgggtcgcaatataacggcttaggcctttcgccctttggacctacaagcaagagagagggatcaaacggttgggatttcggatccggacaattgtatccctaaccaacgacgacgttgttgcgcgttagggtcctttgaacgcgaagggaaaggattggcgaccttgggctaattactttcgcgcgtaacctacttcgaccttgagtacgccgaaattgctcggaccttctacgtatcgtaacaaccgctctacgacggccgtttcaaacaacgttggctaccgtagtccgtaggtacgagtaggaggaagggaggttagggtaattcttccggacgaaatcaagtagcgttgggccttagtcccgattcgaaggagctacttgtccgctacctccaacttagctccaacctaacccgggattggcaaataacgtaggccaaacgtcttgagttgacccaaacgtagttgggacggaacccttcggctttagaaggaattagcccgctacaaggccaaacaacgacgggcgtttacgtagttgttccgttgacgttcaaagggacttggtcgccaatctactcgcggaaacaacttgcggtcggtaagtaagcgacggtaagaaagtcctcttccctagttcaagcggcctaattgaccaacgaacgtacaattcgacgctagggagatatagcgcctagggaaagggcgggataattccgattccgtacggcaagttcgtcttgttcccgggcttcttagtttacggtcctacgcgttagagtcgttgcgagtcggttccttattcggtttccgccaagagggtctataggactagtacaacccggtcggcccttctttgcttaaaggctctcctccttggcgcgtaactttgtcaagcgttgcgaagtaggtcaagaggaaaggcaacgctagcgtaactccgtccaaacgttacttgtcggtttgacggagaaaccgtaacgacctccttgggtaagttcgatcttggctagcccttgctagtcgtctctcctcctttacgtaaggacaagcgtagcgctaactaggctacctctctccaaaccaaggaattgggtagcgaggtatccaacctctcaagtcggaataaggcccgttcccgtattatccaagacgtctaaccggactaccaacccgaactagggcggaattgacgtaatattccgtcccgccttaatcgactcgtacgggatacggttaagtcttgctcgggtaatcttggcggttccctaactttcttcgccgtagaagtacgggtcgaaggcgagggatttgggtattggaggttacgctagccgattgtcaaggccgcttatagattggcctacttaggctagattgcggaactcgaatcgacgtttcggtagccgaagaaagggaacgcgcctataagtacctcgaaacccttcgttgagtagcctctcgaggataagcggttattgcgtcgggaagagttctagttacccgtacgccgtatccctatctttaacgggtaagccttgaccgcggtagatagtagggatcgtactcaaagggaagcgctcctcgttagaaaccgacgaagcggcttgctatacaacgccgtactttaggcgatcccgtttcaaacccgcaaagcaagtctcgcttagcctaatctttggcttggccaacgtaacggctacttgggcgtctttactcgcctaaattgccggcctctctatctcaacgggagcttggaattgggtaacttgctaggcaaaccctagaggttcaacaaggtccgaccaacggagggtatcaagttgagtaggtaggcaaagaagggcgaacggttaacggaacaagagccggtaagcgtcttggtatcttcgttgcctcaacgtcggtctagttgactaagtaccgtcgaggcgtttgcccttgagaacggttagaacctctcgtcgagtagaggtttaaggtcggaaagaaatcggtccgcggtatactctactaccgaaaggtcgtcccggtagctagctaaacttggaaactcgggcccaatctccaagacaagtacaaggcctagcctcggcttaaaccaacctcggagttgcttaggctacttcgtcaaggtaggctaatcgggcgttgattaaacgctacggtaccgaaagtaccggacgtaacctactccgtttacgccctagtcgaaagggtaacgcttgggagttcgttgttgcgcccttacgtcaaagacggataaggagaagccctttcggtagttcgcctaagttgaactccgttgcctacgactaaccaaccaaggttgtccgtctcttaaggctcggaacttcaactcgggagacttgttcgaacgatattcgttgcgggttcggctttagaacgaaaccgaagggacggaacgtttaaaccctcctccttctttccgccaaagtcgaaggactcgctttaacgcccaaataaccgccaaggatcggcgcgtagtattagttcgtcttgccctatttgggaacttccctccgcaacctttacgctaccttacttggacgagagccaagtagagccaacggttacctcttagggcccggtttgtaatataaggcctcgctaaggaagagcgcgaaccgcttaaatctccttagcggcctcttcttattaccggcgactacgcggaagaggacaaattcccaactcgttcaattgggcccgactttggtccaacttagttggagtctagacctaggtattggcccggttaaccgggttagggttgtctagggaga

SEQ ID NO: 9 - DNA, 3000 bp, Artificial Sequence
  DB-5
accgaaaccaattggtttctagagcggtagggtagctagggttaacttacccgtcaacggccgttagcccgttaactacaaaccgttgggacctttgggcaacttgttgctacgactttaagctcgaacccttcgagagagcaagtacctagctcggaagtaacggcctagatttactcctcgccgaagtaggaagatcccggtcgacgcgttgtttctaggtttgggttgctccttgggacctttgagtaacgagttcgaaccggaccaatacgttgccgtacctctttaatagtcttgaccgcctcggttggcttagacggtcggttagcaatatccgtcctctagaagggtacccgaaacttgcgtagtaacgcctaaggacgtcctagacctaaccgaaagaccctaggagcaaggtacctaaggtcctatagtccgagccaaaggtcgtagttgacttagaggccgggatcaagagggaagggacggatcaaactacttgaaggccttcggccgaattgacctcaaatattcccgggaccgttgggaaccttgctttccgagtctccgctagtcgttgaacttgagggatcttctactccgggaaaccgtcgcaaaccgacaaggcaataggacttctaatccgtcctagggagccttcaagagtcgacgagtcttggtaattcttggacgtttacgcggtacgagcgttcgcttatcaaggcctccgatactaacttaaagggctcaaccggttgtcctaatccggagtactaagtacgggcaaactcggtaggacccgttgaaagaacaaagcgacccgaaagacttgcgttattccggctttcccttctcaagtcaaaccgtccgcttctttggaggttgttgaaggcctccctcaactcctaaccaaacaagaccaaggagcgaggagagaaattgcgaagcgaccctaaccgagaaacttgtacgggtcaatcgcccaaccaactattacccaaccgtcctcctacttggctacaagggttagcgtcaagatttggcgcttaggaaacctcttgctagagcaaagaagctcccggaagatccaaaggacggtagggttctagcttcaaatcgcgcttgacctaataagccgacggagccctcggtaaagtcaattcgggtagcttagaagccgcaaggagattacccttcgttagtccctccttcccgattcgagaggattacaacggaattgggaccgaagtaggccggaagttacggagttcgtcgtttcaaaccctcccaaacttacgggacgtattgtagggacccgggattccaaccctaattgttactcccggcgttgagttagagccttagacaaggtcgccaaacggaccctaactaagacgagttagcccgccaactaataccctagtaacccttcggtaaggtcgccgactccgatatcaaacgcgttagaaaggcgacggcaattggtacggtctacgttgtcttaggacggttcgggaacaacgaggaaagacaataggcgctaagccgttgagtcccaacgtaagagaggagcaagacttgaaggcttggtcgggtctaaggcaaaccctttagcgattgcccgaggaacggtagttgatccgtaggttggagtctccgattaatccgttccggtttctccctcggttcgtcgagataagggcgattagaggtttagcgccgtaagctaatccgaacgactacttccaattgctcggcggttacaagcttgctcgttacgacggcaagatagtcgagggtttacgtataccgggagctacgccttcttggtttccaaatccggtcgctaagggtacggtttaatccggtagccgacttggtaaccttattcgggaacgacaagcgcaagcgatcccaaacaaaccgtaactcgggtaatcaacaagttgccgtccgctttacctcgttgtaagtcgggttagggcccaatctaccaaccaagggttaacctcttctattggagacggccgataagctttgcggccctcctaaccaattgagtacgttgcgttcttcccgcccttatacgtacctttcccttcttgtacgccgaccgttgacgaagtacctctagcgctagctatccgaattgttacgggctataggtcgtaaccgggatcgaagctcctaattccaagaccctttggcaacgattctaccaacggacgaggagctacgttcttacaagaacgcgcgaggaaggttaggccttaaacaaggttcggtacgattcccgggattctagcggtttagctccaaggcccttagagggtcctttaacgtttaccaatccggacgtcggacaactattgcgattggacccaaccaactcttggcaattggaaactaagggccgacaataggcctacgggtagtaggagtaagacggtttcgttggcggtaactagacgcgagttgacaacccaaacaaggtagtctacgaagcggactcttcttccggagtcccgactagattacgaaggacaaagtcgcaagcctcaagccttgggttggagttctaaggagcccgtataaacgcaaaggaggaaacgacttgaagccgaggaaaggcggaagtaatacttgaccaaccgcaagtcgctctattgtcgctagctagaaggtcctaccgagggcgtttgaattagacgtttggcggtcaagcaaacgggacgggagaaacctcttgcgttctctagtacttccttgggagtcgaacgaacaagcttgtagaacctacggtagcggccttagggtattacgttgttgacgaagggcggtactcccttgttaacttgaaggcccggttggcttctaccaatcaagggtacgttgggtcttgacggctaggttaatcgcgctccaattggcgcgtcctaatatagctctattgggaggacggttacgtattcggcctaaggttgggttccttgaaca

SEQ ID NO: 10 - DNA, 3000 bp, Artificial Sequence
  DB-6
ccgaaggccgggagaagcaattggagttgaccctatctccgcaagacctttgcgcaaccttgtaaggacgttgaaacggttgtacccaaacaacggccggttgatctaagggtaatccaagggcttacgggagcaacaagctagttcggactagagggtcctctagcgataaagctaacgttcgagaacgccgccgctaattgttgttgtcaagctaccgaggcttcgcaatacgatcgaagctagtccgacggctcaaaggaaagccgtaatcgtcgcccaactaccaatcgaaccttgaggtacccaagaggtcaataccctatcctaggcgaacgaaggatacaagggacccaatagcaaggttggatagtcgcccttggtattgggccaagaagaggcgatatcgtaaccgttgatccgttgagttacttcgaccgctctaggttcttacggtcggtactcgcctaaagcggcttgtttcgaccttaaaccgaacgggcaagacgaattgaggaacaaggctcccaaccaaggttgaacgttagcggcaattgatccaaccgtcgtaaccttgttgtagggtccgtttaaccgctaagcctaaggaagccgagtcctaaataaaccaagccgcggactaggcaacgggttagttgtccgataattaccgagcgaccggaccgcaaatacaagttagtagaggcggacgtctaacgttgctcgaattggatcgactcgcaagaccctagttgggacttcccttgggattacctcttcgccttctagcaaggttgttgcggacggtagttaggtcgacttggtcgttcttgaccttcttcccgtccctaagctagggtaaccaaaggttcgcgattcgtccgggtaaatttgcgtagcgcgttgcttctaataaccggatcctccggatacccaatcgttggttaactctcgtccgattaggcgtcggcgtttacgttaagggttagaagaggcgggcaaaggaacttaacaacgttacccggaaagcgaccaaaccgaagtcgatcctcttgacgagaggagaaggaatttccaaagagcgggcttcaaacggctctagcggcttcgtctaaaccttcgcaaccaacttcccaagacgacttggaacgcgtagagtaaggccgctttgttgttgggatcttcttccaatccggatatccggtcctaccgaagaaggtattggtaagggatcgcctccgttgaccgattacttacttgatcgcggccgttagcaagtccggtatacgcctcaatagtttacgcaagcggaggaagcttggttgggtaaccgaagggaagctccctacttagacgggtttggagtttacgtccaaacgctcgcctcttattaccggaagccctcgatctttggaccgcgtaggacaactacctacgtcgctcttaaattcgaccgacaacgttcgtaaggcctacggcttccttggttgttacggcgttcaatagcaagcgttagcgaaggccgggagtaataataggagggagtcaagagagattatcggtcggagccaagttgagtcggacccaagttaaaccgtcgtcgtacggttaatcctctaaccggtaaacctccgttcgttgggtcgatccaaacctttctctcctaccgaccgcttaagcctagtcgaggtaccaagctaagtagtcaacggacgtaacttcccgtcgttacgcttggactacttcttaagcggcgacaaggattggacgtccaagtacccaatctcgagtagctcaattaccgggccaactacccaagactaaggcgggttgttcgagctaaccaaggactcccgacgtaattgtacccgccgatcgtaaattcgcccaataagcggattgggtttgtcggactccgacgttaacgaacccaataagggatagttaagggcccgacgaagttaaagccggacggctaaggtacaattgttacggcccggttcctttgtccgtttctacctcaaccttgccgcgaacgactcgtttaaccgaaggctagaaactttggcgaaacggtacttacgagggacctagctttgtaagcggttatccgcgcggacttatttggaggtcgttgggcctaacctcttggttgtagacccgataccgtcggcttcaattggaagatcgaaggctccttagtatcgggaagggtagttcctacgcgttaggaaaggctcttaaacgcccttgagcaagccgtcaacgtttagtcgttgttggacccgaggttgtaggtaagtaggatacgactcgggtccgcgaagaaatataggtcggcaacttagagtcccgagctttagggaacaacaaacctcggctattcctacgcggaaacgctacctaaagaagggcaacgacaagcgcgaacggtataacgggtacgactactcccgacttaggttaccttaacgggcaaccgaacgcttagctcaaacgttcggtcccaaccttcaattgtcgggtccctctagggttacaatttgctccgcctctagggaggtttcgctatagcaagttcgttggtaggcttgacgggtttggttgcgaggcaatataggaggtattgggtctttaggacgagcgagtttggccgcaatcgaatttgacgcctagggcgaaccggtttaaccgaagcaactattggttgggttcaagggttgggtctaccggaaatcgagttgggtcgtactccggagttctcaagccaacttaggcaactcaaacgattcgtccgctaactccgacttggttgtcgtataagcgctctcctttggagtctttaacgtccgggtttacgagactacgcgattgcctcctaaagcctttaaccggtagccgggcttaagcccgaaagagctttgaaacgcggatatccgcttgttgtcaattgtcgaccggccttagctaagggacggagggctagtttatacgggtcgg

SEQ ID NO: 11 - DNA, 344 bp, Artificial Sequence
  DB-7
ggttcccggaacttcaatcgttccgctcctttaggttgaaggcgtttacggcgggtaaccgtttagggttagcaaggcaacctcgaggaaataggtcgggtattgagggaagtactagaagcttcccggagggttagaaggagggtaacggttaacgggacgaggaccaatttgtcggttgtcaacggttagttcgggtcaaaggaaaggacggatactttgctcggccgacctagaagttgagaagagaggtccggaggttgggtttcaattaggttgccgtttagggcctagggtaacaaggtttggtttcgagccgcttgactacggatttgaccaaacca

SEQ ID NO: 12 - DNA, 26 bp, Artificial Sequence
  UP-1
tatgaggacgaatctcccgcttatac

SEQ ID NO: 13 - DNA, 26 bp, Artificial Sequence
  UP-2
gtttatcgggcgtggtgctcgcatag

SEQ ID NO: 14 - DNA, 26 bp, Artificial Sequence
  UP-3
tagtagttcagacgccgttaagcgcc

SEQ ID NO: 15 - DNA, 26 bp, Artificial Sequence
  UP-4
ggggttccgttttacattccaggaaa

SEQ ID NO: 16 - DNA, 26 bp, Artificial Sequence
  UP-5
ctgacgtgtgaggcgctagagcatag

SEQ ID NO: 17 - DNA, 26 bp, Artificial Sequence
  UP-6
gaggtctttcatgcgtatagtcacat

SEQ ID NO: 18 - DNA, 26 bp, Artificial Sequence
  UP-7
ggtaactgcgcatagttggctctata

SEQ ID NO: 19 - DNA, 26 bp, Artificial Sequence
  DO-1
aggtcttgacaaacgtgtgcttgtac

SEQ ID NO: 20 - DNA, 26 bp, Artificial Sequence
  DO-2
accgatgttgacggactaatcctgac

SEQ ID NO: 21 - DNA, 26 bp, Artificial Sequence
  DO-3
accgtacctagatacactcaatttgt

SEQ ID NO: 22 - DNA, 26 bp, Artificial Sequence
  DO-4
atatcccgtgaagcttgagtggaatc

SEQ ID NO: 23 - DNA, 26 bp, Artificial Sequence
  DO-5
aggtatggcacgcctaatctggacac

SEQ ID NO: 24 - DNA, 26 bp, Artificial Sequence
  DO-6
agattcaatatgtgtcgtctatcctc

SEQ ID NO: 25 - DNA, 26 bp, Artificial Sequence
  DO-7
agcgtttaaggtcacatcgcatgaat

SEQ ID NO: 26 - DNA, 18732 bp, Artificial Sequence
  Full nucleotide sequence of the DNA DRIVE according to example 3
tatgaggacgaatctcccgcttatacctcttgcgccaacaaacaacaaccgccaacaacaacaaccaacaaccaacgaaagaacgggagccgacgccaattaaggaggcaaagtcctctagctcggaactaaccggaccggtatccgctatttcggccaatcctagtaggtagaaggacgtcttgcgttggctaaggcaacaacaaactcgggttacctatacgcgctcaaattgcgcttggtcggtaagcgccaacggattcaacgaggttagtaacgcgaagggtcggcaacgatctttcccaaggaagctaccaagaacgccctaagaacgttcttataggcgctcctcgccggaaataatctatcgacggcctatttcggcgggaattgggctttccttccttgttcgacgagccaaacttgcggctaaacgtagctattcctccaactagtagcgcgacgtactacgctccaattcttcaagcctacggcaaggttgtttcgtagagattggagcgattgggttacccttgttgggttactttgcgcctaccaagtataggtcgcggacaacaacctcgctaacggtagtacgggaactagacctttaagccctacttgtttcccgcttacgcgaaccttgccaacttgtatcgtcgaccggaaggtttgccttggtacctaaggaccgaaggtcttacggtctttggacccgtcaagagtttcccgtatagctccgggtattaggataaggtagtcgcttggttcgtctttcgggaaaggtcgcaacgaagaaccttacaaagggcgtcggaggctagcttgtctttacaatcccggtcaaacggcgactagtctattgcgctagcaaggcaaggaaacccttgggcgcttaggtacttgttataccgtcgaccgttgccttatccggtttcccgtaaagttgctaccttacggcctttgcttacctcggtagcgctttggacgattacgtaggataggccttgaaacggttggaggaacggttgggcttaccgttacggtctttcgtttctacttggcgccttcgtttactcccttgaccctataacggcttaggtcgcctcgtttctcccgcttgataccgattctacgagagaaaggaggaacgagggcaaggtttgacaagccaacgaggcgtttcaacaatcggtcctagattcgcgttggcctaacaacgttggagcctacgtctaggcaagaaagagcccgggctattacctactccttggaatccgccgtagacaattgggcgatattggtttagacccaaacccttcttaagcaagggacgagtagcctaacctacgcccgaaggaatcttcggttgacaaacgcaaacgttcgcaacaaccttccaatcggaagggctcgttgagggttcaagttcctacgaacgagtttcggacgggtaagccctacctatatacgacgcgtttggccaaaccctaaggtattgggcctttgagaggaagcaaaccgggccttaagcctagtaaagggtccgcgacaagatacaatttacgagtccggaccgccaaacccgtatttctttacgtcctagccttctacggactcgttcctacgaccctcttgctaacgggtctttaaggagagtagtaccggtaagggagataagcggttggttctaccgaggagggaatccttgacggtaccttaccctacctcttgctagttcgttgtagattgccctcttcggcaaccttccctttcccaagggtactatttggcgaacccgttaaaggccgttgagtctaattgccgccttcaaggagagcccaactaaggtatcggacgagatagcggcttctcgagtcgaactcaactagacgagctcgagggttcgggtcaagtcaattgcgtatcaacggacggagggcaaaggaattgaactagacgggttcggcaacgatttgtagttagacccttaccgctactttccctcttccgggaacccaaagaggtagcggttggttgaaaccttgggttacggagcgagcaaggaagttatttgagggcggttgacccttaccgtttgaaagattggcgttgcctcggcgtatttactctttcgtcgtccgatcccaaggcggctaaattaaggcaaaccggacgccggaaattgtagtatcaacgggcgcttaaccggatctttgccttggaattgtcgcgtttgtcctttccgttggttgcttcttggactcccgacttcgtttgctttaggctagaggccaacaaggtagaaggctaagaggagcttcgccttgaccgcttgttgtcaaccggccaataaggcggaaccttattggcaacctcttgttgtccgcctacaatttgggtaaccggtaattcggcgaccaacaaagccctagccaaggatacaagaggcggacaacaacaagaccttgcgtcttacgggaatcaaccctcttgggcttcttcttgacttagccaaccgtcggtttcaagagagacttgaatacggtccgaccgcggagatatagtatcctatacggacgtttcgccctcccgttgtaactccgctagaagatcaatcggagccaaggcgataaggaagggttgaagggttgccttaaggcggtataggcgtcggtaatctcttgccttgtctacgggactccgggacaattaagataagcgccaaagcgtaaaccctcgtccgagaacaaccggtcgtttacaagtcgttaccggacccaaaccgagaagttcgcaaaggccttgtaaccaagagcttgacgcttagtagaccgtcgttctaccaacgtcaagcccgtattggagagcttcggtttaggaacgcctccttacttggaggtttacgcgagtaacgtaggacaatcgcgggaacgttgagagaaggaatcgccaaacaacgagcgaacgataaggacttgcttgacaagagcaacgaaggcaagccgggttagtcaacgcggaggaggtcttgacaaacgtgtgcttgtacaagggtttatcgggcgtggtgctcgcatagggatcggagcgttggtttgtccgctacccgtttaacgggtaacgagttgaaagaacgccgacggtataaggcaagttctacgggaccgattcgttggtagggacttagcttccgggttcttggtattacttcggcgaaacccgactttctagggtcctaaccgttcccaaggcctttggaacgttgggtagattgaagggttaagaacgcgggactaattacggtccgagggcgggattaagttgtaaccttccgcctagttaagcccgtctatagagcggccgactatacctccgaaatactaaggcgcgttgagcgtattacttgagtttcccggcaaagcctttgcgtagtcggtttagagccgcgaccaatagaaggttacttggcctaggtaaaggagggagggtatttaagcgctcccgtttcgctccctaatcgcaattggtcaacgactaattcgggacggctaaactcaaaccgtcgtccctagcgacgcttaacctacgacgacaataggccgtattgcgttgtcgcgaacaaagaggttacgcctcttagactactagacgccgtctttaagcgacggcaaattggaccgtagacgactacaagagcgggagtagacttgaagggttagagcgcaaccgtccgactagctaaacgcaacgtcaacaagtctcgcaagtaacgtccgacccaacccaagttgagttaggagaacccgtttgacgataccgggctccttcgagtcttatcaaagtccaacccgcccaagaaggttccaagcaagagacaaaggcccgctacgaattggtttgagacaaggtagttgccggaacctcaattccgataagttggctcaagacggttggctttgacggaagtcgttaggaaccgggtactcaacccttgaggtagtttctaggctcccaagcggtatttgacgcctttcggtctaagaactccttcggttcgtcgagccgtttagttacttctttggcctagcgggatattggaagctcgttaaggccgataccgagaaggcgaggaaagggcaaacctacgttggaaggtcaagttaggttgaccgattgacgcttatatcctctcccgacgtagggtacggcttgaggtcgataccttcccaatcgtagaggaaccgtcggactaacaacgagttaaggcgttcgccaagcctcttaggaaggaggaaagcttgcgctctatccggttagattgtccgttagccaagggataggcaagttgctccttacctcaaccctacgaagaggaaaggcctaccttacggtttcggtaacgggaagcaacggacctagttgactactcgctctccgttccaactacctccctccaattctcggtctagaacgttcaacgggccggagatctcttgtaaaccctaattcgagattgggcaaccggaacttgaattgcccgggtaggaagcgtttgtcgagaaaggtcggaggttgagggtattcaagcttgagcgggaaccttaggacgaaatcgtcgccctaagttgactctccgaggttatcaagggaagttggcgaacttccctaactatcctacgacccgtccaatacgaaatcggagttcccaacaattgccgacgtaagatcggttgtcgaagcgtccctctctctagtctcctatagctcctcgccttctatccgttaactcctcgacttgaccgtacgcttaagggcaagtcgctatacccgggtagacttaacgggtcttaacgttggtcggcctcaatttctattcgcaaaccggtaacgagttgttccgcgttctctccttcgtcctactaaggcttggcgaacgaaactaacgccgcgattcttggaacttgcgaagagtaccaattacgcgggaactcaaggcgaataggcaagcgttgatacccgtcgtagctaagtttcgaaggttgcgcaactcgttgttggactagccgctatttccgtttcggtcgggactttcccaaacttggaaagggaatctcgagggcgaagtccaaactaagggacttgcggaccaacaaagggtcgcggtaatctttcctcttatcaacggtcccttatcctagccttagacccttctcccaagggcaactcaatcttcgttccggttccgacttcgatctagcgtatcttccgtaaccggcgcttgccctaactagactattggcaagcggttgagaatttcccgacgcgtacccgtctatttaagaaggaggagccggttgcgaaggaaaggagggaaacgtaagggtcaagtagagtctacaacggaccaaactcgggaccaagtttcgctacctttcttcgaccaaaggcggtattgttggtaggccttgttgattgagaaacccggacctcggtaggactttacgagcgttgcttagtccgccgtaattagttagaagctaccgggcgggagattctaaggaagagcgggtttagaagggtaacgacgttgagagatacaagggtctaccgacctccaacggcggttggtttcaaagccgccgtacttgttggtaaacttgctcaaggacgggccttagtaccctatagaagcaatccttagaggacccggaagaacgcaatagacaaccgaaacgaggcgttaggtactaaccgggtcgacgctcgttatcaacttggagcaacgaacggcaaacaagacccgaacggtccgaattaagcttgacggaacgggttcccgttgataacgtccgtctcaatagttgcgtctagttggcgccttggtttacaaaggcgaaaggttgctatctttgccgggtacccttaattcggaacctccttggtaatcgcctctacccggattcgtagtagccctccttaatttgcccgccaactcttgctacctccttaaactcaagcggccgtccgaccaaaccaattagcaagcgctacgaacgtttgtaaggcgttgccgtcaagaaccgatgttgacggactaatcctgacatcatagtagttcagacgccgttaagcgccaattgagattggaccaagaaagcggaaggaggaatcgggtagttgtaagagccgaaagtaccgaagaagcccaagttggtcaagggctacaaggcaactagagtcttagccgggtcaaaggccaaacctcgacttgttggtagtctaacgggcaattgattggtaccggtcgctagttagggagcttcccaaattgaggctaccctaagcaaacgcggtaagctcttgggttgggacaatcggactttgggcaacgacttagtatcgaccggatagtacgtaacgtcttggcttcgggagtaggttacgaaactacccggtagctactttcgtaggtttcgtctccggtccggtttcgtttcttgtattcccttggcggtcgttgccaaccaatcttggccaacccgtataaatccgctcgatttgacctttcggcctaagttgtacctaggacgttagaacgacctcgcgtagtaaaggacgggaaggtccaacaacgcttacggttgagctcgttgcccttatagaacgcggttgttgttcccaattcaacctcgcctaacctttgtccggttcaacggaaatcgggacttacgggaacggttacccaagaaccgtccaaaccgataaaccgttagttacctcggccgagtcaattatccgaggacggactattccgggtcgctcttggaatacgccgaagttgttacggcggagttctagagtaaagcctaagggctaagtccctctaccgaaccgacgatcctaaacgcgtttcggaaagttggcgaaactccaaacctctcaaagagccgtcgaagaggcaaaccaagtagcttaaggtcggccaataggaccttcgctagtccaagggaaaccggagattaaggaccttctaacccgctaccaacaacaacaaggtaccgaaacggaacccttccgtcaaacttgaacgcaaggttagggagggtccgattaacggcaacgattacttgcttgtaaggcggagcgtctagggactctatagttggacgagcctccgcaaacaagagctattattaccggaccgggtttggtcctcaatcgacgtacggaggtaaggaattgcccttggagcgggaaaccgttagtttacccgtacgggtttcgttccttgcctagcaaataaggtcgcggttatacgccgttgcttagaagccaacgaccaattgggtagggcgacgggacgatagtttgtccgttgttgctatcctctcgactatcccgttaacgggctaactataccgttgacggacgaaggatccgtaaagggccgttgttgactcaagcggcaatttccgaagcgcaagtaacgagaaggcgtcaacctacttgaaacccgaagaggagccgttgacaacccaaagaaggaattgccggcccaagcttcgttcgcttgttaacttgtagtccgacttggcgaccttgtatccgaataactcccgacgaaggtcgttcgatactcgagttcggccgggattagttcctctcaaggaaagggcaacgaactagccaaagtacgcaaggtaccctagaattggcaaggaacgggtttgctaccgacgcctaattggccgacaataggaggctaaacgttgttgccgtccgggtttattcgttccaaggctttggcctttattgcttcgctcgcgaacaatccgttctataacgcaacgcctattgggaaggcccggtttgtttcgccaatttgccaagtcggtcctcttgatcgaacgctaactatcggagacgttcgtccgccttaaggaacgcttgttaaggattccgcggaaggtatatccaagaaaggcccgacgtcttgcctaacccttatcctaccttccgacaattcctccttggcttgacgattacgagaagattcgagccgtccgaggtactaatctaaaccgggcgagtcgcaaagtttaacgccttaccctacccggaaatttcaaccaaccgaagcgcgtcgaaccttaaatctttccggcccttgctagtcggtacgttcctcctctcctttacctcttaccgcttaggctagtacgggacgttggaagtaaaggacgcgggcctacaaacgctattacccaagttggactccgggcaacttagaagggtcgttaaggcgctagagcggaagaaccaagttggatagcggacccaaccaagattattcgaggaaagcgcctagtccgttcctccaaggcgaaatttggaggaccgacttgattcggagcaagaagttacaagggccgacaagacaattcgctcaacctcgaacgtcctaaccgtctctcttgctactctaccaaccgatagggctccctaatatcgagaggtttgcctctcgtttcctcttaaccggaagggttacaaggcttgaaccgataacccaagagctaccggcgtaccgttccgaataaattaccgggatcggacgtccttcgaattgtccaactccggcctatttgttggcctccgtaaacgacgaattgcgtccggattgactttcccggagatcaagtccaagacggcaatacgttgggcctcaacttaggtcgccgagctatctttcccgactaacttgattggttgcgcgtcgatagtatccaattggactttcggcctagggacgttccgtataccaaggcttggacccaacttattgagcctaccgcttcgtaggctaaccttctaggccggcttaagacttaagaacgccgacgcgacaagtttacccttgcgctctctaaggttgcggtaagaggaattgagcctaaacccggcgctataacgacccaagtctaactacccaattccggcttgcgttgctcgcaatcctaactaattgagccgaggaaccaagattggccgacccttccggtttgaaaccaaggagctaacgttccaacttggcccaaagttgaaactcgggagggccgtagggaagtagtatctaccgtacctagatacactcaatttgtactcggggttccgttttacattccaggaaacgttgttgcgataggtattgaagcgagggtagaagggctattagttacgggcgaacgggatttggaccctcctttacttggcgctctagaatcaagggccgtaccttctttgtccttcggaaccttgcttgtcgtcaactcgctacttagatctcggcgacggaccaactataagtaagccctaaggcgtttgggtcgcaatataacggcttaggcctttcgccctttggacctacaagcaagagagagggatcaaacggttgggatttcggatccggacaattgtatccctaaccaacgacgacgttgttgcgcgttagggtcctttgaacgcgaagggaaaggattggcgaccttgggctaattactttcgcgcgtaacctacttcgaccttgagtacgccgaaattgctcggaccttctacgtatcgtaacaaccgctctacgacggccgtttcaaacaacgttggctaccgtagtccgtaggtacgagtaggaggaagggaggttagggtaattcttccggacgaaatcaagtagcgttgggccttagtcccgattcgaaggagctacttgtccgctacctccaacttagctccaacctaacccgggattggcaaataacgtaggccaaacgtcttgagttgacccaaacgtagttgggacggaacccttcggctttagaaggaattagcccgctacaaggccaaacaacgacgggcgtttacgtagttgttccgttgacgttcaaagggacttggtcgccaatctactcgcggaaacaacttgcggtcggtaagtaagcgacggtaagaaagtcctcttccctagttcaagcggcctaattgaccaacgaacgtacaattcgacgctagggagatatagcgcctagggaaagggcgggataattccgattccgtacggcaagttcgtcttgttcccgggcttcttagtttacggtcctacgcgttagagtcgttgcgagtcggttccttattcggtttccgccaagagggtctataggactagtacaacccggtcggcccttctttgcttaaaggctctcctccttggcgcgtaactttgtcaagcgttgcgaagtaggtcaagaggaaaggcaacgctagcgtaactccgtccaaacgttacttgtcggtttgacggagaaaccgtaacgacctccttgggtaagttcgatcttggctagcccttgctagtcgtctctcctcctttacgtaaggacaagcgtagcgctaactaggctacctctctccaaaccaaggaattgggtagcgaggtatccaacctctcaagtcggaataaggcccgttcccgtattatccaagacgtctaaccggactaccaacccgaactagggcggaattgacgtaatattccgtcccgccttaatcgactcgtacgggatacggttaagtcttgctcgggtaatcttggcggttccctaactttcttcgccgtagaagtacgggtcgaaggcgagggatttgggtattggaggttacgctagccgattgtcaaggccgcttatagattggcctacttaggctagattgcggaactcgaatcgacgtttcggtagccgaagaaagggaacgcgcctataagtacctcgaaacccttcgttgagtagcctctcgaggataagcggttattgcgtcgggaagagttctagttacccgtacgccgtatccctatctttaacgggtaagccttgaccgcggtagatagtagggatcgtactcaaagggaagcgctcctcgttagaaaccgacgaagcggcttgctatacaacgccgtactttaggcgatcccgtttcaaacccgcaaagcaagtctcgcttagcctaatctttggcttggccaacgtaacggctacttgggcgtctttactcgcctaaattgccggcctctctatctcaacgggagcttggaattgggtaacttgctaggcaaaccctagaggttcaacaaggtccgaccaacggagggtatcaagttgagtaggtaggcaaagaagggcgaacggttaacggaacaagagccggtaagcgtcttggtatcttcgttgcctcaacgtcggtctagttgactaagtaccgtcgaggcgtttgcccttgagaacggttagaacctctcgtcgagtagaggtttaaggtcggaaagaaatcggtccgcggtatactctactaccgaaaggtcgtcccggtagctagctaaacttggaaactcgggcccaatctccaagacaagtacaaggcctagcctcggcttaaaccaacctcggagttgcttaggctacttcgtcaaggtaggctaatcgggcgttgattaaacgctacggtaccgaaagtaccggacgtaacctactccgtttacgccctagtcgaaagggtaacgcttgggagttcgttgttgcgcccttacgtcaaagacggataaggagaagccctttcggtagttcgcctaagttgaactccgttgcctacgactaaccaaccaaggttgtccgtctcttaaggctcggaacttcaactcgggagacttgttcgaacgatattcgttgcgggttcggctttagaacgaaaccgaagggacggaacgtttaaaccctcctccttctttccgccaaagtcgaaggactcgctttaacgcccaaataaccgccaaggatcggcgcgtagtattagttcgtcttgccctatttgggaacttccctccgcaacctttacgctaccttacttggacgagagccaagtagagccaacggttacctcttagggcccggtttgtaatataaggcctcgctaaggaagagcgcgaaccgcttaaatctccttagcggcctcttcttattaccggcgactacgcggaagaggacaaattcccaactcgttcaattgggcccgactttggtccaacttagttggagtctagacctaggtattggcccggttaaccgggttagggttgtctagggagaatatcccgtgaagcttgagtggaatcgccgctgacgtgtgaggcgctagagcatagaccgaaaccaattggtttctagagcggtagggtagctagggttaacttacccgtcaacggccgttagcccgttaactacaaaccgttgggacctttgggcaacttgttgctacgactttaagctcgaacccttcgagagagcaagtacctagctcggaagtaacggcctagatttactcctcgccgaagtaggaagatcccggtcgacgcgttgtttctaggtttgggttgctccttgggacctttgagtaacgagttcgaaccggaccaatacgttgccgtacctctttaatagtcttgaccgcctcggttggcttagacggtcggttagcaatatccgtcctctagaagggtacccgaaacttgcgtagtaacgcctaaggacgtcctagacctaaccgaaagaccctaggagcaaggtacctaaggtcctatagtccgagccaaaggtcgtagttgacttagaggccgggatcaagagggaagggacggatcaaactacttgaaggccttcggccgaattgacctcaaatattcccgggaccgttgggaaccttgctttccgagtctccgctagtcgttgaacttgagggatcttctactccgggaaaccgtcgcaaaccgacaaggcaataggacttctaatccgtcctagggagccttcaagagtcgacgagtcttggtaattcttggacgtttacgcggtacgagcgttcgcttatcaaggcctccgatactaacttaaagggctcaaccggttgtcctaatccggagtactaagtacgggcaaactcggtaggacccgttgaaagaacaaagcgacccgaaagacttgcgttattccggctttcccttctcaagtcaaaccgtccgcttctttggaggttgttgaaggcctccctcaactcctaaccaaacaagaccaaggagcgaggagagaaattgcgaagcgaccctaaccgagaaacttgtacgggtcaatcgcccaaccaactattacccaaccgtcctcctacttggctacaagggttagcgtcaagatttggcgcttaggaaacctcttgctagagcaaagaagctcccggaagatccaaaggacggtagggttctagcttcaaatcgcgcttgacctaataagccgacggagccctcggtaaagtcaattcgggtagcttagaagccgcaaggagattacccttcgttagtccctccttcccgattcgagaggattacaacggaattgggaccgaagtaggccggaagttacggagttcgtcgtttcaaaccctcccaaacttacgggacgtattgtagggacccgggattccaaccctaattgttactcccggcgttgagttagagccttagacaaggtcgccaaacggaccctaactaagacgagttagcccgccaactaataccctagtaacccttcggtaaggtcgccgactccgatatcaaacgcgttagaaaggcgacggcaattggtacggtctacgttgtcttaggacggttcgggaacaacgaggaaagacaataggcgctaagccgttgagtcccaacgtaagagaggagcaagacttgaaggcttggtcgggtctaaggcaaaccctttagcgattgcccgaggaacggtagttgatccgtaggttggagtctccgattaatccgttccggtttctccctcggttcgtcgagataagggcgattagaggtttagcgccgtaagctaatccgaacgactacttccaattgctcggcggttacaagcttgctcgttacgacggcaagatagtcgagggtttacgtataccgggagctacgccttcttggtttccaaatccggtcgctaagggtacggtttaatccggtagccgacttggtaaccttattcgggaacgacaagcgcaagcgatcccaaacaaaccgtaactcgggtaatcaacaagttgccgtccgctttacctcgttgtaagtcgggttagggcccaatctaccaaccaagggttaacctcttctattggagacggccgataagctttgcggccctcctaaccaattgagtacgttgcgttcttcccgcccttatacgtacctttcccttcttgtacgccgaccgttgacgaagtacctctagcgctagctatccgaattgttacgggctataggtcgtaaccgggatcgaagctcctaattccaagaccctttggcaacgattctaccaacggacgaggagctacgttcttacaagaacgcgcgaggaaggttaggccttaaacaaggttcggtacgattcccgggattctagcggtttagctccaaggcccttagagggtcctttaacgtttaccaatccggacgtcggacaactattgcgattggacccaaccaactcttggcaattggaaactaagggccgacaataggcctacgggtagtaggagtaagacggtttcgttggcggtaactagacgcgagttgacaacccaaacaaggtagtctacgaagcggactcttcttccggagtcccgactagattacgaaggacaaagtcgcaagcctcaagccttgggttggagttctaaggagcccgtataaacgcaaaggaggaaacgacttgaagccgaggaaaggcggaagtaatacttgaccaaccgcaagtcgctctattgtcgctagctagaaggtcctaccgagggcgtttgaattagacgtttggcggtcaagcaaacgggacgggagaaacctcttgcgttctctagtacttccttgggagtcgaacgaacaagcttgtagaacctacggtagcggccttagggtattacgttgttgacgaagggcggtactcccttgttaacttgaaggcccggttggcttctaccaatcaagggtacgttgggtcttgacggctaggttaatcgcgctccaattggcgcgtcctaatatagctctattgggaggacggttacgtattcggcctaaggttgggttccttgaacaaggtatggcacgcctaatctggacacaggagaggtctttcatgcgtatagtcacatccgaaggccgggagaagcaattggagttgaccctatctccgcaagacctttgcgcaaccttgtaaggacgttgaaacggttgtacccaaacaacggccggttgatctaagggtaatccaagggcttacgggagcaacaagctagttcggactagagggtcctctagcgataaagctaacgttcgagaacgccgccgctaattgttgttgtcaagctaccgaggcttcgcaatacgatcgaagctagtccgacggctcaaaggaaagccgtaatcgtcgcccaactaccaatcgaaccttgaggtacccaagaggtcaataccctatcctaggcgaacgaaggatacaagggacccaatagcaaggttggatagtcgcccttggtattgggccaagaagaggcgatatcgtaaccgttgatccgttgagttacttcgaccgctctaggttcttacggtcggtactcgcctaaagcggcttgtttcgaccttaaaccgaacgggcaagacgaattgaggaacaaggctcccaaccaaggttgaacgttagcggcaattgatccaaccgtcgtaaccttgttgtagggtccgtttaaccgctaagcctaaggaagccgagtcctaaataaaccaagccgcggactaggcaacgggttagttgtccgataattaccgagcgaccggaccgcaaatacaagttagtagaggcggacgtctaacgttgctcgaattggatcgactcgcaagaccctagttgggacttcccttgggattacctcttcgccttctagcaaggttgttgcggacggtagttaggtcgacttggtcgttcttgaccttcttcccgtccctaagctagggtaaccaaaggttcgcgattcgtccgggtaaatttgcgtagcgcgttgcttctaataaccggatcctccggatacccaatcgttggttaactctcgtccgattaggcgtcggcgtttacgttaagggttagaagaggcgggcaaaggaacttaacaacgttacccggaaagcgaccaaaccgaagtcgatcctcttgacgagaggagaaggaatttccaaagagcgggcttcaaacggctctagcggcttcgtctaaaccttcgcaaccaacttcccaagacgacttggaacgcgtagagtaaggccgctttgttgttgggatcttcttccaatccggatatccggtcctaccgaagaaggtattggtaagggatcgcctccgttgaccgattacttacttgatcgcggccgttagcaagtccggtatacgcctcaatagtttacgcaagcggaggaagcttggttgggtaaccgaagggaagctccctacttagacgggtttggagtttacgtccaaacgctcgcctcttattaccggaagccctcgatctttggaccgcgtaggacaactacctacgtcgctcttaaattcgaccgacaacgttcgtaaggcctacggcttccttggttgttacggcgttcaatagcaagcgttagcgaaggccgggagtaataataggagggagtcaagagagattatcggtcggagccaagttgagtcggacccaagttaaaccgtcgtcgtacggttaatcctctaaccggtaaacctccgttcgttgggtcgatccaaacctttctctcctaccgaccgcttaagcctagtcgaggtaccaagctaagtagtcaacggacgtaacttcccgtcgttacgcttggactacttcttaagcggcgacaaggattggacgtccaagtacccaatctcgagtagctcaattaccgggccaactacccaagactaaggcgggttgttcgagctaaccaaggactcccgacgtaattgtacccgccgatcgtaaattcgcccaataagcggattgggtttgtcggactccgacgttaacgaacccaataagggatagttaagggcccgacgaagttaaagccggacggctaaggtacaattgttacggcccggttcctttgtccgtttctacctcaaccttgccgcgaacgactcgtttaaccgaaggctagaaactttggcgaaacggtacttacgagggacctagctttgtaagcggttatccgcgcggacttatttggaggtcgttgggcctaacctcttggttgtagacccgataccgtcggcttcaattggaagatcgaaggctccttagtatcgggaagggtagttcctacgcgttaggaaaggctcttaaacgcccttgagcaagccgtcaacgtttagtcgttgttggacccgaggttgtaggtaagtaggatacgactcgggtccgcgaagaaatataggtcggcaacttagagtcccgagctttagggaacaacaaacctcggctattcctacgcggaaacgctacctaaagaagggcaacgacaagcgcgaacggtataacgggtacgactactcccgacttaggttaccttaacgggcaaccgaacgcttagctcaaacgttcggtcccaaccttcaattgtcgggtccctctagggttacaatttgctccgcctctagggaggtttcgctatagcaagttcgttggtaggcttgacgggtttggttgcgaggcaatataggaggtattgggtctttaggacgagcgagtttggccgcaatcgaatttgacgcctagggcgaaccggtttaaccgaagcaactattggttgggttcaagggttgggtctaccggaaatcgagttgggtcgtactccggagttctcaagccaacttaggcaactcaaacgattcgtccgctaactccgacttggttgtcgtataagcgctctcctttggagtctttaacgtccgggtttacgagactacgcgattgcctcctaaagcctttaaccggtagccgggcttaagcccgaaagagctttgaaacgcggatatccgcttgttgtcaattgtcgaccggccttagctaagggacggagggctagtttatacgggtcggagattcaatatgtgtcgtctatcctcagtcggtaactgcgcatagttggctctataggttcccggaacttcaatcgttccgctcctttaggttgaaggcgtttacggcgggtaaccgtttagggttagcaaggcaacctcgaggaaataggtcgggtattgagggaagtactagaagcttcccggagggttagaaggagggtaacggttaacgggacgaggaccaatttgtcggttgtcaacggttagttcgggtcaaaggaaaggacggatactttgctcggccgacctagaagttgagaagagaggtccggaggttgggtttcaattaggttgccgtttagggcctagggtaacaaggtttggtttcgagccgcttgactacggatttgaccaaaccaagcgtttaaggtcacatcgcatgaat

(19) United States
(12) Patent Application Publication
(10) Pub. No.: US 2022/0351807 A1
(43) Pub. Date: Nov. 3, 2022

(54) BIOCOMPATIBLE NUCLEIC ACIDS FOR DIGITAL DATA STORAGE

(71) Applicants: CENTRE NATIONAL DE LA RECHERCHE SCIENTIFIQUE, Paris (FR); SORBONNE UNIVERSITE, Paris (FR)

(72) Inventors: Stéphane LEMAIRE, L'Haÿ-les-Roses (FR); Pierre CROZET, Orly (FR); Zhou XU, Arcueil (FR); Alexandre MAES, Bagnolet (FR); Jeanne LE PEILLET, Paris (FR)

(73) Assignees: CENTRE NATIONAL DE LA RECHERCHE SCIENTIFIQUE, Paris (FR); SORBONNE UNIVERSITE, Paris (FR)

(21) Appl. No.: 17/766,006

(22) PCT Filed: Oct. 1, 2020
(86) PCT No.: PCT/EP2020/077497

§ 371 Date: Apr. 1, 2022

(30) Foreign Application Priority Data
Oct. 1, 2019 (EP) 19306247.8

(51) Int. Cl. G16B 50/30; C12N 15/10; G16B 50/50; G16B 30/00; C12N 15/66; G06N 3/12

(52) U.S. Cl. CPC G16B 50/30; C12N 15/1065; G16B 50/50; G16B 30/00; C12N 15/66; G06N 3/123; C12N 2800/10

(57) ABSTRACT

A device for the storage and/or the editing of digital data including at least one double stranded, replicative, composite nucleic acid molecule. The composite nucleic acid molecule includes both digital data-encoding and non-digital data-encoding nucleic acids. The non-digital data-encoding nucleic acids may allow indexing and/or the provision of metadata for the flanking digital data-encoding nucleic acid. The composite nucleic acid molecules may be pooled to constitute an array and arrays may constitute a DNA drive, which represents the physical support on which the digital data are stored.

### FIELD OF INVENTION

The present invention relates to the storage of digital data onto a biomolecule. More particularly, digital data may be stored onto a double stranded, replicative, composite nucleic acid molecule and further be easily retrieved upon sequencing.

### BACKGROUND OF INVENTION

Storing and archiving digital data are major issues in our modern societies. The current digital media stored in data centers are fragile, bulky and energy-consuming. Although optical media, magnetic tapes, hard drives or flash memory have been developed, their durability does not exceed twenty years on average. These data must be regularly copied onto new reliable media and this operation, which must be performed at controlled temperature and humidity, induces a colossal energy cost and requires huge amounts of raw materials. The amount of energy consumed by data centers reaches such thresholds that if the Internet was comparable to a country, it would be the 6^{th} largest consumer of electricity in the world, with an annual consumption of 150 TWh, which corresponds to 4% of the worldwide global energy consumption and represents approximately 40% more than the annual consumption of the United Kingdom. The carbon footprint of the data centers approximately corresponds to that of global civil aviation. Despite their energy cost, their carbon footprint and their increasing need for bulky area, data centers can only store 30% of the data we produce while our data production grows exponentially: “If today we are capable of storing about 30% of the information we generate, in only 10 or 12 years we will be able to store about 3%” (Dr. Karin Strauss, Microsoft Research). Given these general considerations, the data revolution, the big data market and the development of artificial intelligence cannot be pursued without finding innovative solutions to the problem of data storage.

WO2019079802 disclosed a method of decoding a nucleotide sequence, the nucleotide sequence encoding a value corresponding to a format of information, which includes converting a format of information into a sequence of binary ASCII bits, converting the sequence of binary ASCII bits into a sequence of ternary ASCII bits, and converting the sequence of ternary ASCII bits into a corresponding oligonucleotide sequence.

Taejin Ahn et al. (Genomics and Informatics, 2018, Vol. 16(4):e30) disclosed the storing of digital information in long-read DNA (approximately 1,000 bp), in which each bit 0 or 1 is encoded by a 16 bp nucleic acid unit, made up with a 4 bp signal sequence (TATT for bit 0 and ACCC for bit 1), flanked at each extremity by a 6 bp noise sequence (random sequence).

Existing methods for storing information in the form of nucleotide sequences (e.g., DNA or RNA molecules) have limitations and technical problems, among them: (1) they are usually based on short sequences of single-stranded oligonucleotides (<200 nucleotides), limiting the density and the quantity of stored information; (2) they usually require chemical or enzymatic synthesis in vitro; (3) they are usually based on an index organization system which is constrained by the physical medium, namely short nucleotide sequences, and is therefore of limited effectiveness; (4) they are usually not compatible with manipulation using a living organism.

Digital data storage in cellular DNA has been discussed, e.g., by Dagher et al. (Evolutionary Intelligence, 2019). The authors provided suggestions to conceive a nucleic acid molecule suitable for being stored in a cell, and explicitly recommended protein-coding DNA (pcDNA) as a preferred environment for DNA encoding due to its ease of implementation, and because pcDNA is well understood via the codon and dominates the genomes of virus, prokaryotes and yeast.

There is still a need for providing the state of the art with means for storing digital data that can sustain encoding of large amounts of data, and can further be biocompatible, i.e. that can be copied, edited, written and/or read using living organisms.

### SUMMARY

One aspect of the invention relates to a device for the storage and/or the editing of digital data comprising at least one double stranded, replicative, composite nucleic acid molecule comprising a nucleic acid of formula (I):

5′-([UP]-[DB]-[DO])ₓ-3′ (I),

wherein,
[DB] represents a digital data-encoding nucleic acid having a length of from about 8 nucleotides to about 10⁶ nucleotides, preferably from about 500 nucleotides to about 5,000 nucleotides,
[UP] and [DO] represent a pair of non-digital data-encoding nucleic acids, each having a length of from about 0 nucleotide to about 104 nucleotides, preferably from about 10 nucleotides to about 200 nucleotides;
x represents 1 to about 105.

In some embodiments, the composite nucleic acid molecule has a length of from about 500 nucleotides to about 10¹¹ nucleotides, preferably from about 103 nucleotides to about 105 nucleotides. In certain embodiments, the nucleic acid of formula (I) has a C+G percentage of from about 35% to about 65%. In some embodiments, the nucleic acid of formula (I) does not encode one or more RNA(s), preferably does not encode one or more mRNA(s). In certain embodiments, the nucleic acid of formula (I) does not comprise one or more initiation codon(s) and/or comprises one or more stop codon(s) per about 200 nucleotides in all 6 reading frames. In some embodiments, the nucleic acid of formula (I) does not comprise one or more restriction site(s) for the enzymes or isoschizomers thereof selected in the group consisting of BamHI, BsaI, BbsI, EcoRI, FokI and I-SceI. In certain embodiments, the nucleic acid of formula (I) does not comprise one or more repeat(s) of at least 4 identical nucleotides. In some embodiments, each nucleotide of the [DB] nucleic acid encodes 1 or 2 bits of the digital data. In certain embodiments, the [UP] and [DO] nucleic acids each contain at least one barcode-encoding nucleic acid and/or at least one metadata-encoding nucleic acid.

In one aspect, a method for storing digital data comprises the steps of:
a) assigning to said digital data at least one double stranded digital data-encoding [DB] nucleic acid sequence (S_{DB}) and at least one pair of non-digital-data-encoding [UP] and [DO] nucleic acid sequences (S_{UP}) and (S_{DO});
b) synthesizing the at least one nucleic acid of formula (Ia):

5′-([UP]-[DB]-[DO])-3′ (Ia),

from the sequences (S_{UP}), (S_{DB}) and (S_{DO}), respectively;
c) assembling the one or more nucleic acid(s) of formula (Ia) so as to obtain a double stranded, replicative, composite nucleic acid molecule comprising a nucleic acid of formula (I):

5′-([UP]-[DB]-[DO])ₓ-3′ (I),

wherein x represents 1 to about 105;
d) storing at least one pool comprising from 1 to about 10⁹ composite nucleic acid molecule(s) of distinct sequence and comprising a nucleic acid of formula (I) obtained at step c) into a storage cell.

In certain embodiments, the method further comprises the step of:
e) organizing and grouping the pools obtained at step d) into at least one array comprising from 1 pool to about 10⁶ pools, preferably about 96 or about 384 pools.

In some embodiments, the composite nucleic acid molecule obtained at step c) is a plasmid, a cosmid, a prokaryotic chromosome or a eukaryotic chromosome.

In certain embodiments, the method further comprises the steps of:
c1) amplifying in vivo the at least one composite nucleic acid molecule comprising a nucleic acid of formula (I) obtained at step c); and
c2) extracting and purifying the amplified composite nucleic acid molecule obtained at step c1).

In some embodiments, step c1) is performed in vivo by a living organism, preferably a microorganism.

Another aspect of the invention relates to a method for retrieving a digital data stored by a device according to the invention and/or stored by a method according to the invention, said method comprising the steps of:
a) sequencing at least one nucleic acid of formula (Ia) comprised in a double stranded, replicative, composite nucleic acid molecule comprising a nucleic acid of formula (I), so as to obtain at least one nucleic acid sequence (S_{UP}-S_{DB}-S_{DO});
b) converting the at least one nucleic acid sequence (S_{DB}) into digital data; wherein step a) is optionally preceded by step a0) of amplifying the at least one nucleic acid of formula (Ia).

### Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Digital data” refers to data that can be managed by computerized machines. As used herein, the expression “digital data” is meant to refer to data represented by a binary system. As used herein, a “binary system” refers to a language composed of bits “0” and “1”. Non-limitative examples of digital data may be program files, text files, music files, image files, video files and combinations thereof.
“Storage” or “storing” refers to the action of keeping an item in a specific place for future use or for safekeeping. More specifically, the expression “storage of digital data” is intended to mean the action of safely keeping the digital information for further use.
“Editing” refers to the action of assembling an item by cutting, pasting and/or rearranging fragments of said item. As used herein, “editing a nucleic acid molecule” is intended to refer to the modification of said nucleic acid molecule by inserting, deleting or replacing one or more nucleotide(s) within the nucleic acid's sequence.
“Biocompatible” refers to the ability to be handled by a living organism. As used herein, a “biocompatible nucleic acid molecule” is intended to refer to a nucleic acid molecule that is compatible with replication and manipulation in/by a living organism, such as e.g. copying or editing.
“Replicative” refers to the ability to be replicated in vivo by a polymerase, such as, e.g., a DNA polymerase, i.e. to be exactly duplicated, within the margin of error of replication mechanisms of living organisms. As used herein, a “replicative nucleic acid molecule” is intended to refer to a nucleic acid molecule that can be copied at least once. In some embodiments, the nucleic acid molecule according to the invention is selected in the group consisting of a plasmid, a cosmid and a chromosome. In practice, a replicative nucleic acid molecule comprises one or more origin(s) of replication (also termed ORI), including one or more centromere(s) (for chromosomes).
“Composite” refers to an item made up of distinct parts or elements, which are combined together. As used herein, a “composite nucleic acid molecule” refers to a nucleic acid molecule that originates from fragments of nucleic acids that may specifically be designed in silico, synthesized and assembled and/or created in vitro or in vivo.
“Barcode” refers to a patterned item that contains information about the object it labels, in order to uniquely identify said object from a collection of distinct objects. As used herein, a “barcode-encoding nucleic acid” is intended to refer to a non-digital data-encoding nucleic acid that allows the labelling and/or the indexing of the flanking digital data-encoding nucleic acid.
“Metadata” is meant to relate to basic information about the digital data they are referring to, such as author of the digital data, date of creation of the digital data, date of modification of the digital data, data content and file size.
“Nucleotide” and “nucleic base” are meant as substitutes for one another and are intended to refer to the nucleic building block of a DNA or RNA molecule. As used herein, a nucleotide refers to a purine Adenine (A) or Guanine (G); or to a pyrimidine Cytosine (C), Thymine (T) or Uracile (U). For DNA nucleic acids, A refers to the dAMP deoxyribonucleotide; G refers to the dGMP deoxyribonucleotide; C refers to the dCMP deoxyribonucleotide; and T refers to the dTMP deoxyribonucleotide. For RNA nucleic acids, A refers to the AMP ribonucleotide; G refers to the GMP ribonucleotide; C refers to the CMP ribonucleotide; and U refers to the UMP ribonucleotide.
“Array” refers to a solid support containing a collection or a set of nucleic acid molecules, preferably organized in one or more pool(s).
“Amplifying” refers to the action of multiplying a compound of interest. As used herein, the expression “amplifying a nucleic acid molecule” is intended to refer to the multiplication of the number of copies of said nucleic acid molecule, taken as a template. Unless otherwise specified, the terms “amplified”, “duplicated” and “multiplied” are intended to be used as synonyms and may therefore substitute one another.
“Extracting” refers to the action of withdrawing a compound of interest by physical and/or chemical process. As used herein, “extracting an amplified nucleic acid molecule” is intended to refer to the removal of the nucleic acid molecule from the living organism that has amplified said nucleic acid molecule.
“Purifying” refers to the action of obtaining a pure, or substantially pure, compound of interest, from a mixture of compounds. As used herein, the expression “purifying a nucleic acid molecule” is intended to refer to the removal of the impurities from a mixture comprising said nucleic acid molecule, so as to obtain a pure, or substantially pure, composition of said nucleic acid molecule.

### DETAILED DESCRIPTION

The inventors have shown that digital data, also referred to as computerized files, may be easily stored onto double stranded, replicative, composite nucleic acid molecules. The inventors have engineered nucleic acid molecules (in the form of DNA molecules) comprising both digital data-encoding nucleic acids and non-digital data-encoding nucleic acids. The said non-digital data-encoding nucleic acids are advantageously used for assembling, replicating in living organisms, indexing the digital data and/or providing metadata. The replicative properties of the composite nucleic acid molecules according to the invention allow their easy handling, in particular their amplification and/or their editing in/by a living organism.

This invention relates to a device for the storage and/or the editing of digital data comprising at least one double stranded, replicative, composite nucleic acid molecule comprising a nucleic acid of formula (I):

5′-([UP]-[DB]-[DO])ₓ-3′ (I),
wherein,
   [DB] represents a digital data-encoding nucleic acid having a length of from about 8 nucleotides to about 10⁶ nucleotides, preferably from about 500 nucleotides to about 5,000 nucleotides;
   [UP] and [DO] represent a pair of non-digital data-encoding nucleic acids, each having a length of from about 0 nucleotide to about 10⁴ nucleotides, preferably from about 10 nucleotides to about 200 nucleotides;
   x represents 1 to about 10⁵.

It is understood that the composite nucleic acid molecules according to the invention are biocompatible, in the sense that they may be duplicated and edited in/within/by a living organism.

It is understood that the composite nucleic acid molecule comprising a nucleic acid of formula (I) comprises x nucleic acid(s) of formula (Ia):

5′-([UP]-[DB]-[DO])-3′ (Ia).

In certain embodiments, the digital data consist of binary digital data. In practice, the binary digital data are represented by a succession of bits, wherein each bit is represented by either bit “0” or bit “1”.

In some embodiments, the digital data may be selected in a group comprising program files, text files, table files, music files, image files, video files and combinations thereof.

In certain embodiments, a text file may be under a .htm, .html, .rtf, .txt, .ccp, .py or .xml format. In some embodiments, a video file may be under an .avi, .mov, .mpeg or .mpg format. In certain embodiments, an image file may be under a .gif, .jpe, .jpeg, .jpg or png format. In some embodiments, an audio file may be under a .mp3 or .ogg format. In certain embodiments, the file may be under a .exe, .doc, .pdf, .ppt, .ps, .xls or .zip format.

It is understood that a nucleic acid molecule according to the invention is a double stranded nucleic acid molecule, i.e. comprising two antiparallel complementary nucleic acid strands. In practice, one strand is oriented from 5′ to 3′ and the complementary strand is oriented from 3′ to 5′.

As used herein, the “replicative” property of the nucleic acid molecule according to the invention refers to its ability to be duplicated one or more time(s) in vivo in a living organism, in particular by a polymerase, more particularly by a DNA polymerase.

In practice, the assessment of the replicative property of a nucleic acid molecule may be performed according to any standard method from the state of the art, or a method derived therefrom. Illustratively, the replicative property may be assessed by the increase of the number of copies of said nucleic acid molecules in/by a living organism and/or the ability of the living organism to transfer the nucleic acid to its progeny.

In some embodiments, the living organism is a microorganism, in particular a bacterium, a microalga, an archaeon, a fungus, a phage, a virus or a yeast. In some embodiments, the living organism is a prokaryote. Non-limitative examples of prokaryotes according to the invention include bacteria, such as actinobacteria, chlamydiales, cyanobacteria, firmicutes, proteobacteria, spirochetes, thermotogales; and archaea, such as euarchaeota, crenarchaeota. In certain embodiments, the living organism is a eukaryote. Non-limitative examples of eukaryotes according to the invention include protozoa, algae, plants, fungi, animals and their respective cells thereof.

In order to be replicated, the composite nucleic acid molecule according to the invention possesses at least one origin of replication, namely one or more sequence(s) of nucleotides recognized by a replication initiation machinery. Illustratively, archaeon and bacterial origins of replication include oriC. In practice, most bacteria may have a unique origin of replication; an archaeon may have one or more origin(s) of replication; a eukaryote may have multiple origins of replication, in particular in the form of centromeres. Within the scope of the instant invention, the term “multiple origins of replication” refers to at least 2, 3, 4, 5, 10, 15, 20, 25, 50, 75, 100, 150, 200 origins of replication per nucleic acid molecule.

In certain embodiments, the composite nucleic acid molecule has a length of from about 500 nucleotides to about 10¹¹ nucleotides, preferably from about 10³ nucleotides to about 10⁵ nucleotides.

Within the scope of the instant invention, the expression “from about 500 nucleotides to about 10¹¹ nucleotides” encompasses 500, 600, 700, 800, 900, 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰ and 10¹¹ nucleotides.

Within the scope of the instant invention, the expression “from about 10³ nucleotides to about 10⁵ nucleotides” encompasses 10³, 2.5×103, 5×10³, 7.5×10³, 10⁴, 2.5×10⁴, 5×10⁴, 7.5×10⁴ and 10⁵ nucleotides.

It is understood that the nucleic acid molecules according to the invention are represented by a sequence of consecutive nucleotides.

In some embodiments, the nucleotides of the composite nucleic acid molecules according to the instant invention are represented by nucleotides selected from the group of deoxyribonucleotides, ribonucleotides, and analogs thereof, more preferably deoxyribonucleotides. As used herein, a deoxyribonucleotide encompasses dATP, dCTP, dGTP, dTTP, dADP, dCDP, dGDP, dTDP, dAMP, dCMP, dGMP and dTMP. As used herein, a ribonucleotide encompasses ATP, CTP, GTP, UTP, ADP, CDP, GDP, UDP, AMP, CMP, GMP and UMP.

In certain embodiments, analogs of nucleotides may be selected in the non-limitative group comprising 2-Amino-ATP, 8-Aza-ATP, 2′-Fluoro-dATP, 2′-Fluoro-dCTP, 2′-Fluoro-dGTP, 2′-Fluoro-dUTP, 5-Iodo-CTP, 5-Iodo-UTP, N6-Methyl-ATP, 5-Methyl-CTP, 2′-O-Methyl-ATP, 2′-O-Methyl-CTP, 2′-O-Methyl-GTP, 2′-O-Methyl-UTP, Pseudo-UTP, ITP, 2′-O-Methyl-ITP, Puromycin-TP, Xanthosine-TP, 5-Methyl-UTP, 4-Thio-UTP, 2′-Amino-dCTP, 2′-Amino-dUTP, 2′-Azido-dCTP, 2′-Azido-dUTP, 06-Methyl-GTP, 2-Thio-UTP, Ara-CTP, Ara-UTP, 5,6-Dihydro-UTP, 2-Thio-CTP, 6-Aza-CTP, 6-Aza-UTP, N1-Methyl-GTP, 2′-O-Methyl-2-Amino-ATP, 2′-O-Methylpseudo-UTP, N1-Methyl-ATP, 2′-O-Methyl-5-methyl-UTP, 7-Deaza-GTP, 2′-Azido-dATP, 2′-Amino-dATP, Ara-ATP, 8-Azido-ATP, 5-Bromo-CTP, 5-Bromo-UTP, 2′-Fluoro-dTTP, 3′-O-Methyl-ATP, 3′-O-Methyl-CTP, 3′-O-Methyl-GTP, 3′-O-Methyl-UTP, 7-Deaza-ATP, 5-AA-UTP, 2′-Azido-dGTP, 2′-Amino-dGTP, 5-AA-CTP, 8-Oxo-GTP, Pseudoiso-CTP, N4-Methyl-CTP, N1-Methylpseudo-UTP, 5,6-Dihydro-5-Methyl-UTP, N6-Methyl-Amino-ATP, 5-Carboxy-CTP, 5-Formyl-CTP, 5-Hydroxymethyl-UTP, 5-Hydroxymethyl-CTP, Thieno-GTP, 5-Hydroxy-CTP, 5-Formyl-UTP, Thieno-UTP, 2-Amino-dATP, 5-Bromo-dCTP, 5-Bromo-dUTP, 7-Deaza-dATP, 7-Deaza-dGTP, dITP, 5-Propynyl-dCTP, 5-Propynyl-dUTP, 2′-dUTP, 5-Fluoro-dUTP, 5-Iodo-dCTP, 5-Iodo-dUTP, N6-Methyl-dATP, 5-Methyl-dCTP, 06-Methyl-dGTP, N2-Methyl-dGTP, 8-Oxo-dATP, 8-Oxo-dGTP, 2-Thio-dTTP, 2′-dPTP, 5-Hydroxy-dCTP, 4-Thio-dTTP, 2-Thio-dCTP, 6-Aza-dUTP, 6-Thio-dGTP, 8-Chloro-dATP, 5-AA-dCTP, 5-AA-dUTP, N4-Methyl-dCTP, 2′-deoxyzebularine-TP, 5-Hydroxymethyl-dUTP, 5-Hydroxymethyl-dCTP, 5-Propargylamino-dCTP, 5-Propargylamino-dUTP, 5-Carboxy-dCTP, 5-Formyl-dCTP, 5-Indolyl-AA-dUTP, 5-Carboxy-dUTP, 5-Formyl-dUTP, 3′-dATP, 3′-dGTP, 3′-dCTP, 5-Methyl-3′-dUTP, 3′-dUTP, ddATP, ddGTP, ddUTP, ddTTP, ddCTP, 3′-Azido-ddATP, 3′-Azido-ddGTP, 3′-Azido-ddTTP, 3′-Amino-ddATP, 3′-Amino-ddCTP, 3′-Amino-ddGTP, 3′-Amino-ddTTP, 3′-Azido-ddCTP, 3′-Azido-ddUTP, 5-Bromo-ddUTP, ddITP, (1-Thio)-dATP, (1-Thio)-dCTP, (1-Thio)-dGTP, (1-Thio)-dTTP, (1-Thio)-ATP, (1-Thio)-CTP, (1-Thio)-GTP, (1-Thio)-UTP, (1-Thio)-ddATP, (1-Thio)-ddCTP, (1-Thio)-ddGTP, (1-Thio)-ddTTP, (1-Thio)-3′-Azido-ddTTP, (1-Thio)-ddUTP, (1-Borano)-dATP, (1-Borano)-dCTP, (1-Borano)-dGTP, (1-Borano)-dTTP, Ganciclovir-TP and Cidofovir-DP.

In some embodiments, the nucleic acid of formula (I) has a C+G percentage of from about 35% to about 65%.

Within the scope of the instant invention, the expression “from about 35% to about 65%” encompasses 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64% and 65%.

It is understood that the composite nucleic acid molecules according to the invention may be safe for a living organism that would contain them and further safe to handle by the consumer individual. Therefore, the nucleic acids of formula (I) according to the invention may not encode a product that would be predictably harmful, in particular to the consumer individual, but also to animals, plants and the environment. As used herein, the expression “not harmful” is intended to mean that the product does not promote a disease or a disorder to the consumer individual, to an animal or a plant, and does not further constitute a pollutant for the environment. Illustratively, and non-limitatively, the nucleic acid molecules according to the invention may not encode a toxin, a pollutant, an enzyme, a poison, an antibiotic, etc.

In certain embodiments, the nucleic acid of formula (I) does not predictably encode one or more RNA(s), preferably does not encode one or more mRNA(s).

In some embodiments, the nucleic acid of formula (I) does not encode one or more RNA(s), preferably does not encode one or more mRNA(s).

Within the scope of the invention, “RNA” is meant to non-limitatively refer to antisense RNA, guide RNA (gRNA), messenger RNA (mRNA), micro RNA (miRNA), ribosomal RNA (rRNA), small hairpin RNA (shRNA), small interfering RNA (siRNA), small nuclear RNA (snRNA), small nucleolar RNA (snoRNA) and transfer RNA (tRNA).

In practice, the assessment of prediction that a nucleic acid of formula (I) does not encode one or more RNA(s) may be performed in silico, by analyzing the sequence of the nucleic acid molecule, e.g., for the presence of signature sequences for the initiation of transcription, such as promoter sequences.

In some embodiments, the nucleic acid molecule of formula (I) does not comprise one or more initiation codon(s) and/or comprises one or more stop codon per about 200 nucleotides in all 6 reading frames.

As used herein, an “initiation codon” may refer to the ATG, AUG, GTG, GUG, CTG or CUG codon.

In certain embodiments, the [DB] digital data-encoding nucleic acid does not comprise one or more initiation codon(s) and the [UP] and/or the [DO] non-digital data-encoding nucleic acids may comprise one or more initiation codon, with the proviso that the [DB] digital data-encoding nucleic acid comprises one or more stop codon per about 200 nucleotides in all 6 reading frames.

As used herein, a “stop codon” may refer to the UAA, UAG, UGA, TAA, TAG or TGA codon.

Within the scope of the invention, the expression “one or more stop codon per 200 nucleotides” encompasses 1, 2, 3, 4, 5, 6, 7, 8, 9, 10, 12, 14, 16, 18, 20, 25, 30, 35, 40, 45, 50 stop codon(s) per 200 nucleotides.

In some embodiments, the nucleic acid of formula (I) does not comprise one or more specific restriction site(s). As used herein, “specific restriction site” refers to a restriction site of determined sequence.

In certain embodiments, the nucleic acid of formula (I) does not comprise one or more restriction site(s) for the enzymes or isoschizomers thereof selected in the group consisting of BamHI, BsaI, BbsI, EcoRI, FokI and I-SceI.

As used herein, the expression “restriction site” refers to a nucleotide sequence targeted by a restriction enzyme, i.e. a polypeptide that has the capacity of cutting the said sequence within a nucleic acid molecule. In some embodiments, the nucleic acid of formula (I) does not comprise any restriction site from the following list: BamHI, BsaI, BbsI, EcoRI, FokI and I-SceI.

In some embodiments, the presence or the absence of one or more restriction site(s) may depend on the living organism hosting the composite nucleic acid molecule according to the invention. In practice, a composite nucleic acid molecule according to the invention comprising bacterial restriction site(s) may not be hosted by a bacterial living organism. Illustratively, a composite nucleic acid molecule according to the invention comprising restriction site(s) recognized by enzymes from one species may not be hosted by a living organism from said species.

It is understood that the nucleic acid of formula (I) according to the invention is advantageously synthesized and sequenced with high fidelity. It is known that repeats of at least 4 identical nucleotides may interfere with the high-fidelity synthesis and/or sequencing of nucleic acid molecules, as being prone to synthesis or sequencing errors.

In certain embodiments, the nucleic acid of formula (I) does not comprise one or more repeat(s) of at least 4 identical nucleotides.

Within the scope of the instant invention, the expression “at least 4 identical nucleotides” encompasses 4, 5, 6, 7, 8, 9, 10, 12, 14, 16, 18, 20, 25, 30, 40, 50 identical nucleotides.

As used herein, at least 4 identical nucleotides refers to series of nucleotides having the same nature, e.g. “AAAA”, “CCCC”, “GGGG”, “TTTT” or “UUUU”.

It is understood that the double stranded, replicative, composite nucleic acid molecule according to the invention comprises both a digital data-encoding nucleic acid and a non-digital data-encoding nucleic acid.

In practice, the digital data-encoding nucleic acid is referred to as [DB] for “data block”, and is intended to refer to a nucleic acid containing solely digital information.

Within the scope of the instant invention, the expression “from about 8 nucleotides to about 10⁶ nucleotides” encompasses 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 10³, 5×10³, 10⁴, 5×10⁴, 10⁵, 5×10⁵ and 10⁶ nucleotides.

Within the scope of the instant invention, the expression “from about 500 nucleotides to about 5,000 nucleotides” encompasses 500, 550, 600, 650, 700, 750, 800, 850, 900, 950, 1,000, 1,250, 1,500, 1,750, 2,000, 2,250, 2,500, 2,750, 3,000, 3,250, 3,500, 3,750, 4,000, 4,250, 4,500, 4,750 and 5,000 nucleotides.

In certain embodiments, each nucleotide of the [DB] nucleic acid encodes 1 or 2 bits of the digital data.

In one embodiment, each nucleotide of the [DB] nucleic acid encodes 1 bit of the digital data. Illustratively, Table 1 below provides the possible combinations.

TABLE 1
combinations for 1 bit/nucleotide

| | 1 bit/nucleotide | |
|---|---|---|
| Combination | 0 | 1 |
| #1 | A | C or G or T/U |
| #2 | A or C | G or T/U |
| #3 | A or G | C or T/U |
| #4 | A or T/U | C or G |
| #5 | A or C or G | T/U |
| #6 | A or C or T/U | G |
| #7 | A or G or T/U | C |
| #8 | C or G or T/U | A |
| #9 | G or T/U | A or C |
| #10 | C or T/U | A or G |
| #11 | C or G | A or T/U |
| #12 | T/U | A or C or G |
| #13 | G | A or C or T/U |
| #14 | C | A or G or T/U |

In one embodiment, each nucleotide of the [DB] nucleic acid encodes 2 bits of the digital data. Illustratively, Table 2 below provides the possible combinations.

TABLE 2
combinations for 2 bits/nucleotide

| | 2 bits/nucleotide | | | |
|---|---|---|---|---|
| Combination | 00 | 01 | 10 | 11 |
| #15 | A | C | G | T/U |
| #16 | A | C | T/U | G |
| #17 | A | G | C | T/U |
| #18 | A | G | T/U | C |
| #19 | A | T/U | C | G |
| #20 | A | T/U | G | C |
| #21 | C | A | G | T/U |
| #22 | C | A | T/U | G |
| #23 | C | G | A | T/U |
| #24 | C | G | T/U | A |
| #25 | C | T/U | A | G |
| #26 | C | T/U | G | A |
| #27 | G | A | C | T/U |
| #28 | G | A | T/U | C |
| #29 | G | C | A | T/U |
| #30 | G | C | T/U | A |
| #31 | G | T/U | A | C |
| #32 | G | T/U | C | A |
| #33 | T/U | A | C | G |
| #34 | T/U | A | G | C |
| #35 | T/U | C | A | G |
| #36 | T/U | C | G | A |
| #37 | T/U | G | A | C |
| #38 | T/U | G | C | A |

It is understood that the double stranded, replicative, composite nucleic acid molecule according to the invention may comprise, in addition to one or more digital data-encoding nucleic acid(s), one or more non-digital data-encoding nucleic acid(s).

As used herein, the expression “non-digital data-encoding nucleic acid” refers to a nucleic acid that does not contain any digital data information, but may contain information about a barcoding, an indexing, metadata, a security system, a proof-reading system, flanking the digital data-encoding [DB] nucleic acid.

In certain embodiments, [UP] and [DO] represent a pair of non-digital data-encoding nucleic acids having each a length of from about 0 nucleotide to about 10⁴ nucleotides.

Within the scope of the instant invention, the expression “from about 0 nucleotide to about 10⁴ nucleotides” encompasses 0, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 10³, 2.5×10³, 5×10³, 7.5×10³ and 10⁴ nucleotides.

In certain embodiments, [UP] and [DO] represent a pair of non-digital data-encoding nucleic acids having each a length of from about 10 nucleotides to about 200 nucleotides.

Within the scope of the instant invention, the expression “from about 10 nucleotides to 200 nucleotides” encompasses, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 and 200 nucleotides.

In some embodiments, the [UP] and [DO] nucleic acids each contain at least one barcode-encoding nucleic acid and/or metadata-encoding nucleic acid.

As used herein, a “barcode-encoding nucleic acid” is intended to refer to a nucleic acid that allows the labelling of the flanking digital data-encoding [DB] nucleic acid. In practice, the labelling properties of a barcode-encoding nucleic acid facilitate the data retrieval process.

In practice, barcodes may be obtained from an available library or generated in silico.

In some embodiments, the composite nucleic acid molecule according to the invention further comprises a non-digital data-encoding system block [SB] nucleic acid, wherein said [SB] nucleic acid is localized upstream and/or downstream of the [DB] nucleic acid.

As used herein, a “non-digital data-encoding system block [SB] nucleic acid” is intended to refer to a nucleic acid that allows the indexing, the provision of metadata, the provision of a security system, a system for proof-reading, to the flanking digital data-encoding [DB] nucleic acid.

In one embodiment, the [SB] nucleic acid is localized upstream of the [DB] nucleic acid, as illustrated by formula (IIa):

5′-[UP]-[SB]-[DB]-[DO]-3′ (IIa).

In one alternative embodiment, the [SB] nucleic acid is localized downstream of the [DB] nucleic acid, as illustrated by formula (IIb):

5′-[UP]-[DB]-[SB]-[DO]-3′ (IIb).

In another alternative embodiment, the [SB] nucleic acids are localized both upstream and downstream of the [DB] nucleic acid, as illustrated by formula (IIc):

5′-[UP]-[SB₁]-[DB]-[SB₂]-[DO]-3′ (IIc).

In the later embodiment, the [SB₁] and [SB₂] nucleic acids are either identical or distinct.

In certain embodiments, the [SB] represents a nucleic acid having a length of from about 0 to about 10⁵ nucleotides.

Within the scope of the instant invention, the expression “from about 0 nucleotide to about 10⁵ nucleotides” encompasses 0, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950 and 10³, 5×10³, 10⁴, 5×10⁴ and 10⁵ nucleotides.

It is understood that when [SB] nucleic acids are present, the [UP] and [DO] nucleic acids are solely representing a barcode-encoding nucleic acid.

In certain embodiments, one or more nucleic acid molecule(s) of formula (Ia) may constitute a sector (S). In some embodiments, up to about 10⁵ sectors (S) may be assembled into a double stranded, replicative, composite nucleic acid molecule so as to constitute a track (T). In some embodiments, up to 10⁹ tracks (T) may be pooled so as to constitute a Pool (P). In some embodiments, Pools (P) may be grouped so as to constitute an array (A). In some embodiments, the arrays (A) constitute a DNA drive. As used herein, the expression “DNA drive” refers to the physical support on which the digital data are stored.

It is understood that the [UP] and [DO] nucleic acids may allow to locate a sector (S) inside a track (T) or a pool (P) of tracks; and/or may allow the specific amplification of a given sector (S) from a given pool (P); and/or may allow providing a recognition site for the editing of sectors (S) in vitro or in vivo.

A device according to the invention may be characterized by its storage capacities expressed in octet (o), kilo octet (Ko) mega octet (Mo), giga octet (Go) or tera octet (To).

In some embodiments, the capacity of a device according to the invention is ranging from about 1 o (octet) to about 10⁵ To.

Within the scope of the instant invention, the expression “from about 1 o to about 10⁵ To” encompasses 1 o, 5 o, 10 o, 25 o, 50 o, 75 o, 1 Ko, 2 Ko, 3 Ko, 4 Ko, 5 Ko, 6 Ko, 7 Ko, 8 Ko, 9 Ko, 10 Ko, 50 Ko, 100 Ko, 250 Ko, 500 Ko, 750 Ko, 1 Mo, 5 Mo, 10 Mo, 25 Mo, 50 Mo, 75 Mo, 100 Mo, 150 Mo, 200 Mo, 250 Mo, 300 Mo, 400 Mo, 500 Mo, 600 Mo, 700 Mo, 800 Mo, 900 Mo, 1 Go, 2 Go, 3 Go, 4 Go, 5 Go, 10 Go, 15 Go, 20 Go, 25 Go, 50 Go, 75 Go, 100 Go, 150 Go, 200 Go, 250 Go, 300 Go, 400 Go, 500 Go, 600 Go, 700 Go, 800 Go, 900 Go, 1 To, 5 To, 10 To, 50 To, 100 To, 500 To, 10³ To, 5×10³ To, 10⁴ To, 5×10⁴ To and 10⁵ To.

As illustrated by FIG. 2, sectors (S) may be assembled into a track (T) that corresponds to a double stranded, replicative, composite nucleic acid molecule according to the invention; tracks (T) may be pooled in pools (P), which pools (P) can further be grouped in one array (A). One or more array(s) (A) constitute(s) a DNA drive.

The uses and methods according to the invention may be performed in vivo, in vitro, ex vivo.

One aspect of the invention relates to the use of a device comprising at least one double stranded, replicative, composite nucleic acid molecule comprising a nucleic acid of formula (I):

5′-([UP]-[DB]-[DO])ₓ-3′ (I),

wherein,
[UP] and [DO] represent a pair of non-digital data-encoding nucleic acids, each having a length of from about 0 nucleotide to about 10⁴ nucleotides, preferably from about 10 nucleotides to 200 nucleotides;
[DB] represents a digital data-encoding nucleic acid having a length of from about 8 nucleotides to about 10⁶ nucleotides, preferably from about 500 nucleotides to about 5,000 nucleotides;
x represents 1 to about 10⁵,

for the storing and/or the editing and/or the retrieving of digital data.

Another aspect of the invention relates to a method for storing digital data comprising the steps of:
a) assigning to said digital data at least one double stranded digital data-encoding [DB] nucleic acid sequence (S_{DB}) and at least one pair of non-digital-data-encoding [UP] and [DO] nucleic acid sequences (S_{UP}) and (S_{DO});
b) synthesizing the at least one nucleic acid of formula (Ia):

5′-([UP]-[DB]-[DO])-3′ (Ia),
from the sequences (S_{UP}), (S_{DB}) and (S_{DO}), respectively;
c) assembling the one or more nucleic acid(s) of formula (Ia) so as to obtain a double stranded, replicative, composite nucleic acid molecule comprising a nucleic acid of formula (I):

5′-([UP]-[DB]-[DO])ₓ-3′ (I),
wherein x represents 1 to about 105;
d) storing at least one pool comprising from 1 to about 10⁹ composite nucleic acid molecule(s) of distinct sequence and of formula (I) obtained at step c) into a storage cell.

In some embodiments, the digital data may be compressed and/or encrypted. In practice, the compression and/or the encrypting may be performed by any suitable algorithm. As used herein, the term “compression” is intended to refer to the action of encoding information by using fewer bits than the original representation, e.g. by eliminating redundancy. Non-limitative examples of algorithms for performing a compression of digital data may be LZMA (Lempel Ziv Markow Algorithm), LZMA2.

In practice, the step a) of assigning to said digital data at least one double stranded nucleic acid molecule, encoding both digital data and non-digital data, may be performed automatically by a suitable software. Illustratively, digital data, e.g. binary data may be assigned a particular nucleotide sequence.

Another object of the present invention is a computer software for implementing the use and method for storing digital data.

In one embodiment, the method of the invention is implemented with a microprocessor comprising a software configured to assign to digital data at least one double stranded nucleic acid molecule. In some embodiments, the software is configured to achieve a C+G percentage of from about 35% to about 65% for the sequence of the composite nucleic acid molecule according to the invention. In some embodiments, the software is configured to prevent that the sequence of the composite nucleic acid molecule according to the invention would encode one or more RNA(s), preferably would encode one or more mRNA(s). In some embodiments, the software is configured to prevent that the sequence of the composite nucleic acid molecule according to the invention would comprise one or more initiation codon(s), in particular in the [DB] nucleic acid. In some embodiments, the software is configured to achieve a sequence of the composite nucleic acid molecule according to the invention comprising one or more stop codon per 200 nucleotides in all 6 reading frames. In some embodiments, the software is configured to prevent that the sequence of the composite nucleic acid molecule according to the invention would comprise one or more specific restriction site(s). In some embodiments, the software is configured to prevent that the sequence of the composite nucleic acid molecule according to the invention would comprise one or more restriction site(s), in particular BamHI, BsaI, BbsI, EcoRI, FokI and I-SceI. In some embodiments, the software is configured to prevent that the sequence of the composite nucleic acid molecule according to the invention would comprise one or more repeat(s) of at least 4 identical nucleotides.

As illustrated by FIG. 1, each bit “0” may be assigned either nucleotide A or nucleotide C; and each bit “1” may be assigned either nucleotide G or nucleotide T.

The 256-bit digital data of formula (III) as follows: 0100000110010010101000010000110000001101010001100011001000000000001111 0111011010000011111001010001110100110101101101000011000000010000010011 1100001000101000001100010100100111111110100001110111100110000100011001 0100111110100010011111101111001100110111011000 (III);

may be assigned the 256-nucleotides sequence (S_{DB}) of formula (IV) as follows:

(SEQ ID NO: 1)
5′[CGCAACCGTCCGACTAGCTAAACGCAACGTCAACAAGTCTCGCAAGT
AACGTCCGACCCAACCCAAGTTGAGTTAGGAGAACCCGTTTGACGATACC
GGGCTCCTTCGAGTCTTATCAAAGTCCAACCCGCCCAAGAAGGTTCCAAG
CAAGAGACAAAGGCCCGCTACGAATTGGTTTGAGACAAGGTAGTTGCCGG
AACCTCAATTCCGATAAGTTGGCTCAAGACGGTTGGCTTTGACGGAAGTC
GTTAGGAAC]3′ (IV).

Pairs of indexes [UP] and [DO] may hence be added at the 5′ and the 3′ extremities, respectively.

For example, indexes of 25-nucleotides may correspond to the sequences:

(TATGAGGACGAATCTCCCGCTTATA; [UP]; SEQ ID NO: 2)
and
(GGTCTTGACAAACGTGTGCTTGTAC; [DO];. SEQ ID NO: 3)

Therefore, the resulting composite nucleic acid molecule of general formula (I) may be represented by the nucleic acid sequence of formula (V) below:

(SEQ ID NO: 4)
5′[TATGAGGACGAATCTCCCGCTTATA]-
[CGCAACCGTCCGACTAGCTAAACGCAACGTCAACAAGTCTCGCAAGTAA
CGTCCGACCCAACCCAAGTTGAGTTAGGAGAACCCGTTTGACGATACCGG
GCTCCTTCGAGTCTTATCAAAGTCCAACCCGCCCAAGAAGGTTCCAAGCA
AGAGACAAAGGCCCGCTACGAATTGGTTTGAGACAAGGTAGTTGCCGGAA
CCTCAATTCCGATAAGTTGGCTCAAGACGGTTGGCTTTGACGGAAGTCGT
TAGGAAC]-[GGTCTTGACAAACGTGTGCTTGTAC]3′ (V).

In practice, the step b) of synthesizing the at least one nucleic acid of formula (Ia) may be performed by any suitable method known in the state of the art. Non-limitative examples of suitable methods include chemical synthesis and enzymatic synthesis.

Illustratively, chemical synthesis of nucleic acid molecule may be performed up to about 200 nucleotides. Nucleic acid molecules with a length of up to 200 nucleotides may be assembled so as to obtain nucleic acid molecules of the desired length, e.g. up to about 10⁶ nucleotides.

In practice, step c) of assembling the one or more nucleic acid(s) of formula (Ia) so as to obtain a double stranded, replicative, composite nucleic acid molecule comprising a nucleic acid of formula (I) may be performed as for the assembly of the nucleic acid of formula (Ia).

In practice, the step d) comprises storing at least one pool comprising from 1 to about 10⁹ identical or distinct composite nucleic acid molecule(s) of formula (I) into a storage cell.

As used herein, “identical composite nucleic acid molecules” refers to composite nucleic acid molecules having sequences with 100% identity. As used herein, “distinct composite nucleic acid molecules” refers to composite nucleic acid molecules having sequences with less than 100% identity.

The term “identity” or “identical”, when used in a relationship between the sequences of two or more nucleic acids, refers to the degree of sequence relatedness between nucleic acids, as determined by the number of matches between strings of two or more nucleotides. “Identity” measures the percent of identical matches between the smaller of two or more sequences with gap alignments (if any) addressed by a particular mathematical model or computer program (i.e., “algorithms”). Identity of related nucleic acid sequences can be readily calculated by known methods.

In practice, the nucleic acid identity percentage may be determined using the CLUSTAL W software (version 1.83) the parameters being set as follows:
for slow/accurate alignments: (1) Gap Open Penalty: 15; (2) Gap Extension Penalty: 6.66; (3) Weight matrix: IUB;
for fast/approximate alignments: (4) K-tuple (word) size: 2; (5) Gap Penalty: 5; (6) No. of top diagonals: 5; (7) Window size: 4; (8) Scoring Method: PERCENT.

In some embodiments, the step d) comprises storing at least one pool comprising from 1 to about 10⁹ composite nucleic acid molecule(s) of distinct sequence and of formula (I) into a storage cell.

Within the scope of the invention, the expression “from 1 to about 10⁹ composite nucleic acid molecule(s)” encompasses 1, 2, 3, 4, 5, 6, 7, 8, 9, 10, 20, 30, 40, 50, 60, 70, 80, 90, 100, 200, 300, 400, 500, 600, 700, 800, 900, 10³, 5×10³, 10⁴, 5×10⁴, 10⁵, 5×10⁵, 10⁶, 5×10⁶, 10⁷, 5×10⁷, 10⁸, 5×10⁸ and 10⁹ composite nucleic acid molecule(s).

Within the scope of the invention, the expression “at least one pool” encompasses 1, 2, 3, 4, 5, 6, 7, 8, 9, 10, , 20, 30, 40, 50, 60, 70, 80, 90, 100, 200, 300, 400, 500, 600, 700, 800, 900, 10³, 5×10³, 10⁴, 5×10⁴, 10⁵, 5×10⁵, 10⁶ pool(s).

In practice, a storage cell may be any suitable recipient known in the state of the art to sustain the storage of nucleic acid molecules. In some embodiments, the storage cell may be selected in a group comprising a living organism, a glass-based recipient, a metal-based recipient, a silica-based recipient, a polymer-based recipient, a paper-based recipient.

In certain embodiments, the living organism may be a cell from a bacterium, a microalga, an archaeon, a fungus or a yeast. In some embodiments, the living organism is a particle, such as a phage or a virus. In some embodiments, the living organism is a prokaryote, in particular selected in a group comprising a bacterium, such as actinobacteria, chlamydiales, cyanobacteria, firmicutes, proteobacteria, spirochetes, thermotogales; an archaeon, such as an archaeon of the phylum Crenarchaeota, Euryarchaeota, Korarchaeota, Nanoarchaeota and Thaumarchaeota. In certain embodiments, the living organism is a eukaryote cell, in particular a cell selected in a group comprising a protozoan, an alga, a plant, a fungus and an animal cell.

In some embodiments, the animal or the animal cell is not a human or a human cell, respectively.

In some embodiments, the storage of composite nucleic acid molecules according to the invention may be performed in solution or in a dried state. In practice, the storage in solution of nucleic acid molecules according to the invention may be performed in an alkaline solution, in particular a solution of pH above 8. In practice, dried nucleic acid molecules according to the invention may be obtained e.g. by spray drying, spray freeze drying, air drying or lyophilization. In some embodiments, lyophilized nucleic acid molecules according to the invention may be further encapsulated under inert atmosphere.

In one embodiment, the storage of nucleic acid molecules according to the invention may be performed on paper, e.g. on FTA® cards (Whatman®).

In certain embodiments, the storage of composite nucleic acid molecules according to the invention may be performed at a temperature of from about −196° C. to about +100° C. In some embodiment, the storage may be performed in liquid nitrogen (about −196° C.). In some embodiments, the storage may be performed in a freezer, in particular at a temperature of from about −80° C. to about −20° C. In some embodiments, the storage may be performed at room temperature, in particular at a temperature of from about +15° C. to about +30° C.

Within the scope of the invention, the expression “from about −196° C. to about +100° C.” include −196° C., −180° C., −170° C., −160° C., −150° C., −140° C., −130° C., −120° C., −110° C., −100° C., −90° C., −80° C., −70° C., −60° C., −50° C., −40° C., −30° C., −20° C., −10° C., −5° C., 0° C., +4° C., +5° C., +10° C., +15° C., +20° C., +25° C., +30° C., +35° C., +40° C., +45° C., +50° C., +55° C., +60° C., +65° C., +70° C., +75° C., +80° C., +85° C., +90° C., +95° C. and +100° C.

In some embodiments, the method further comprises the step of:
e) organizing and grouping the pools obtained at step d) into at least one array comprising from 1 pool to about 10⁶ pools, preferably about 96 or about 384 pools.

Within the scope of the invention, the expression “from 1 pool to about 10⁶ pools” encompasses 1, 2, 3, 4, 5, 6, 7, 8, 9, 10, 20, 30, 40, 50, 60, 70, 80, 90, 100, 200, 300, 400, 500, 600, 700, 800, 900, 10², 10³, 10⁴, 10⁵ and 10⁶ pools.

Within the scope of the invention, the expression “about 96 or about 384 pools” encompasses 96, 102, 108, 114, 120, 126, 132, 138, 144, 150, 156, 162, 168, 174, 180, 186, 192, 198, 204, 210, 216, 222, 228, 234, 240, 246, 252, 258, 264, 270, 276, 282, 288, 294, 300, 306, 312, 318, 324, 330, 336, 342, 348, 354, 360, 366, 372, 378 and 384 pools.

In certain embodiments, the composite nucleic acid molecule obtained at step c) is a plasmid, a cosmid, a prokaryotic chromosome or a eukaryotic chromosome.

As used herein, the term “plasmid” refers to a small extra-genomic DNA molecule, most commonly found as circular double stranded DNA molecules that may be used as a cloning vector in molecular biology, to make and/or modify copies of DNA fragments up to about 50 kb (i.e. 50,000 base pairs (bp)).

Within the scope of the instant invention, the expression “up to about 50 kb” encompasses 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.2 kb, 2.4 kb, 2.6 kb, 2.8 kb, 3 kb, 3.2 kb, 3.4 kb, 3.6 kb, 3.8 kb, 4 kb, 4.2 kb, 4.4 kb, 4.6 kb, 4.8 kb, 5 kb, 5.2 kb, 5.4 kb, 5.6 kb, 5.8 kb, 6 kb, 6.2 kb, 6.4 kb, 6.8 kb, 7 kb, 7.5 kb, 8 kb, 8.5 kb, 9 kb, 9.5 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb and 50 kb.

As used herein, the term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid.

Cosmids often refer to DNA nucleic acid molecules ranging in size from about 32 kb to 52 kb.

Within the scope of the instant invention, the expression “from about 32 kb to 52 kb” encompasses 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, 51 kb and 52 kb.

As used herein, a “prokaryotic chromosome” refers to a nucleic acid molecule that can replicate in a prokaryote.

In some embodiments, the prokaryotic chromosome is a bacterial chromosome, preferably a bacterial artificial chromosome. As used herein, the expression “bacterial artificial chromosome” or “BAC” refers to an extra-genomic nucleic acid molecule based on a functional fertility plasmid that allows the even partition of said DNA nucleic acid molecules after division of the bacterial cell. BACs are typically used as cloning vector for DNA fragment ranging in size from about 50 kb to 350 kb.

Within the scope of the instant invention, the expression “from about 50 kb to 350 kb” encompasses 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 210 kb, 220 kb, 230 kb, 240 kb, 250 kb, 260 kb, 270 kb, 280 kb, 290 kb, 300 kb, 310 kb, 320 kb, 330 kb, 340 kb and 350 kb.

As used herein, a “eukaryotic chromosome” refers to a nucleic acid molecule that can replicate in a eukaryote.

In some embodiments, the method further comprises the steps of:
c1) amplifying in vivo the at least one composite nucleic acid molecule comprising a nucleic acid of formula (I) obtained at step c); and
c2) extracting and purifying the amplified composite nucleic acid molecule obtained at step c1).

In some embodiments, the step c1) is performed in vivo by a living organism, preferably a microorganism.

In some embodiments, when the storage and/or the amplification of composite nucleic acid molecules according to the invention is/are performed in a living organism, the said composite nucleic acid molecules are introduced inside said living organism, preferably in at least one cell of said living organism. In practice, these steps may be performed because the composite nucleic acid molecules according to the invention are biocompatible.

In practice, introduction of a nucleic acid molecule into a prokaryotic or eukaryotic cell may be performed by any suitable method from the state of the art.

Illustratively, introduction of a nucleic acid molecule into prokaryotic cells, in particular bacteria may be performed by transformation of competent bacteria or transduction using a phage. As used herein, the term “competent” refers to a bacterium that has been treated so as to increase its ability to uptake an extra genomic nucleic acid molecule into its cytoplasm. The skilled artisan is familiar with techniques for preparing competent bacteria.

Illustratively, introduction of a nucleic acid molecule into eukaryotic cells may be performed by transformation, conjugation, transfection or transduction using physical/chemical treatments, microbes, viral particles and/or liposomes.

In practice, one may refer to the manufacturer's instructions, when commercial kits or materials are used, and/or alternatively refer to the protocols described by Maniatis et al. (Molecular cloning: a laboratory manual. Cold Spring Harbor Laboratory, 1982).

Yet, another aspect of the invention relates to a method for retrieving digital data stored by a device according to the invention and/or stored by a method according to the invention, said method comprising the steps of:
a) sequencing at least one nucleic acid of formula (Ia) comprised in a double stranded, replicative, composite nucleic acid molecule comprising a nucleic acid of formula (I), so as to obtain at least one nucleic acid sequence (S_{UP}-S_{DB}-S_{DO});
b) converting the at least one nucleic acid sequence (S_{DB}) into digital data; wherein step a) is optionally preceded by step a0) of amplifying the at least one nucleic acid of formula (Ia).

In practice, the step a) of sequencing a nucleic acid molecule may be performed by any suitable technique known from a skilled in the art. Non-limitative examples of suitable sequencing techniques include the Sanger sequencing and the next-generation sequencing (NGS), otherwise referred to as the high-throughput sequencing (HTS).

In practice, the step b) of converting the at least one nucleic acid sequence (S_{DB}) into digital data may be performed automatically by a suitable software or in silico. The decoding step may be performed with the reverse approach than the coding step.

In practice, the optional step a0) of amplifying the nucleic acid molecules comprising nucleic acids of formula (I) may be performed in vivo in a living organism, or in vitro by any suitable techniques known from the state of the art. An example of suitable techniques to amplify a nucleic acid molecule includes a PCR. When PCR is performed, the (S_{DB}) may be amplified using a primer pair than advantageously hybridizes with complementary sequences within the 5′ (S_{UP}) sequence and the 3′ (S_{DO}) sequence. In practice, the step a0) may be performed in vivo because the composite nucleic acid molecules according to the invention are biocompatible.

Another object of the present invention is a computer software for implementing the use and method for retrieving digital data.

In one embodiment, the method of the invention is implemented with a microprocessor comprising a software configured to convert at least one nucleic acid sequence (S_{DB}) into digital data.

### BRIEF DESCRIPTION OF THE DRAWINGS

FIG. 1 is a scheme showing a strategy to encode a digital data file as a composite nucleic acid molecule according to the invention. The upper panel shows a 256-bit long digital data to be encoded. The lower panel shows the corresponding digital data-encoding nucleic acid, on the basis of a code wherein bit “0” is encoded by A or G nucleotide and bit “1” is encoded by C or T nucleotide (see [DB] of sequence SEQ ID NO: 1). The [DB] nucleic acid is flanked at the 5′ extremity with the [UP] nucleic acid of sequence SEQ ID NO: 2 and on the 3′ extremity with the [DO] nucleic acid of sequence SEQ ID NO: 3.

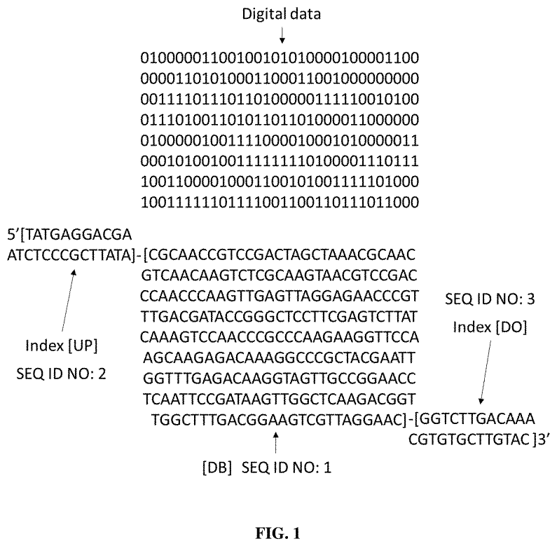

FIG. 2 is a scheme showing the organization of a DNA drive according to the invention. From the top to the bottom of the scheme are represented: (1) a sector (S) that corresponds to the smallest unit on which digital data are encoded; the sector (S) is made up of the nucleic acids [UP], [DB] and [DO]; (2) sectors (S) may be assembled into a track (T) that corresponds to a double stranded, replicative, composite nucleic acid molecule containing multiple sectors; the box represents a double stranded nucleic acid molecule comprising one or more origin(s) of replication. Tracks (T) may be pooled in pools (P), which can be assembled in one array (A). Several arrays (A) constitute a DNA drive.

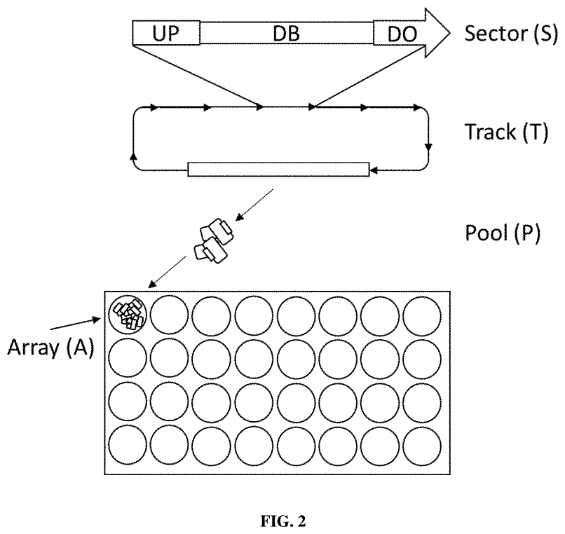

### EXAMPLES

The present invention is further illustrated by the following examples.

### Example 1

A DNA drive of 1 array (A) comprising 96 pools (P) of 10,000 tracks (T), each made of 9 sectors (S) consisting of one [DB] nucleic acid of 3,000 nucleotides flanked by a pair of [UP] and [DO] nucleic acids of 25 nucleotides each can contain the equivalent of 3.24 Go of digital data at an encoding density of 1 bit per nucleotide.

### Example 2

A DNA drive of 100 arrays each comprising 384 pools (P) of 10,000 tracks (T), each made of 9 sectors (S) consisting of one [DB] nucleic acid of 3,000 nucleotides flanked by a pair of [UP] and [DO] nucleic acids of 25 nucleotides each can contain the equivalent of 1.3 To of digital data at an encoding density of 1 bit per nucleotide.

### Example 3: Example of a DNA Drive Containing the ‘Declaration of the Rights of Man and of the Citizen from 1789’

A DNA drive was physically built so as to contain a single text file corresponding to the French Republic founding text of the Declaration of the Rights of Man and of the Citizen from 1789 (“La déclaration des droits de l'homme et du citoyen de 1789”), which is reproduced integrally hereunder (Source: Bibliothéque Nationale de France).

“Déclaration des Droits de l'Homme et du Citoyen de 1789

Les Représentants du Peuple Français, constitués en Assemblée Nationale, considérant que l'ignorance, l'oubli ou le mépris des droits de l'Homme sont les seules causes des malheurs publics et de la corruption des Gouvernements, ont résolu d'exposer, dans une Déclaration solennelle, les droits naturels, inaliénables et sacrés de l'Homme, afin que cette Déclaration, constamment présente á tous les Membres du corps social, leur rappelle sans cesse leurs droits et leurs devoirs; afin que les actes du pouvoir législatif et ceux du pouvoir exécutif, pouvant être á chaque instant comparés avec le but de toute institution politique, en soient plus respectés; afin que les réclamations des citoyens, fondées désormais sur des principes simples et incontestables, tournent toujours au maintien de la Constitution et au bonheur de tous.

En conséquence, l'Assemblée Nationale reconnaît et déclare, en présence et sous les auspices de l'Etre suprême, les droits suivants de l'Homme et du Citoyen.

Art. ler. Les hommes naissent et demeurent libres et égaux en droits. Les distinctions sociales ne peuvent être fondées que sur l'utilité commune.

Art. 2. Le but de toute association politique est la conservation des droits naturels et imprescriptibles de l'Homme. Ces droits sont la liberté, la propriété, la sûreté, et la résistance á l'oppression.

Art. 3. Le principe de toute Souveraineté réside essentiellement dans la Nation. Nul corps, nul individu ne peut exercer d'autorité qui n'en émane expressément.

Art. 4. La liberté consiste á pouvoir faire tout ce qui ne nuit pas á autrui: ainsi, l'exercice des droits naturels de chaque homme n'a de bornes que celles qui assurent aux autres Membres de la Société la jouissance de ces mêmes droits. Ces bornes ne peuvent être déterminées que par la Loi.

Art. 5 La Loi n'a le droit de défendre que les actions nuisibles á la Société. Tout ce qui n'est pas défendu par la Loi ne peut être empêchê, et nul ne peut être contraint á faire ce qu'elle n'ordonne pas.

Art. 6. La Loi est l'expression de la volonté génërale. Tous les Citoyens ont droit de concourir personnellement, ou par leurs Représentants, á sa formation. Elle doit être la même pour tous, soit qu'elle protége, soit qu'elle punisse. Tous les Citoyens étant égaux á ses yeux sont également admissibles á toutes dignités, places et emplois publics, selon leur capacité, et sans autre distinction que celle de leurs vertus et de leurs talents.

Art. 7. Nul homme ne peut être accusé, arrëté ni détenu que dans les cas déterminés par la Loi, et selon les formes qu'elle a prescrites. Ceux qui sollicitent, expédient, exécutent ou font exécuter des ordres arbitraires, doivent être punis; mais tout citoyen appelé ou saisi en vertu de la Loi doit obéir á l'instant: il se rend coupable par la résistance.

Art. 8. La Loi ne doit établir que des peines strictement et évidemment nécessaires, et nul ne peut être puni qu'en vertu d'une Loi établie et promulguée antérieurement au délit, et légalement appliquée.

Art. 9. Tout homme étant présumé innocent jusqu'á ce qu'il ait été déclaré coupable, s'il est jugé indispensable de l'arrêter, toute rigueur qui ne serait pas nécessaire pour s'assurer de sa personne doit être sévérement réprimée par la loi.

Art. 10. Nul ne doit être inquiété pour ses opinions, même religieuses, pourvu que leur manifestation ne trouble pas l'ordre public établi par la Loi.

Art. 11. La libre communication des pensées et des opinions est un des droits les plus précieux de l'Homme: tout Citoyen peut donc parler, écrire, imprimer librement, sauf á rëpondre de l'abus de cette liberté dans les cas déterminés par la Loi.

Art. 12. La garantie des droits de l'Homme et du Citoyen nécessite une force publique: cette force est donc instituée pour l'avantage de tous, et non pour l'utilité particuliére de ceux auxquels elle est confiée.

Art. 13. Pour l'entretien de la force publique, et pour les dépenses d'administration, une contribution commune est indispensable: elle doit être également répartie entre tous les citoyens, en raison de leurs facultés.

Art. 14. Tous les Citoyens ont le droit de constater, par eux-mëmes ou par leurs représentants, la nécessité de la contribution publique, de la consentir librement, d'en suivre l'emploi, et d'en déterminer la quotité, l'assiette, le recouvrement et la durée.

Art. 15. La Société a le droit de demander compte á tout Agent public de son administration.

Art. 16. Toute Société dans laquelle la garantie des Droits n'est pas assurée, ni la separation des Pouvoirs déterminée, n'a point de Constitution.

Art. 17. La propriété étant un droit inviolable et sacré, nul ne peut en être privé, si ce n'est lorsque la nécessité publique, légalement constatée, l'exige évidemment, et sous la condition d'une juste et préalable indemnité.”

The text file was encoded using the ISO8859-1 standard (commonly referred to as Latin-1) and has a final size of 5,253 octets. The file was compressed with the Lempel-Ziv-Markov chain Algorithm (LZMA). The compressed file (binary provided hereunder) has a length of 2,293 octets. 0101110100000000000000001000000000000000000000000010001000111010010010 0001100110110000110010101010110001000011000111010010101110110000100101 1011101001100110111011111010011000000000001011111000101001010100001110 1011111011100101000001101100001011110110001010011110110000101011100000 1100101000001000100010010001110110101101010010100110001001010101001100 1011101101110011111011100110011111010010100000011101101000011010101100 1000001011010101001100100101000011011000100100110001111111101101010111 1010101111111000011111111110011110100100000110101110101100000000010101 0001110110011100010100011100100010011111100010110010100001110000011110 1011010001100111111001111100010011000100111011001110111110000110001011 1100011010101001111011011010011101101011111101101110111000111010000100 1000011000001110110110110101011110111000010001110000011010010110101110 1010100011000110000001111101011011100111110100011010001111001101001111 1000110001111010001100110011110110001011101010111110010110011011010110 0111000011111101100011111110110001110011101110111101001111010011011110 0100011100001010001101101110100101111010001011101000101101001010100011 0110001011100011111100001000001011011110000001011100101011010111110010 0000011111010010011010110001000101000111010110001001001111001001001101 0000111110101011111110100000000001101100100011100101101001000010010001 0011001011011111000000100000111010000000011000010110011101011110111110 0011100100100010111101100111100100010001010100100101111110000000010011 1011111001111010110010000001110011001001011000111100101000010100001110 0101100110001000000001101110111001100101001101001100101110010010000101 1101000111101110011010110110001110011101010010111111110100010110111001 0011100111000110000100010111010111011111010111000101101100000110001110 0000111100101111101000001110001100111101101001110010011000110101000000 1001110101100101010101101101010110100010000101001010101011111011110001 1000111011010000110011011100001100111000101001111101100001011111011101 0000110001010011100010110011100000000101110101111111110000011111110011 0101010001100111011110111011111000011000111110001011110111100101101101 1100101110110110010100000110110100011001100000011001001100011110110100 0011101011000011010111100111100111101011111100111001111111101101111001 0001001101111101110110101011000000011101001101001011010110100111000101 1111110000011000010011011000011011110000010111111100100100001111111000 0111001101101000000000100010100001101000010110110000000000100011101101 1001110010000001011111011011011100110100000011011111000101010011100100 1110010001011010101011010010100110011110100010001111100010010101001010 0010110100001101010011001111110011111100110011011101011011011100101011 1001111010011100100111000011001010010100000101100000010110010100000001 1101111000011011100011000000000101001110100001100111100000010101110010 1101101000110111010000001100010001101111010101101111101100010010011001 1110111110010101100011011000010101110001111010100110010100000000010101 0001010011001110111000010100001001100010011111011000010110111101011010 1111111111001010000111100011110001011110001000100100111010011000111010 0111000101101111110111001101011001111101111101100110110100000010011101 0111100100001110000011001111100011111110101110011111001000101110010011 0011100101110111011001111100001100100101110010001101010101011001001010 0010010001001001101011110101101100111011110001100001001111011011011111 0101001010000010100111100111100101110001101110111101110010101000111101 0100010010100011111000010010011011100110100001000000110110001010100101 1000010010010000101100110111011111010100000010111100100101101001001010 0100110111001010011000011110001101001001000010101110110100111001111101 0101000001100100101010000100001100000011010100011000110010000000000011 1101110110100000111110010100011101001101011011010000110000000100000100 1111000010001010000011000101001001111111101000011101111001100001000110 0101001111101000100111111011110011001101110110000111100100000011101110 1111010110100000101110111100100111011101001000100110111101101010011110 1110011011111001010111010111100101011100110010100010100110101100011100 0000100111110011100011101111100010111001011010100101000100110111100110 1110111010100011000001011010110000110110010000001011100110111010000100 1011011001101100010111010101010011110101111001110100001110101100011110 1001100010000001001001011000110010001100111110111000111001000011000101 1110010010101010011100000100010001000011010111010100011100001110011010 1010111100000010011010101111100000110001110011100011110110010111111010 1000111011011111011110110001011101011100001101100100010110100010011110 0101001011110100011100111110100011000100010100100100001100001001000101 1011100000000111001001100101011111101001011000101010101101001010101001 0100110101001110001001010011100011001011001110001101010100001111010011 0001111011000111111011001000111010110100000011100010111111001011101010 0110110010010011011110100010000100010010101101111000111010010110000011 0010111000100011010010110001011110100001101101010011110100111110100001 0111111110010100101011100111101110111001110000000111100011100101010111 0100110000001001110011101100000000011110101110010111001011010000111000 1101001010011010000110100000111000010001011011100111100100110101010101 1010110011000011010111000100010100101111000101111101001110001001011000 0110101110010011011010011111010011000110111000011001111000110101101000 0011000000010111000001111010100011101101000000110111011111111011001111 1101110100000011000101110110011100101011110110110010011001101110100101 0001110111010110100110010101111110100111100010011110101010000111101000 1000100000110111111111000010010011001111111100001110100011001110011011 0000101010010001001101011000011001000100010100000010000101101110111001 0000111101001010111010000111101000010001100000001000010001110010011001 0111001100011111000111101000110011010001011110110101111101001111111000 0011010001111101010111100111100001100110110000100111110010100101000011 0110110110100010011001111000100000101110100010011000010001011001100100 0000000011010001010100100011111100110111100110001000111010111100000100 0101100110110010111101111100101001000110001001001000001111110001110100 0011000010101101101001111000011000000010100111111110110100011100011101 1111000111010101110111010110000001110110100001001000001011100101011111 1111100001011001111110000100110110101000110101100110001101111011011101 1011110010000100001110101001110110111110110100111001111101111011110110 0011110111011110000000010111100000001101000100101010111100011101100100 1111100010110011101000100010101101100011001110011100000000101100111110 1010111100011010100010111111111100000110000010100100001111100111100001 1000101110011001110001111000000100001100000001010000011101110001011001 0110001101001011001100101100111101010111100100100100111111001101101110 1010110001001001110100110001010001000010010100100001011110110001111101 0000100000001010000101001101001011000000001101011001110110000101100011 0101011000011100000110101100110001101000001010000000000000011100010000 1100000110011000001110001000111101110111100101100011000010110011101111 0011011010110101110010101011111001010010010000000101010110110001100011 1111111001000101001100110111001100111000111101011100000111011110000110 0111111011100111001010000100111010111101001001111011010010000010000011 1111011101001110010101111110011111110101001010100101000111000111010001 0100011110011001001101001100011100111111100100010110001110010010100011 0001010011011000001001110000001001011010011110000000000100110011100110 0000101101110101111100011110110010011110100011110100100100010001001001 1101110101001010111011001110110111001010001100011100001000101000001100 1000111000010100111100011000111111101000100100100111100100001011011010 0001111111001100111111011011100001101111100111011101101010101000001001 1101010001000010010111110011000111111111010000111100001101110010111010 1000101000101011010011101100100110011000101111100110110010110011101010 0001000110001001101110010000011010010001100100001100100111101110010110 0101001011010100110010111001001010000011101011010000111100010011000010 0001100011100000000010010101101000011000101110011000111010110111001110 0100101001110001011000101101101011001110011111001100011001011100100000 0101011000000111110010011100001101001111011100110101010101100100000011 1110101011000000000010110110101100010100101100111001000011010001111101 1000100111011011010001001110000111001000010000110101000001010001100100 0011010101110100101000000010101110100010010101010111111001010111100101 1000011001111100001101110000101000000010101000110110001110010010001100 0111010110011001101001111000001001100101111111100100110000100100111011 0011011100111000110101000110000100110001001111110010000110111010010101 0101110001001000111011111110101101010110100001111001110110010111001110 0110100000110111100000001101110100100010110110110100001101011001101100 1001100100010010010100001111000011101010101001111101110010110011101001 0000001101010100010000001101000100000011001101110111101010001001000100 1110100101100000010111100100001100111111000000011010100011100000111100 0000111100001011101110011011100110110101011111110101011101110010101111 0100111010110111001110100011101111100001001110011110101010100100011100 1100011011111001101100001110111101100001010100110101010110100110000001 0100110010001001101111111101000101000110110110011101000111110001000010 0010101011101000001111111011101101001100001111010001000000010010011111 1101011101111001111000101001110001101111010001111101001100111010101100 0010011010001110110010010001110101100011010011010110000000101010010011 0011110000000011111010001101100110111001011011011001110111101111001101 1001100100010001101011111100110110001011010011010100111100101000100000 1101010000001000001110111100001000110110000001101110111100000000110111 1110011000001101101110100110011010001010000110000000001001110111100110 1111110011110011100001110011111010000101001010110000000111011101110011 0010100111001000110010110001011100010110010011100000010001100001101001 0101110101010101001011100011101110100110010110011001100011101101111100 0111011011011110011100100101110101101111010110111100110110111110010000 1011110101011001011000000011101100011011110110001101010010011110101100 0111110001011110100110111000101100011000010101011000100110000001110011 1101111110011010000011000100010011111100111010101111010100011101011011 0101001001110011001100001011010101000101101000101010000000001100111111 0101011101000000101000110110010011000111000110110100001001101000011001 0000000010001011101100111001100101100110001001100101001011001110100111 1001101110101111001011110111100010001110110100110100110011110100110101 1101111111011110111100101010010111100011001011010101111001001101101010 1110110001010010100111101110100100100011100010100101001100010100000011 0111101101001010101101001011110111011011100101110101110000110010011010 0010101110001111001001110001011101010110111010110010000111001010100101 1101000001001001011011101010000010011001110110101000111100000001000010 0011010101101001001011111011110000011000110100111110110111001010010001 1100110010101010100001110101111001111110001110101100000001010111100000 0111001000000110111101000111101101110110000100111010001111000110000010 1001110010110111110101101111001000011011101011110010011000110101101111 1000111010001111010000101011010110101111100111011000100010111001011101 0010100100010001110110001110101010100001111000010111000001010000100001 1000011001010010110110000000001011011110110110100110110001110110100101 1101111011000010100111000100011000110011000010010011110010001011010001 1110001011111011101111111010001100110000100110100110100100011101110111 0100100111100010011110010010010000000000111111001101011001101011000110 0001011101001111101000101011011110111110110111010001000001001110011000 1111000000100100110111001000001101001100101011100010000001000010000110 1011010110110110111011011100010111111000110001001000001110010100011001 1110010101000011010100000111100010110111000111111100101001100101010011 0010101010000101100010100110101100101101101100011010010010110010110000 0110000001011100011111000100111111000001101111101101010001011101111000 1111000011111011111110101110100001000000011111110101010111011110101011 1110001100001100001100111010001110001000000011111100011111100001111110 1001001110010101000001101010101000110001010101100110001100101011100100 1010010011011001010001110100101111111010111111111110100111110001111011 0001011101000011000001001111001100010111001011011100010010111111011010 0111011110100010100110010101001111000010000111011011000100100110011001 0100010000100010000101101000111000100111001010110010100000111011011110 0110101100111010001011100111001101000001001110011001101100100111000100 0010110001110001111110000111011100101101001010110111100011101110101101 0010011100000110100000010000110001011001101001001100101110100110001011 0100101101111100110111100111100101110010101110101101000110010010100100 0110001110100000111111001001001101100100110011100000101110110000111100 0100000010100001000100111011101100110111000110100011000000110010110111 1101111111100110010001000010010000000000100000110101011010100011101001 0100001000010100001111001111000101000000000010110000000011001001001111 0100001111101011000101111101011011000001011101010100001001010001100101 0000011001110111110101011000010101011100010010010010011010001011100011 0001101111010110100100100011010110000110111011000100110001011010101101 1000001100111110001001101100110011100110111011011110000000100000001100 1110011011110111000011101100000001001111100100011011110111010100110100 0011101000000110000100010010010111010001000001001000010110000011011100 1110100100100101010000010111010001101001100011111001110100111110110110 0111101110000001011000100001011010100100111101100000011001010110100010 0111001101111101111010011000000011101010111000101100011101111010011011 1111011010010110010011100111110100010111101101010100111010110101111101 0100110010100100100010010011000011101011011110000101110101110010011000 1010110101111110011010001110101001001101111111000001001110101001111001 1111001001110100100111110000110110111000100001010001010100000000000011 0001011110010000001111001100101110001011111001101111101110000010100000 0000111110000101101011110100110010100101111011000100100000011101100111 1011101100010001101001100011100011011110010010001111001001100010101010 1010101001001111100111010101110110000111010100101001001100001000011111 0000101101000000110010110101001110110000100010101011001111011001100000 0111101110010110001110110101011111010100001100011010111100111000111100 0001001100110110000010111010111100000000000101111000111100001001110010 0001011001001111011011011001001111101111101110001010010101111000000000 0000111011010010010110010110110011011000100101011001001100000110100010 0100010011111111101110100110100011010000100001101100001001110010010110 0011011001100100111000000010001101010101111010101010100111001000101110 1111100110100111111011100010000011100111010000010111011101010110011001 1111011010001000001011110100001001110101100110111101100111111100100111 0111001000111110001110011000111111011010000010001111001111111011100110 1011110010101010000111101011001001010101010111110110011110011011011001 0011111111100111000000100110011101001000111101111000010101001000100011 1101000001111001100111100001111110000000000011001111101010011110010000 1110110011101000000101011101100101011110010101010100010100011101010001 0010010100111111111100010100010110110100010010101001010110010011010000 1100010011001011010000001000001010000111011100000010111000100001010010 1101000100110100001110000001010001111110101101000111110111110000100101 1010101011000011110100111101110011010001010101111011001110111001010010 0010110111111010001100000100011100010010011101100000110100000000011111 0001110101100011101000000110110000111111101111001111000010100100100110 0100101100100010000000100101100101100001111101111100101001100010101000 1100010000100001110110101101100110100011110101001111010100101000100001 0111111001100011111011000101101001101010001111111101100111011101110100 1111101110111000110110001100010010101111000000011110101011011001111000 1111011010101111011010010000110100100110100000010111111100010101100101 1011011011011110011100111110100101101110000110001100000011100001100010 1000000001001101010010111001010110100110011100000101011101100000110101 0101101101101000001101000000001100000100100111100010110101100110010111 1111111110101101100001001101010011100100010010011101111100111010100100 1111000101100110011101010110011101000110011101001001000101111001000101 1011001011111111110000100111001010001001101001111111101111001100000010 1010010110110001100100010101011111000101101100000100010011010101100011 0100010000011101100110010011011001111111110011011100010100010111010100 1100111011001001011011101100010101011010111011010000111101101100000011 1000001101101100111100100101000011100000100111011111110101000000011101 0100100010001011001001011010111000001010011011000011001100011000110111 0010111100100110110010110100001101111001100001100000010101011010100011 0001110000010000001001001101111111101010100000011001000100110011110000 1001010110001101000001001011011111111110110010010011100010000000100111 0101110011100010010011100010011001101001110000111110011000111100111110 0111101000100000111001010001001011110000100110101000011111010000111001 1001011100010101111100101111010010101100110111110111011111100100001011 1111110100001010001101101100011110010101001101001101101011100111101110 0100101110110001101011000010001110100010011000011110110111111110000101 1111101110011011010010010111100101001000101011101100001101011000101011 1011100000000000101101011001001011000010100010001001110000100001010100 0111010001111001001001000100110111100011000111000001000101101010000011 1011100000001100011110111100010101111100001111010010010101110111110101 0101000111011111101101110011111111111010110001010110111011111101110110 0101010111111001000101001111001001011101000011111000010010000101111111 1111000111111111010001100010101111111011001001101110100010000011011000 0100000101101100101000100100111111110110100101010100111110110111000110 0111111001010010010101110010010001001110000111010011101100100010001010 1111000010110101001011111110001111010001100110101001110011011101011110 1001111011111100011000110001011100101001110111110011011110011011110000 1111011111010001100000101011000101110111101110111001100101001011000110 1111101001101111000111100011100101100000111110111111000011110111011110 0001100011001101001111010110010001010011110100101011100110111111111100 0110111110011110111001011110000011111111110101001011100100110111100000 0000

This binary file was converted to nucleotides using the Church-Gao-Kosuri encoding scheme (Church et al.; 2012, Science, Volume 337, Issue 6102, pp 1628), in which A and C are represented by bit 0 and, T and G are represented by bit 1. For each bit (0 or 1), the corresponding nucleotide was attributed randomly one of the two possible nucleotides (A or C for 0; T or G for 1). The resulting sequence of 18,344 nucleotides was divided into 6 data blocks ([DB]) of 3,000 nucleotides and 1 data block of 344 nucleotides. Then, each [DB] has undergone cycles of random nucleotide modifications in order to allow convergence of the sequence towards a biocompatible sequence that follows the specifications of the DNA drive: controlled G+C percentage (between 35% and 65%), no encoding of mRNA, no initiation codon, at least one stop codon every 200 nucleotides in all 6 reading frames, no restriction site for the enzymes BamHI, BsaI, BbsI, EcoRI, FokI and I-SceI, and no repetition of more than 3 identical nucleotides.

The resulting nucleotide sequences are called data blocks [DB] ([DB-1] through [DB-7]) as depicted in Table 3.

TABLE 3
Nucleotide sequences of data blocks DB1 through DB7

| SEQ | | |
|---|---|---|
| ID | DB | |
| NO: | NO: | Nucleotide sequence (5′ to 3′) |
| 5 | [DB-1] | ctcttgcgccaacaaacaacaaccgccaacaacaacaaccaacaaccaacgaaagaacg |
| | | ggagccgacgccaattaaggaggcaaagtcctctagctcggaactaaccggaccggtatc |
| | | cgctatttcggccaatcctagtaggtagaaggacgtcttgcgttggctaaggcaacaacaaa |
| | | ctcgggttacctatacgcgctcaaattgcgcttggtcggtaagcgccaacggattcaacgag |
| | | gttagtaacgcgaagggtcggcaacgatctttcccaaggaagctaccaagaacgccctaa |
| | | gaacgttcttataggcgctcctcgccggaaataatctatcgacggcctatttcggcgggaatt |
| | | gggctttccttccttgttcgacgagccaaacttgcggctaaacgtagctattcctccaactagt |
| | | agcgcgacgtactacgctccaattcttcaagcctacggcaaggttgtttcgtagagattgga |
| | | gcgattgggttacccttgagggttactttgcgcctaccaagtataggtcgcggacaacaacc |
| | | tcgctaacggtagtacgggaactagacctttaagccctacttgtttcccgcttacgcgaacctt |
| | | gccaacttgtatcgtcgaccggaaggtttgccttggtacctaaggaccgaaggtcttacggt |
| | | ctttggacccgtcaagagtttcccgtatagctccgggtattaggataaggtagtcgcttggttc |
| | | gtctttcgggaaaggtcgcaacgaagaaccttacaaagggcgtcggaggctagcttgtcttt |
| | | acaatcccggtcaaacggcgactagtctattgcgctagcaaggcaaggaaacccttgggc |
| | | gcttaggtacttgttataccgtcgaccgttgccttatccggtttcccgtaaagttgctaccttac |
| | | ggcctttgcttacctcggtagcgctttggacgattacgtaggataggccttgaaacggttgga |
| | | ggaacggttgggcttaccgttacggtctttcgtttctacttggcgccacgtttactcccttgacc |
| | | ctataacggcttaggtcgcctcgtttctcccgcttgataccgattctacgagagaaaggagga |
| | | acgagggcaaggtttgacaagccaacgaggcgtttcaacaatcggtcctagattcgcgttg |
| | | gcctaacaacgttggagcctacgtctaggcaagaaagagcccgggctattacctactccttg |
| | | gaatccgccgtagacaattgggcgatattggtttagacccaaacccttcttaagcaagggac |
| | | gagtagcctaacctacgcccgaaggaatcttcggttgacaaacgcaaacgttcgcaacaac |
| | | cttccaatcggaagggctcgttgagggttcaagttcctacgaacgagtttcggacgggtaag |
| | | ccctacctatatacgacgcgtttggccaaaccctaaggtattgggcctttgagaggaagcaa |
| | | accgggccttaagcctagtaaagggtccgcgacaagatacaatttacgagtccggaccgc |
| | | caaacccgtatttctttacgtcctagccttctacggactcgttcctacgaccctcttgctaacgg |
| | | gtctttaaggagagtagtaccggtaagggagataagcggttggttctaccgaggagggaat |
| | | ccttgacggtaccttaccctacctcttgctagttcgttgtagattgccctcttcggcaaccttcc |
| | | ctttcccaagggtactatttggcgaacccgttaaaggccgttgagtctaattgccgccttcaa |
| | | ggagagcccaactaaggtatcggacgagatagcggcttctcgagtcgaactcaactagac |
| | | gagctcgagggttcgggtcaagtcaattgcgtatcaacggacggagggcaaaggaattga |
| | | actagacgggttcggcaacgatttgtagttagacccttaccgctactttccctcaccgggaac |
| | | ccaaagaggtagcggttggttgaaaccttgggttacggagcgagcaaggaagttatttgag |
| | | ggcggttgacccttaccgtttgaaagattggcgttgcctcggcgtatttactctttcgtcgtccg |
| | | atcccaaggcggctaaattaaggcaaaccggacgccggaaattgtagtatcaacgggcgc |
| | | ttaaccggatattgccttggaattgtcgcgtttgtcctttccgttggttgcttcttggactcccga |
| | | cttcgtttgctttaggctagaggccaacaaggtagaaggctaagaggagcttcgccttgacc |
| | | gcttgttgtcaaccggccaataaggcggaaccttattggcaacctcttgttgtccgcctacaat |
| | | ttgggtaaccggtaattcggcgaccaacaaagccctagccaaggatacaagaggcggac |
| | | aacaacaagaccttgcgtcttacgggaatcaaccctcttgggcttcttcttgacttagccaacc |
| | | gtcggtttcaagagagacttgaatacggtccgaccgcggagatatagtatcctatacggacg |
| | | tttcgccctcccgttgtaactccgctagaagatcaatcggagccaaggcgataaggaaggg |
| | | ttgaagggttgccttaaggcggtataggcgtcggtaatctcttgccttgtctacgggactccg |
| | | ggacaattaagataagcgccaaagcgtaaaccctcgtccgagaacaaccggtcgtttacaa |
| | | gtcgttaccggacccaaaccgagaagttcgcaaaggccttgtaaccaagagcttgacgctt |
| | | agtagaccgtcgttctaccaacgtcaagcccgtattggagagcttcggtttaggaacgcctc |
| | | cttacttggaggtttacgcgagtaacgtaggacaatcgcgggaacgttgagagaaggaatc |
| | | gccaaacaacgagcgaacgataaggacttgcttgacaagagcaacgaaggcaagccgg |
| | | gttagtcaacgcggagg |
| 6 | [DB-2] | ggatcggagcgttggtttgtccgctacccgtttaacgggtaacgagttgaaagaacgccga |
| | | cggtataaggcaagttctacgggaccgattcgttggtagggacttagcttccgggttcttggt |
| | | attacttcggcgaaacccgactttctagggtcctaaccgttcccaaggcctttggaacgttgg |
| | | gtagattgaagggttaagaacgcgggactaattacggtccgagggcgggattaagttgtaa |
| | | ccttccgcctagttaagcccgtctatagagcggccgactatacctccgaaatactaaggcgc |
| | | gttgagcgtattacttgagtttcccggcaaagcctttgcgtagtcggtttagagccgcgacca |
| | | atagaaggttacttggcctaggtaaaggagggagggtatttaagcgctcccgtttcgctccct |
| | | aatcgcaattggtcaacgactaattcgggacggctaaactcaaaccgtcgtccctagcgac |
| | | gcttaacctacgacgacaataggccgtattgcgagtcgcgaacaaagaggttacgcctctt |
| | | agactactagacgccgtctttaagcgacggcaaattggaccgtagacgactacaagagcg |
| | | ggagtagacttgaagggttagagcgcaaccgtccgactagctaaacgcaacgtcaacaag |
| | | tctcgcaagtaacgtccgacccaacccaagttgagttaggagaacccgtttgacgataccg |
| | | ggctccttcgagtcttatcaaagtccaacccgcccaagaaggttccaagcaagagacaaag |
| | | gcccgctacgaattggtttgagacaaggtagttgccggaacctcaattccgataagttggct |
| | | caagacggttggctttgacggaagtcgttaggaaccgggtactcaacccttgaggtagtttct |
| | | aggctcccaagcggtatttgacgcctttcggtctaagaactctttcggttcgtcgagccgttta |
| | | gttacttctttggcctagcgggatattggaagctcgttaaggccgataccgagaaggcgagg |
| | | aaagggcaaacctacgttggaaggtcaagttaggttgaccgattgacgcttatatcctctccc |
| | | gacgtagggtacggcttgaggtcgataccttcccaatcgtagaggaaccgtcggactaaca |
| | | acgagttaaggcgttcgccaagcctcttaggaaggaggaaagcttgcgctctatccggtta |
| | | gattgtccgttagccaagggataggcaagttgctccttacctcaaccctacgaagaggaaa |
| | | ggcctaccttacggtttcggtaacgggaagcaacggacctagttgactactcgctctccgttc |
| | | caactacctccctccaattctcggtctagaacgttcaacgggccggagatctcttgtaaaccc |
| | | taattcgagattgggcaaccggaacttgaattgcccgggtaggaagcgtttgtcgagaaag |
| | | gtcggaggttgagggtattcaagcttgagcgggaaccttaggacgaaatcgtcgccctaag |
| | | ttgactctccgaggttatcaagggaagttggcgaacttccctaactatcctacgacccgtcca |
| | | atacgaaatcggagttcccaacaattgccgacgtaagatcggttgtcgaagcgtccctctct |
| | | ctagtctcctatagctcctcgccttctatccgttaactcctcgacttgaccgtacgcttaagggc |
| | | aagtcgctatacccgggtagacttaacgggtcttaacgttggtcggcctcaatttctattcgca |
| | | aaccggtaacgagttgttccgcgttctctccttcgtcctactaaggcttggcgaacgaaacta |
| | | acgccgcgattcttggaacttgcgaagagtaccaattacgcgggaactcaaggcgaatag |
| | | gcaagcgttgatacccgtcgtagctaagtttcgaaggttgcgcaactcgttgttggactagcc |
| | | gctatttccgtttcggtcgggactttcccaaacttggaaagggaatctcgagggcgaagtcc |
| | | aaactaagggacttgcggaccaacaaagggtcgcggtaatctttcctcttatcaacggtccc |
| | | ttatcctagccttagacccttctcccaagggcaactcaatcttcgttccggttccgacttcgatc |
| | | tagcgtatcttccgtaaccggcgcttgccctaactagactattggcaagcggttgagaatttc |
| | | ccgacgcgtacccgtctatttaagaaggaggagccggttgcgaaggaaaggagggaaac |
| | | gtaagggtcaagtagagtctacaacggaccaaactcgggaccaagtttcgctacctttcttc |
| | | gaccaaaggcggtattgttggtaggccttgttgattgagaaacccggacctcggtaggactt |
| | | tacgagcgttgcttagtccgccgtaattagttagaagctaccgggcgggagattctaaggaa |
| | | gagcgggtttagaagggtaacgacgttgagagatacaagggtctaccgacctccaacggc |
| | | ggttggtttcaaagccgccgtacttgttggtaaacttgctcaaggacgggccttagtaccctat |
| | | agaagcaatccttagaggacccggaagaacgcaatagacaaccgaaacgaggcgttagg |
| | | tactaaccgggtcgacgctcgttatcaacttggagcaacgaacggcaaacaagacccgaa |
| | | cggtccgaattaagcttgacggaacgggttcccgttgataacgtccgtctcaatagttgcgtc |
| | | tagttggcgccttggtttacaaaggcgaaaggttgctatctttgccgggtacccttaattcgga |
| | | acctccttggtaatcgcctctacccggattcgtagtagccctccttaatttgcccgccaactctt |
| | | gctacctccttaaactcaagcggccgtccgaccaaaccaattagcaagcgctacgaacgttt |
| | | gtaaggcgttgccgtcaaga |
| 7 | [DB-3] | aattgagattggaccaagaaagcggaaggaggaatcgggtagttgtaagagccgaaagta |
| | | ccgaagaagcccaagttggtcaagggctacaaggcaactagagtcttagccgggtcaaag |
| | | gccaaacctcgacttgttggtagtctaacgggcaattgattggtaccggtcgctagttaggga |
| | | gcttcccaaattgaggctaccctaagcaaacgcggtaagctcttgggttgggacaatcgga |
| | | ctttgggcaacgacttagtatcgaccggatagtacgtaacgtcttggcttcgggagtaggtta |
| | | cgaaactacccggtagctactttcgtaggtttcgtctccggtccggtttcgtttcttgtattccctt |
| | | ggcggtcgttgccaaccaatcttggccaacccgtataaatccgctcgatttgacctttcggcc |
| | | taagttgtacctaggacgttagaacgacctcgcgtagtaaaggacgggaaggtccaacaac |
| | | gcttacggttgagctcgttgcccttatagaacgcggttgttgttcccaattcaacctcgcctaa |
| | | cctttgtccggttcaacggaaatcgggacttacgggaacggttacccaagaaccgtccaaa |
| | | ccgataaaccgttagttacctcggccgagtcaattatccgaggacggactattccgggtcgc |
| | | tcttggaatacgccgaagttgttacggcggagttctagagtaaagcctaagggctaagtccc |
| | | tctaccgaaccgacgatcctaaacgcgtttcggaaagttggcgaaactccaaacctctcaaa |
| | | gagccgtcgaagaggcaaaccaagtagcttaaggtcggccaataggaccttcgctagtcc |
| | | aagggaaaccggagattaaggaccttctaacccgctaccaacaacaacaaggtaccgaaa |
| | | cggaacccttccgtcaaacttgaacgcaaggttagggagggtccgattaacggcaacgatt |
| | | acttgcttgtaaggcggagcgtctagggactctatagttggacgagcctccgcaaacaaga |
| | | gctattattaccggaccgggtttggtcctcaatcgacgtacggaggtaaggaattgcccttgg |
| | | agcgggaaaccgttagtttacccgtacgggtttcgttccttgcctagcaaataaggtcgcggt |
| | | tatacgccgttgcttagaagccaacgaccaattgggtagggcgacgggacgatagtttgtc |
| | | cgttgttgctatcctctcgactatcccgttaacgggctaactataccgttgacggacgaagga |
| | | tccgtaaagggccgttgttgactcaagcggcaatttccgaagcgcaagtaacgagaaggc |
| | | gtcaacctacttgaaacccgaagaggagccgttgacaacccaaagaaggaattgccggcc |
| | | caagcttcgttcgcttgttaacttgtagtccgacttggcgaccttgtatccgaataactcccga |
| | | cgaaggtcgttcgatactcgagttcggccgggattagttcctctcaaggaaagggcaacga |
| | | actagccaaagtacgcaaggtaccctagaattggcaaggaacgggtttgctaccgacgcct |
| | | aattggccgacaataggaggctaaacgttgttgccgtccgggtttattcgttccaaggctttg |
| | | gcctttattgcttcgctcgcgaacaatccgttctataacgcaacgcctattgggaaggcccgg |
| | | tttgtttcgccaatttgccaagtcggtcctcttgatcgaacgctaactatcggagacgttcgtcc |
| | | gccttaaggaacgcttgttaaggattccgcggaaggtatatccaagaaaggcccgacgtctt |
| | | gcctaacccttatcctaccttccgacaattcctccttggcttgacgattacgagaagattcgag |
| | | ccgtccgaggtactaatctaaaccgggcgagtcgcaaagtttaacgccttaccctacccgg |
| | | aaatttcaaccaaccgaagcgcgtcgaaccttaaatctttccggcccttgctagtcggtacgt |
| | | tcctcctctcctttacctcttaccgcttaggctagtacgggacgttggaagtaaaggacgcgg |
| | | gcctacaaacgctattacccaagttggactccgggcaacttagaagggtcgttaaggcgct |
| | | agagcggaagaaccaagttggatagcggacccaaccaagattattcgaggaaagcgcct |
| | | agtccgttcctccaaggcgaaatttggaggaccgacttgattcggagcaagaagttacaag |
| | | ggccgacaagacaattcgctcaacctcgaacgtcctaaccgtctctcttgctactctaccaac |
| | | cgatagggctccctaatatcgagaggtttgcctctcgtttcctcttaaccggaagggttacaa |
| | | ggcttgaaccgataacccaagagctaccggcgtaccgttccgaataaattaccgggatcgg |
| | | acgtccttcgaattgtccaactccggcctatttgttggcctccgtaaacgacgaattgcgtccg |
| | | gattgactttcccggagatcaagtccaagacggcaatacgttgggcctcaacttaggtcgcc |
| | | gagctatctttcccgactaacttgattggttgcgcgtcgatagtatccaattggactttcggcct |
| | | agggacgttccgtataccaaggcttggacccaacttattgagcctaccgcttcgtaggctaa |
| | | ccttctaggccggcttaagacttaagaacgccgacgcgacaagtttacccttgcgctctctaa |
| | | ggttgcggtaagaggaattgagcctaaacccggcgctataacgacccaagtctaactaccc |
| | | aattccggcttgcgttgctcgcaatcctaactaattgagccgaggaaccaagattggccgac |
| | | ccttccggtttgaaaccaaggagctaacgttccaacttggcccaaagttgaaactcgggag |
| | | ggccgtagggaagtagtatct |
| 8 | [DB-4] | cgttgttgcgataggtattgaagcgagggtagaagggctattagttacgggcgaacgggatt |
| | | tggaccctcctttacttggcgctctagaatcaagggccgtaccttctttgtccttcggaaccttg |
| | | cttgtcgtcaactcgctacttagatctcggcgacggaccaactataagtaagccctaaggcg |
| | | tttgggtcgcaatataacggcttaggcctttcgccctttggacctacaagcaagagagaggg |
| | | atcaaacggttgggatttcggatccggacaattgtatccctaaccaacgacgacgttgttgcg |
| | | cgttagggtcctttgaacgcgaagggaaaggattggcgaccttgggctaattactttcgcgc |
| | | gtaacctacttcgaccttgagtacgccgaaattgctcggaccttctacgtatcgtaacaaccg |
| | | ctctacgacggccgtttcaaacaacgttggctaccgtagtccgtaggtacgagtaggagga |
| | | agggaggttagggtaattcttccggacgaaatcaagtagcgttgggccttagtcccgattcg |
| | | aaggagctacttgtccgctacctccaacttagctccaacctaacccgggattggcaaataac |
| | | gtaggccaaacgtcttgagttgacccaaacgtagttgggacggaacctttcggctttagaag |
| | | gaattagcccgctacaaggccaaacaacgacgggcgtttacgtagttgttccgttgacgttc |
| | | aaagggacttggtcgccaatctactcgcggaaacaacttgcggtcggtaagtaagcgacg |
| | | gtaagaaagtcctcttccctagttcaagcggcctaattgaccaacgaacgtacaattcgacg |
| | | ctagggagatatagcgcctagggaaagggcgggataattccgattccgtacggcaagttc |
| | | gtcttgttcccgggcttcttagtttacggtcctacgcgttagagtcgttgcgagtcggttccttat |
| | | tcggtttccgccaagagggtctataggactagtacaacccggtcggcccttctttgcttaaag |
| | | gctctcctccttggcgcgtaactttgtcaagcgttgcgaagtaggtcaagaggaaaggcaac |
| | | gctagcgtaactccgtccaaacgttacttgtcggtttgacggagaaaccgtaacgacctcctt |
| | | gggtaagttcgatcttggctagcccttgctagtcgtctctcctcctttacgtaaggacaagcgt |
| | | agcgctaactaggctacctctctccaaaccaaggaattgggtagcgaggtatccaacctctc |
| | | aagtcggaataaggcccgttcccgtattatccaagacgtctaaccggactaccaacccgaa |
| | | ctagggcggaattgacgtaatattccgtcccgccttaatcgactcgtacgggatacggttaa |
| | | gtcttgctcgggtaatcttggcggttccctaactttcttcgccgtagaagtacgggtcgaagg |
| | | cgagggatttgggtattggaggttacgctagccgattgtcaaggccgcttatagattggccta |
| | | cttaggctagattgcggaactcgaatcgacgtttcggtagccgaagaaagggaacgcgcct |
| | | ataagtacctcgaaacccttcgttgagtagcctctcgaggataagcggttattgcgtcggga |
| | | agagttctagttacccgtacgccgtatccctatctttaacgggtaagccttgaccgcggtaga |
| | | tagtagggatcgtactcaaagggaagcgctcctcgttagaaaccgacgaagcggcttgcta |
| | | tacaacgccgtactttaggcgatcccgtttcaaacccgcaaagcaagtctcgcttagcctaat |
| | | ctttggcttggccaacgtaacggctacttgggcgtctttactcgcctaaattgccggcctctct |
| | | atctcaacgggagcttggaattgggtaacttgctaggcaaaccctagaggttcaacaaggtc |
| | | cgaccaacggagggtatcaagttgagtaggtaggcaaagaagggcgaacggttaacgga |
| | | acaagagccggtaagcgtcttggtatcttcgttgcctcaacgtcggtctagttgactaagtac |
| | | cgtcgaggcgtttgcccttgagaacggttagaacctctcgtcgagtagaggtttaaggtcgg |
| | | aaagaaatcggtccgcggtatactctactaccgaaaggtcgtcccggtagctagctaaactt |
| | | ggaaactcgggcccaatctccaagacaagtacaaggcctagcctcggcttaaaccaacct |
| | | cggagttgcttaggctacttcgtcaaggtaggctaatcgggcgttgattaaacgctacggtac |
| | | cgaaagtaccggacgtaacctactccgtttacgccctagtcgaaagggtaacgcttgggag |
| | | ttcgttgttgcgcccttacgtcaaagacggataaggagaagccctttcggtagttcgcctaag |
| | | ttgaactccgttgcctacgactaaccaaccaaggttgtccgtctcttaaggctcggaacttca |
| | | actcgggagacttgttcgaacgatattcgttgcgggttcggctttagaacgaaaccgaaggg |
| | | acggaacgtttaaaccctcctccttctttccgccaaagtcgaaggactcgctttaacgcccaa |
| | | ataaccgccaaggatcggcgcgtagtattagttcgtcttgccctatttgggaacttccctccg |
| | | caacctttacgctaccttacttggacgagagccaagtagagccaacggttacctcttagggc |
| | | ccggtttgtaatataaggcctcgctaaggaagagcgcgaaccgcttaaatctccttagcggc |
| | | ctcttcttattaccggcgactacgcggaagaggacaaattcccaactcgttcaattgggccc |
| | | gactttggtccaacttagttggagtctagacctaggtattggcccggttaaccgggttagggtt |
| | | gtctagggaga |
| 9 | [DB-5] | accgaaaccaattggtttctagagcggtagggtagctagggttaacttacccgtcaacggcc |
| | | gttagcccgttaactacaaaccgttgggacctttgggcaacttgttgctacgactttaagctcg |
| | | aacccttcgagagagcaagtacctagctcggaagtaacggcctagatttactcctcgccga |
| | | agtaggaagatcccggtcgacgcgttgtttctaggtttgggttgctccttgggacctttgagta |
| | | acgagttcgaaccggaccaatacgttgccgtacctctttaatagtcttgaccgcctcggttgg |
| | | cttagacggtcggttagcaatatccgtcctctagaagggtacccgaaacttgcgtagtaacg |
| | | cctaaggacgtcctagacctaaccgaaagaccctaggagcaaggtacctaaggtcctatag |
| | | tccgagccaaaggtcgtagttgacttagaggccgggatcaagagggaagggacggatca |
| | | aactacttgaaggccttcggccgaattgacctcaaatattcccgggaccgttgggaaccttg |
| | | ctttccgagtctccgctagtcgttgaacttgagggatcttctactccgggaaaccgtcgcaaa |
| | | ccgacaaggcaataggacttctaatccgtcctagggagccttcaagagtcgacgagtcttg |
| | | gtaattcttggacgtttacgcggtacgagcgttcgcttatcaaggcctccgatactaacttaaa |
| | | gggctcaaccggttgtcctaatccggagtactaagtacgggcaaactcggtaggacccgtt |
| | | gaaagaacaaagcgacccgaaagacttgcgttattccggctttcccttctcaagtcaaaccg |
| | | tccgcttctttggaggttgttgaaggcctccctcaactcctaaccaaacaagaccaaggagc |
| | | gaggagagaaattgcgaagcgaccctaaccgagaaacttgtacgggtcaatcgcccaacc |
| | | aactattacccaaccgtcctcctacttggctacaagggttagcgtcaagatttggcgcttagg |
| | | aaacctcttgctagagcaaagaagctcccggaagatccaaaggacggtagggttctagctt |
| | | caaatcgcgcttgacctaataagccgacggagccctcggtaaagtcaattcgggtagctta |
| | | gaagccgcaaggagattacccttcgttagtccctccttcccgattcgagaggattacaacgg |
| | | aattgggaccgaagtaggccggaagttacggagttcgtcgtttcaaaccctcccaaacttac |
| | | gggacgtattgtagggacccgggattccaaccctaattgttactcccggcgttgagttagag |
| | | ccttagacaaggtcgccaaacggaccctaactaagacgagttagcccgccaactaataccc |
| | | tagtaacccttcggtaaggtcgccgactccgatatcaaacgcgttagaaaggcgacggcaa |
| | | ttggtacggtctacgttgtcttaggacggttcgggaacaacgaggaaagacaataggcgct |
| | | aagccgttgagtcccaacgtaagagaggagcaagacttgaaggcttggtcgggtctaagg |
| | | caaaccctttagcgattgcccgaggaacggtagttgatccgtaggttggagtctccgattaat |
| | | ccgttccggtttctccctcggttcgtcgagataagggcgattagaggtttagcgccgtaagct |
| | | aatccgaacgactacttccaattgctcggcggttacaagcttgctcgttacgacggcaagat |
| | | agtcgagggtttacgtataccgggagctacgccttcttggtttccaaatccggtcgctaaggg |
| | | tacggtttaatccggtagccgacttggtaaccttattcgggaacgacaagcgcaagcgatcc |
| | | caaacaaaccgtaactcgggtaatcaacaagttgccgtccgctttacctcgttgtaagtcgg |
| | | gttagggcccaatctaccaaccaagggttaacctcttctattggagacggccgataagctttg |
| | | cggccctcctaaccaattgagtacgttgcgttcttcccgcccttatacgtacctttcccttcttgt |
| | | acgccgaccgttgacgaagtacctctagcgctagctatccgaattgttacgggctataggtc |
| | | gtaaccgggatcgaagctcctaattccaagaccctttggcaacgattctaccaacggacga |
| | | ggagctacgttcttacaagaacgcgcgaggaaggttaggccttaaacaaggttcggtacga |
| | | ttcccgggattctagcggtttagctccaaggcccttagagggtcctttaacgtttaccaatccg |
| | | gacgtcggacaactattgcgattggacccaaccaactcttggcaattggaaactaagggcc |
| | | gacaataggcctacgggtagtaggagtaagacggtttcgttggcggtaactagacgcgagt |
| | | tgacaacccaaacaaggtagtctacgaagcggactcttcttccggagtcccgactagattac |
| | | gaaggacaaagtcgcaagcctcaagccttgggttggagttctaaggagcccgtataaacg |
| | | caaaggaggaaacgacttgaagccgaggaaaggcggaagtaatacttgaccaaccgcaa |
| | | gtcgctctattgtcgctagctagaaggtcctaccgagggcgtttgaattagacgtttggcggt |
| | | caagcaaacgggacgggagaaacctcttgcgactctagtacttccttgggagtcgaacga |
| | | acaagcttgtagaacctacggtagcggccttagggtattacgttgttgacgaagggcggtac |
| | | tcccttgttaacttgaaggcccggttggcttctaccaatcaagggtacgttgggtcttgacgg |
| | | ctaggttaatcgcgctccaattggcgcgtcctaatatagctctattgggaggacggttacgtat |
| | | tcggcctaaggttgggttccttgaaca |
| 10 | [DB-6] | ccgaaggccgggagaagcaattggagttgaccctatctccgcaagacctttgcgcaacctt |
| | | gtaaggacgttgaaacggttgtacccaaacaacggccggttgatctaagggtaatccaagg |
| | | gcttacgggagcaacaagctagttcggactagagggtcctctagcgataaagctaacgttc |
| | | gagaacgccgccgctaattgttgttgtcaagctaccgaggcttcgcaatacgatcgaagcta |
| | | gtccgacggctcaaaggaaagccgtaatcgtcgcccaactaccaatcgaaccttgaggtac |
| | | ccaagaggtcaataccctatcctaggcgaacgaaggatacaagggacccaatagcaaggt |
| | | tggatagtcgcccttggtattgggccaagaagaggcgatatcgtaaccgttgatccgttgag |
| | | ttacttcgaccgctctaggttcttacggtcggtactcgcctaaagcggcttgtttcgaccttaaa |
| | | ccgaacgggcaagacgaattgaggaacaaggctcccaaccaaggttgaacgttagcggc |
| | | aattgatccaaccgtcgtaaccttgttgtagggtccgtttaaccgctaagcctaaggaagccg |
| | | agtcctaaataaaccaagccgcggactaggcaacgggttagttgtccgataattaccgagc |
| | | gaccggaccgcaaatacaagttagtagaggcggacgtctaacgttgctcgaattggatcga |
| | | ctcgcaagaccctagttgggacttcccttgggattacctcttcgccttctagcaaggttgttgc |
| | | ggacggtagttaggtcgacttggtcgttcttgaccttcttcccgtccctaagctagggtaacca |
| | | aaggttcgcgattcgtccgggtaaatttgcgtagcgcgttgcttctaataaccggatcctccg |
| | | gatacccaatcgttggttaactctcgtccgattaggcgtcggcgtttacgttaagggttagaa |
| | | gaggcgggcaaaggaacttaacaacgttacccggaaagcgaccaaaccgaagtcgatcc |
| | | tcttgacgagaggagaaggaatttccaaagagcgggcttcaaacggctctagcggcttcgt |
| | | ctaaaccttcgcaaccaacttcccaagacgacttggaacgcgtagagtaaggccgctttgtt |
| | | gttgggatcttcttccaatccggatatccggtcctaccgaagaaggtattggtaagggatcgc |
| | | ctccgttgaccgattacttacttgatcgcggccgttagcaagtccggtatacgcctcaatagtt |
| | | tacgcaagcggaggaagcttggttgggtaaccgaagggaagctccctacttagacgggttt |
| | | ggagtttacgtccaaacgctcgcctcttattaccggaagccctcgatctttggaccgcgtagg |
| | | acaactacctacgtcgctcttaaattcgaccgacaacgttcgtaaggcctacggcttccttgg |
| | | ttgttacggcgttcaatagcaagcgttagcgaaggccgggagtaataataggagggagtca |
| | | agagagattatcggtcggagccaagttgagtcggacccaagttaaaccgtcgtcgtacggt |
| | | taatcctctaaccggtaaacctccgttcgttgggtcgatccaaacctttctctcctaccgaccg |
| | | cttaagcctagtcgaggtaccaagctaagtagtcaacggacgtaacttcccgtcgttacgctt |
| | | ggactacttcttaagcggcgacaaggattggacgtccaagtacccaatctcgagtagctcaa |
| | | ttaccgggccaactacccaagactaaggcgggttgttcgagctaaccaaggactcccgac |
| | | gtaattgtacccgccgatcgtaaattcgcccaataagcggattgggtttgtcggactccgac |
| | | gttaacgaacccaataagggatagttaagggcccgacgaagttaaagccggacggctaag |
| | | gtacaattgttacggcccggttcctttgtccgtttctacctcaaccttgccgcgaacgactcgtt |
| | | taaccgaaggctagaaactttggcgaaacggtacttacgagggacctagctttgtaagcggt |
| | | tatccgcgcggacttatttggaggtcgttgggcctaacctcttggttgtagacccgataccgt |
| | | cggcttcaattggaagatcgaaggctccttagtatcgggaagggtagttcctacgcgttagg |
| | | aaaggctcttaaacgcccttgagcaagccgtcaacgtttagtcgttgttggacccgaggttgt |
| | | aggtaagtaggatacgactcgggtccgcgaagaaatataggtcggcaacttagagtcccg |
| | | agctttagggaacaacaaacctcggctattcctacgcggaaacgctacctaaagaagggca |
| | | acgacaagcgcgaacggtataacgggtacgactactcccgacttaggttaccttaacgggc |
| | | aaccgaacgcttagctcaaacgttcggtcccaaccttcaattgtcgggtccctctagggttac |
| | | aatttgctccgcctctagggaggtttcgctatagcaagttcgttggtaggcttgacgggtttgg |
| | | ttgcgaggcaatataggaggtattgggtctttaggacgagcgagtttggccgcaatcgaattt |
| | | gacgcctagggcgaaccggtttaaccgaagcaactattggttgggttcaagggttgggtcta |
| | | ccggaaatcgagttgggtcgtactccggagttctcaagccaacttaggcaactcaaacgatt |
| | | cgtccgctaactccgacttggttgtcgtataagcgctctcctttggagtctttaacgtccgggtt |
| | | tacgagactacgcgattgcctcctaaagcctttaaccggtagccgggcttaagcccgaaag |
| | | agctttgaaacgcggatatccgcttgttgtcaattgtcgaccggccttagctaagggacgga |
| | | gggctagtttatacgggtcgg |
| 11 | [DB-7] | ggttcccggaacttcaatcgttccgctcctttaggttgaaggcgtttacggcgggtaaccgttt |
| | | agggttagcaaggcaacctcgaggaaataggtcgggtattgagggaagtactagaagctt |
| | | cccggagggttagaaggagggtaacggttaacgggacgaggaccaatttgtcggttgtca |
| | | acggttagttcgggtcaaaggaaaggacggatactttgctcggccgacctagaagttgaga |
| | | agagaggtccggaggttgggtttcaattaggttgccgtttagggcctagggtaacaaggttt |
| | | ggtttcgagccgcttgactacggatttgaccaaacca |

In practice, each sequence was scanned for the presence of forbidden nucleotide patterns (e.g., such as the presence of the BamiHI restriction site ‘GGATCC’) and one randomly chosen nucleotide within the pattern was altered into its binary equivalent. After multiple combinatory iterations the sequences finally converged into biocompatible sequences that follow the specifications of the DNA DRIVE (see Table 3).

For each [DB]P non-digital data encoding blocks [UP] and [DO] were appended before and after the [DB]. The sequence of the seven pairs of [UP] and [DO] blocks are provided in Table 4 and Table 5, respectively.

TABLE 4
Nucleotide sequences of the [UP] blocks

| SEQ | | |
|---|---|---|
| ID | | Nucleotide sequence |
| NO: | UP NO: | (5′ to 3′) |
| 12 | [UP-1] | tatgaggacgaatctcccgcttatac |
| 13 | [UP-2] | gtttatcgggcgtggtgctcgcatag |
| 14 | [UP-3] | tagtagttcagacgccgttaagcgcc |
| 15 | [UP-4] | ggggttccgttttacattccaggaaa |
| 16 | [UP-5] | ctgacgtgtgaggcgctagagcatag |
| 17 | [UP-6] | gaggtctttcatgcgtatagtcacat |
| 18 | [UP-7] | ggtaactgcgcatagttggctctata |

TABLE 5
Nucleotide sequences of the [DO] blocks

| SEQ | | |
|---|---|---|
| ID | | Nucleotide sequence |
| NO: | DO NO: | (5′ to 3′) |
| 19 | [DO-1] | aggtcttgacaaacgtgtgcttgtac |
| 20 | [DO-2] | accgatgttgacggactaatcctgac |
| 21 | [DO-3] | accgtacctagatacactcaatttgt |
| 22 | [DO-4] | atatcccgtgaagcttgagtggaatc |
| 23 | [DO-5] | aggtatggcacgcctaatctggacac |
| 24 | [DO-6] | agattcaatatgtgtcgtctatcctc |
| 25 | [DO-7] | agcgtttaaggtcacatcgcatgaat |

The sectors were synthesized chemically and assembled to obtain a final sequence of formula I: 5′-([UP]-[DB]-[DO])ₓ-3′ with x=7 (SEQ ID NO: 26). This sequence of 18,732 nucleotides was inserted into a replicative plasmid for manipulation of the DNA sequence in the bacterium Escherichia coli.

The plasmid was replicated in E. coli, extracted and sequenced using a DNA sequencer. The nucleotide sequence of the seven [DB] obtained experimentally was converted to binary file using the Church-Gao-Kosuri decoding scheme (A=C=0, G=T=1), the binary file was uncompressed with the LZMA algorithm and the text file could be recovered at 100%.

(full sequence of the DNA drive)
SEQ ID NO: 26
tatgaggacgaatctcccgcttatacctcttgcgccaacaaacaacaaccgccaacaacaacaaccaacaaccaacgaaaga
acgggagccgacgccaattaaggaggcaaagtcctctagctcggaactaaccggaccggtatccgctatttcggccaatcct
agtaggtagaaggacgtcttgcgttggctaaggcaacaacaaactcgggttacctatacgcgctcaaattgcgcttggtcgg
taagcgccaacggattcaacgaggttagtaacgcgaagggtcggcaacgatctttcccaaggaagctaccaagaacgcccta
agaacgttcttataggcgctcctcgccggaaataatctatcgacggcctatttcggcgggaattgggctttccttccttgtt
cgacgagccaaacttgcggctaaacgtagctattcctccaactagtagcgcgacgtactacgctccaattcttcaagcctac
ggcaaggttgtttcgtagagattggagcgattgggttacccttgttgggttactttgcgcctaccaagtataggtcgcggac
aacaacctcgctaacggtagtacgggaactagacctttaagccctacttgtttcccgcttacgcgaaccttgccaacttgta
tcgtcgaccggaaggtttgccttggtacctaaggaccgaaggtcttacggtctttggacccgtcaagagtttcccgtatagc
tccgggtattaggataaggtagtcgcttggttcgtctttcgggaaaggtcgcaacgaagaaccttacaaagggcgtcggagg
ctagcttgtctttacaatcccggtcaaacggcgactagtctattgcgctagcaaggcaaggaaacccttgggcgcttaggta
cttgttataccgtcgaccgttgccttatccggtttcccgtaaagttgctaccttacggcctttgcttacctcggtagcgctt
tggacgattacgtaggataggccttgaaacggttggaggaacggttgggcttaccgttacggtctttcgtttctacttggcg
ccttcgtttactcccttgaccctataacggcttaggtcgcctcgtttctcccgcttgataccgattctacgagagaaaggag
gaacgagggcaaggtttgacaagccaacgaggcgtttcaacaatcggtcctagattcgcgttggcctaacaacgttggagcc
tacgtctaggcaagaaagagcccgggctattacctactccttggaatccgccgtagacaattgggcgatattggtttagacc
caaacccttcttaagcaagggacgagtagcctaacctacgcccgaaggaatcttcggttgacaaacgcaaacgttcgcaaca
accttccaatcggaagggctcgttgagggttcaagttcctacgaacgagtttcggacgggtaagccctacctatatacgacg
cgtttggccaaaccctaaggtattgggcctttgagaggaagcaaaccgggccttaagcctagtaaagggtccgcgacaagat
acaatttacgagtccggaccgccaaacccgtatttctttacgtcctagccttctacggactcgttcctacgaccctcttgct
aacgggtctttaaggagagtagtaccggtaagggagataagcggttggttctaccgaggagggaatccttgacggtacctta
ccctacctcttgctagttcgttgtagattgccctcttcggcaaccttccctttcccaagggtactatttggcgaacccgtta
aaggccgttgagtctaattgccgccttcaaggagagcccaactaaggtatcggacgagatagcggcttctcgagtcgaactc
aactagacgagctcgagggttcgggtcaagtcaattgcgtatcaacggacggagggcaaaggaattgaactagacgggttcg
gcaacgatttgtagttagacccttaccgctactttccctcttccgggaacccaaagaggtagcggttggttgaaaccttggg
ttacggagcgagcaaggaagttatttgagggcggttgacccttaccgtttgaaagattggcgttgcctcggcgtatttactc
tttcgtcgtccgatcccaaggcggctaaattaaggcaaaccggacgccggaaattgtagtatcaacgggcgcttaaccggat
ctttgccttggaattgtcgcgtttgtcctttccgttggttgcttcttggactcccgacttcgtttgctttaggctagaggcc
aacaaggtagaaggctaagaggagcttcgccttgaccgcttgttgtcaaccggccaataaggcggaaccttattggcaacct
cttgttgtccgcctacaatttgggtaaccggtaattcggcgaccaacaaagccctagccaaggatacaagaggcggacaaca
acaagaccttgcgtcttacgggaatcaaccctcttgggcttcttcttgacttagccaaccgtcggtttcaagagagacttga
atacggtccgaccgcggagatatagtatcctatacggacgtttcgccctcccgttgtaactccgctagaagatcaatcggag
ccaaggcgataaggaagggttgaagggttgccttaaggcggtataggcgtcggtaatctcttgccttgtctacgggactccg
ggacaattaagataagcgccaaagcgtaaaccctcgtccgagaacaaccggtcgtttacaagtcgttaccggacccaaaccg
agaagttcgcaaaggccagtaaccaagagcttgacgcttagtagaccgtcgttctaccaacgtcaagcccgtattggagagc
ttcggtttaggaacgcctccttacttggaggtttacgcgagtaacgtaggacaatcgcgggaacgttgagagaaggaatcgc
caaacaacgagcgaacgataaggacttgcttgacaagagcaacgaaggcaagccgggttagtcaacgcggaggaggtcttga
caaacgtgtgcttgtacaagggtttatcgggcgtggtgctcgcatagggatcggagcgttggtttgtccgctacccgtttaa
cgggtaacgagttgaaagaacgccgacggtataaggcaagttctacgggaccgattcgttggtagggacttagcttccgggt
tcttggtattacttcggcgaaacccgactttctagggtcctaaccgttcccaaggcctttggaacgttgggtagattgaagg
gttaagaacgcgggactaattacggtccgagggcgggattaagttgtaaccttccgcctagttaagcccgtctatagagcgg
ccgactatacctccgaaatactaaggcgcgttgagcgtattacttgagtttcccggcaaagcctttgcgtagtcggtttaga
gccgcgaccaatagaaggttacttggcctaggtaaaggagggagggtatttaagcgctcccgtttcgctccctaatcgcaat
tggtcaacgactaattcgggacggctaaactcaaaccgtcgtccctagcgacgcttaacctacgacgacaataggccgtatt
gcgttgtcgcgaacaaagaggttacgcctcttagactactagacgccgtattaagcgacggcaaattggaccgtagacgact
acaagagcgggagtagacttgaagggttagagcgcaaccgtccgactagctaaacgcaacgtcaacaagtctcgcaagtaac
gtccgacccaacccaagttgagttaggagaacccgtttgacgataccgggctccttcgagtcttatcaaagtccaacccgcc
caagaaggttccaagcaagagacaaaggcccgctacgaattggtttgagacaaggtagttgccggaacctcaattccgataa
gttggctcaagacggttggctttgacggaagtcgttaggaaccgggtactcaacccttgaggtagtttctaggctcccaagc
ggtatttgacgcctttcggtctaagaactccttcggttcgtcgagccgtttagttacttctttggcctagcgggatattgga
agctcgttaaggccgataccgagaaggcgaggaaagggcaaacctacgttggaaggtcaagttaggttgaccgattgacgct
tatatcctctcccgacgtagggtacggcttgaggtcgataccttcccaatcgtagaggaaccgtcggactaacaacgagtta
aggcgttcgccaagcctcttaggaaggaggaaagcttgcgctctatccggttagattgtccgttagccaagggataggcaag
ttgctccttacctcaaccctacgaagaggaaaggcctaccttacggtttcggtaacgggaagcaacggacctagttgactac
tcgctctccgttccaactacctccctccaattctcggtctagaacgttcaacgggccggagatctcttgtaaaccctaattc
gagattgggcaaccggaacttgaattgcccgggtaggaagcgtttgtcgagaaaggtcggaggttgagggtattcaagcttg
agcgggaaccttaggacgaaatcgtcgccctaagttgactctccgaggttatcaagggaagttggcgaacttccctaactat
cctacgacccgtccaatacgaaatcggagttcccaacaattgccgacgtaagatcggttgtcgaagcgtccctctctctagt
ctcctatagctcctcgccttctatccgttaactcctcgacttgaccgtacgcttaagggcaagtcgctatacccgggtagac
ttaacgggtcttaacgttggtcggcctcaatttctattcgcaaaccggtaacgagttgttccgcgttctctccttcgtccta
ctaaggcttggcgaacgaaactaacgccgcgattcttggaacttgcgaagagtaccaattacgcgggaactcaaggcgaata
ggcaagcgttgatacccgtcgtagctaagtttcgaaggttgcgcaactcgttgttggactagccgctatttccgtttcggtc
gggactttcccaaacttggaaagggaatctcgagggcgaagtccaaactaagggacttgcggaccaacaaagggtcgcggta
atctttcctcttatcaacggtcccttatcctagccttagacccttctcccaagggcaactcaatcttcgttccggttccgac
ttcgatctagcgtatcttccgtaaccggcgcttgccctaactagactattggcaagcggttgagaatttcccgacgcgtacc
cgtctatttaagaaggaggagccggttgcgaaggaaaggagggaaacgtaagggtcaagtagagtctacaacggaccaaact
cgggaccaagtttcgctacctttcttcgaccaaaggcggtattgttggtaggccttgttgattgagaaacccggacctcggt
aggactttacgagcgttgcttagtccgccgtaattagttagaagctaccgggcgggagattctaaggaagagcgggtttaga
agggtaacgacgttgagagatacaagggtctaccgacctccaacggcggttggtttcaaagccgccgtacttgttggtaaac
ttgctcaaggacgggccttagtaccctatagaagcaatccttagaggacccggaagaacgcaatagacaaccgaaacgaggc
gttaggtactaaccgggtcgacgctcgttatcaacttggagcaacgaacggcaaacaagacccgaacggtccgaattaagct
tgacggaacgggttcccgttgataacgtccgtctcaatagttgcgtctagttggcgccttggtttacaaaggcgaaaggttg
ctatctttgccgggtacccttaattcggaacctccttggtaatcgcctctacccggattcgtagtagccctccttaatttgc
ccgccaactcttgctacctccttaaactcaagcggccgtccgaccaaaccaattagcaagcgctacgaacgtttgtaaggcg
ttgccgtcaagaaccgatgttgacggactaatcctgacatcatagtagttcagacgccgttaagcgccaattgagattggac
caagaaagcggaaggaggaatcgggtagttgtaagagccgaaagtaccgaagaagcccaagttggtcaagggctacaaggca
actagagtcttagccgggtcaaaggccaaacctcgacttgttggtagtctaacgggcaattgattggtaccggtcgctagtt
agggagcttcccaaattgaggctaccctaagcaaacgcggtaagctcttgggttgggacaatcggactttgggcaacgactt
agtatcgaccggatagtacgtaacgtcttggcttcgggagtaggttacgaaactacccggtagctactttcgtaggtttcgt
ctccggtccggtttcgtttcttgtattcccttggcggtcgttgccaaccaatcttggccaacccgtataaatccgctcgatt
tgacctttcggcctaagttgtacctaggacgttagaacgacctcgcgtagtaaaggacgggaaggtccaacaacgcttacgg
ttgagctcgttgcccttatagaacgcggttgttgttcccaattcaacctcgcctaacctttgtccggttcaacggaaatcgg
gacttacgggaacggttacccaagaaccgtccaaaccgataaaccgttagttacctcggccgagtcaattatccgaggacgg
actattccgggtcgctcttggaatacgccgaagttgttacggcggagttctagagtaaagcctaagggctaagtccctctac
cgaaccgacgatcctaaacgcgtttcggaaagttggcgaaactccaaacctctcaaagagccgtcgaagaggcaaaccaagt
agcttaaggtcggccaataggaccttcgctagtccaagggaaaccggagattaaggaccttctaacccgctaccaacaacaa
caaggtaccgaaacggaacccttccgtcaaacttgaacgcaaggttagggagggtccgattaacggcaacgattacttgctt
gtaaggcggagcgtctagggactctatagattgacgagcctccgcaaacaagagctattattaccggaccgggtttggtcct
caatcgacgtacggaggtaaggaattgcccttggagcgggaaaccgttagtttacccgtacgggtttcgttccttgcctagc
aaataaggtcgcggttatacgccgttgcttagaagccaacgaccaattgggtagggcgacgggacgatagtttgtccgttgt
tgctatcctctcgactatcccgttaacgggctaactataccgttgacggacgaaggatccgtaaagggccgttgttgactca
agcggcaatttccgaagcgcaagtaacgagaaggcgtcaacctacttgaaacccgaagaggagccgttgacaacccaaagaa
ggaattgccggcccaagcttcgttcgcttgttaacttgtagtccgacttggcgaccttgtatccgaataactcccgacgaag
gtcgttcgatactcgagttcggccgggattagttcctctcaaggaaagggcaacgaactagccaaagtacgcaaggtaccct
agaattggcaaggaacgggtttgctaccgacgcctaattggccgacaataggaggctaaacgttgttgccgtccgggtttat
tcgttccaaggctttggcctttattgcttcgctcgcgaacaatccgttctataacgcaacgcctattgggaaggcccggttt
gtttcgccaatttgccaagtcggtcctcttgatcgaacgctaactatcggagacgttcgtccgccttaaggaacgcttgtta
aggattccgcggaaggtatatccaagaaaggcccgacgtcttgcctaacccttatcctaccttccgacaattcctccttggc
ttgacgattacgagaagattcgagccgtccgaggtactaatctaaaccgggcgagtcgcaaagtttaacgccttaccctacc
cggaaatttcaaccaaccgaagcgcgtcgaaccttaaatctttccggcccttgctagtcggtacgttcctcctctcctttac
ctcttaccgcttaggctagtacgggacgttggaagtaaaggacgcgggcctacaaacgctattacccaagttggactccggg
caacttagaagggtcgttaaggcgctagagcggaagaaccaagttggatagcggacccaaccaagattattcgaggaaagcg
cctagtccgttcctccaaggcgaaatttggaggaccgacttgattcggagcaagaagttacaagggccgacaagacaattcg
ctcaacctcgaacgtcctaaccgtctctcttgctactctaccaaccgatagggctccctaatatcgagaggtttgcctctcg
tttcctcttaaccggaagggttacaaggcttgaaccgataacccaagagctaccggcgtaccgttccgaataaattaccggg
atcggacgtccttcgaattgtccaactccggcctatttgttggcctccgtaaacgacgaattgcgtccggattgactttccc
ggagatcaagtccaagacggcaatacgttgggcctcaacttaggtcgccgagctatctttcccgactaacttgattggttgc
gcgtcgatagtatccaattggactttcggcctagggacgttccgtataccaaggcttggacccaacttattgagcctaccgc
ttcgtaggctaaccttctaggccggcttaagacttaagaacgccgacgcgacaagtttacccttgcgctctctaaggttgcg
gtaagaggaattgagcctaaacccggcgctataacgacccaagtctaactacccaattccggcttgcgttgctcgcaatcct
aactaattgagccgaggaaccaagattggccgacccttccggtttgaaaccaaggagctaacgttccaacttggcccaaagt
tgaaactcgggagggccgtagggaagtagtatctaccgtacctagatacactcaatttgtactcggggttccgttttacatt
ccaggaaacgttgttgcgataggtattgaagcgagggtagaagggctattagttacgggcgaacgggatttggaccctcctt
tacttggcgctctagaatcaagggccgtaccttctttgtccttcggaaccttgcttgtcgtcaactcgctacttagatctcg
gcgacggaccaactataagtaagccctaaggcgtttgggtcgcaatataacggcttaggcctttcgccctttggacctacaa
gcaagagagagggatcaaacggttgggatttcggatccggacaattgtatccctaaccaacgacgacgttgttgcgcgttag
ggtcctttgaacgcgaagggaaaggattggcgaccttgggctaattactttcgcgcgtaacctacttcgaccttgagtacgc
cgaaattgctcggaccttctacgtatcgtaacaaccgctctacgacggccgtttcaaacaacgttggctaccgtagtccgta
ggtacgagtaggaggaagggaggttagggtaattcttccggacgaaatcaagtagcgttgggccttagtcccgattcgaagg
agctacttgtccgctacctccaacttagctccaacctaacccgggattggcaaataacgtaggccaaacgtcttgagttgac
ccaaacgtagttgggacggaacccttcggctttagaaggaattagcccgctacaaggccaaacaacgacgggcgtttacgta
gttgttccgttgacgttcaaagggacttggtcgccaatctactcgcggaaacaacttgcggtcggtaagtaagcgacggtaa
gaaagtcctcttccctagttcaagcggcctaattgaccaacgaacgtacaattcgacgctagggagatatagcgcctaggga
aagggcgggataattccgattccgtacggcaagttcgtcttgttcccgggcttcttagtttacggtcctacgcgttagagtc
gttgcgagtcggttccttattcggtttccgccaagagggtctataggactagtacaacccggtcggcccttctttgcttaaa
ggctctcctccttggcgcgtaactttgtcaagcgttgcgaagtaggtcaagaggaaaggcaacgctagcgtaactccgtcca
aacgttacttgtcggtttgacggagaaaccgtaacgacctccttgggtaagttcgatcttggctagcccttgctagtcgtct
ctcctcctttacgtaaggacaagcgtagcgctaactaggctacctctctccaaaccaaggaattgggtagcgaggtatccaa
cctctcaagtcggaataaggcccgttcccgtattatccaagacgtctaaccggactaccaacccgaactagggcggaattga
cgtaatattccgtcccgccttaatcgactcgtacgggatacggttaagtcttgctcgggtaatcttggcggttccctaactt
tcttcgccgtagaagtacgggtcgaaggcgagggatttgggtattggaggttacgctagccgattgtcaaggccgcttatag
attggcctacttaggctagattgcggaactcgaatcgacgtttcggtagccgaagaaagggaacgcgcctataagtacctcg
aaacccttcgttgagtagcctctcgaggataagcggttattgcgtcgggaagagttctagttacccgtacgccgtatcccta
tctttaacgggtaagccttgaccgcggtagatagtagggatcgtactcaaagggaagcgctcctcgttagaaaccgacgaag
cggcttgctatacaacgccgtactttaggcgatcccgtttcaaacccgcaaagcaagtctcgcttagcctaatctttggctt
ggccaacgtaacggctacttgggcgtctttactcgcctaaattgccggcctctctatctcaacgggagcaggaattgggtaa
cttgctaggcaaaccctagaggttcaacaaggtccgaccaacggagggtatcaagttgagtaggtaggcaaagaagggcgaa
cggttaacggaacaagagccggtaagcgtcttggtatcttcgttgcctcaacgtcggtctagttgactaagtaccgtcgagg
cgtttgcccttgagaacggttagaacctctcgtcgagtagaggtttaaggtcggaaagaaatcggtccgcggtatactctac
taccgaaaggtcgtcccggtagctagctaaacttggaaactcgggcccaatctccaagacaagtacaaggcctagcctcggc
ttaaaccaacctcggagagcttaggctacttcgtcaaggtaggctaatcgggcgttgattaaacgctacggtaccgaaagta
ccggacgtaacctactccgtttacgccctagtcgaaagggtaacgcttgggagttcgttgttgcgcccttacgtcaaagacg
gataaggagaagccctttcggtagttcgcctaagttgaactccgttgcctacgactaaccaaccaaggttgtccgtctctta
aggctcggaacttcaactcgggagacttgttcgaacgatattcgttgcgggttcggctttagaacgaaaccgaagggacgga
acgtttaaaccctcctccttctttccgccaaagtcgaaggactcgctttaacgcccaaataaccgccaaggatcggcgcgta
gtattagttcgtcttgccctatttgggaacttccctccgcaacctttacgctaccttacttggacgagagccaagtagagcc
aacggttacctcttagggcccggtttgtaatataaggcctcgctaaggaagagcgcgaaccgcttaaatctccttagcggcc
tcttcttattaccggcgactacgcggaagaggacaaattcccaactcgttcaattgggcccgactttggtccaacttagttg
gagtctagacctaggtattggcccggttaaccgggttagggttgtctagggagaatatcccgtgaagcttgagtggaatcgc
cgctgacgtgtgaggcgctagagcatagaccgaaaccaattggtttctagagcggtagggtagctagggttaacttacccgt
caacggccgttagcccgttaactacaaaccgttgggacctttgggcaacttgttgctacgactttaagctcgaacccttcga
gagagcaagtacctagctcggaagtaacggcctagatttactcctcgccgaagtaggaagatcccggtcgacgcgttgtttc
taggtttgggttgctccttgggacctttgagtaacgagttcgaaccggaccaatacgttgccgtacctctttaatagtcttg
accgcctcggttggcttagacggtcggttagcaatatccgtcctctagaagggtacccgaaacttgcgtagtaacgcctaag
gacgtcctagacctaaccgaaagaccctaggagcaaggtacctaaggtcctatagtccgagccaaaggtcgtagttgactta
gaggccgggatcaagagggaagggacggatcaaactacttgaaggccttcggccgaattgacctcaaatattcccgggaccg
ttgggaaccttgctttccgagtctccgctagtcgttgaacttgagggatcttctactccgggaaaccgtcgcaaaccgacaa
ggcaataggacttctaatccgtcctagggagccttcaagagtcgacgagtcttggtaattcttggacgtttacgcggtacga
gcgttcgcttatcaaggcctccgatactaacttaaagggctcaaccggttgtcctaatccggagtactaagtacgggcaaac
tcggtaggacccgttgaaagaacaaagcgacccgaaagacttgcgttattccggctttcctttctcaagtcaaaccgtccgc
ttctttggaggttgttgaaggcctccctcaactcctaaccaaacaagaccaaggagcgaggagagaaattgcgaagcgaccc
taaccgagaaacttgtacgggtcaatcgcccaaccaactattacccaaccgtcctcctacttggctacaagggttagcgtca
agatttggcgcttaggaaacctcttgctagagcaaagaagctcccggaagatccaaaggacggtagggttctagcttcaaat
cgcgcttgacctaataagccgacggagccctcggtaaagtcaattcgggtagcttagaagccgcaaggagattacccttcgt
tagtccctccttcccgattcgagaggattacaacggaattgggaccgaagtaggccggaagttacggagttcgtcgtttcaa
accctcccaaacttacgggacgtattgtagggacccgggattccaaccctaattgttactcccggcgttgagttagagcctt
agacaaggtcgccaaacggaccctaactaagacgagttagcccgccaactaataccctagtaacccttcggtaaggtcgccg
actccgatatcaaacgcgttagaaaggcgacggcaattggtacggtctacgttgtcttaggacggttcgggaacaacgagga
aagacaataggcgctaagccgttgagtcccaacgtaagagaggagcaagacttgaaggcttggtcgggtctaaggcaaaccc
tttagcgattgcccgaggaacggtagttgatccgtaggttggagtctccgattaatccgttccggtttctccctcggttcgt
cgagataagggcgattagaggtttagcgccgtaagctaatccgaacgactacttccaattgctcggcggttacaagcttgct
cgttacgacggcaagatagtcgagggtttacgtataccgggagctacgccttcttggtttccaaatccggtcgctaagggta
cggtttaatccggtagccgacttggtaaccttattcgggaacgacaagcgcaagcgatcccaaacaaaccgtaactcgggta
atcaacaagagccgtccgctttacctcgttgtaagtcgggttagggcccaatctaccaaccaagggttaacctcttctattg
gagacggccgataagctttgcggccctcctaaccaattgagtacgttgcgttcttcccgcccttatacgtacctttcccttc
ttgtacgccgaccgttgacgaagtacctctagcgctagctatccgaattgttacgggctataggtcgtaaccgggatcgaag
ctcctaattccaagaccctttggcaacgattctaccaacggacgaggagctacgttcttacaagaacgcgcgaggaaggtta
ggccttaaacaaggttcggtacgattcccgggattctagcggtttagctccaaggcccttagagggtcctttaacgtttacc
aatccggacgtcggacaactattgcgattggacccaaccaactcttggcaattggaaactaagggccgacaataggcctacg
ggtagtaggagtaagacggtttcgttggcggtaactagacgcgagttgacaacccaaacaaggtagtctacgaagcggactc
ttcttccggagtcccgactagattacgaaggacaaagtcgcaagcctcaagccttgggttggagttctaaggagcccgtata
aacgcaaaggaggaaacgacttgaagccgaggaaaggcggaagtaatacttgaccaaccgcaagtcgctctattgtcgctag
ctagaaggtcctaccgagggcgtttgaattagacgtttggcggtcaagcaaacgggacgggagaaacctcttgcgttctcta
gtacttccttgggagtcgaacgaacaagcttgtagaacctacggtagcggccttagggtattacgttgttgacgaagggcgg
tactcccttgttaacttgaaggcccggttggcttctaccaatcaagggtacgttgggtcttgacggctaggttaatcgcgct
ccaattggcgcgtcctaatatagctctattgggaggacggttacgtattcggcctaaggttgggaccttgaacaaggtatgg
cacgcctaatctggacacaggagaggtctttcatgcgtatagtcacatccgaaggccgggagaagcaattggagttgaccct
atctccgcaagacctttgcgcaaccttgtaaggacgttgaaacggttgtacccaaacaacggccggttgatctaagggtaat
ccaagggcttacgggagcaacaagctagttcggactagagggtcctctagcgataaagctaacgttcgagaacgccgccgct
aattgttgttgtcaagctaccgaggcttcgcaatacgatcgaagctagtccgacggctcaaaggaaagccgtaatcgtcgcc
caactaccaatcgaaccttgaggtacccaagaggtcaataccctatcctaggcgaacgaaggatacaagggacccaatagca
aggttggatagtcgcccttggtattgggccaagaagaggcgatatcgtaaccgttgatccgttgagttacttcgaccgctct
aggttcttacggtcggtactcgcctaaagcggcttgtttcgaccttaaaccgaacgggcaagacgaattgaggaacaaggct
cccaaccaaggttgaacgttagcggcaattgatccaaccgtcgtaaccttgttgtagggtccgtttaaccgctaagcctaag
gaagccgagtcctaaataaaccaagccgcggactaggcaacgggttagttgtccgataattaccgagcgaccggaccgcaaa
tacaagttagtagaggcggacgtctaacgttgctcgaattggatcgactcgcaagaccctagttgggacttcccttgggatt
acctcttcgccttctagcaaggttgttgcggacggtagttaggtcgacttggtcgttcttgaccttcttcccgtccctaagc
tagggtaaccaaaggttcgcgattcgtccgggtaaatttgcgtagcgcgttgcttctaataaccggatcctccggataccca
atcgttggttaactctcgtccgattaggcgtcggcgtttacgttaagggttagaagaggcgggcaaaggaacttaacaacgt
tacccggaaagcgaccaaaccgaagtcgatcctcttgacgagaggagaaggaatttccaaagagcgggcttcaaacggctct
agcggcttcgtctaaaccttcgcaaccaacttcccaagacgacttggaacgcgtagagtaaggccgctttgttgttgggatc
ttcttccaatccggatatccggtcctaccgaagaaggtattggtaagggatcgcctccgttgaccgattacttacttgatcg
cggccgttagcaagtccggtatacgcctcaatagtttacgcaagcggaggaagcttggttgggtaaccgaagggaagctccc
tacttagacgggtttggagtttacgtccaaacgctcgcctcttattaccggaagccctcgatctttggaccgcgtaggacaa
ctacctacgtcgctcttaaattcgaccgacaacgttcgtaaggcctacggcttccttggttgttacggcgttcaatagcaag
cgttagcgaaggccgggagtaataataggagggagtcaagagagattatcggtcggagccaagttgagtcggacccaagtta
aaccgtcgtcgtacggttaatcctctaaccggtaaacctccgttcgttgggtcgatccaaacctttctctcctaccgaccgc
ttaagcctagtcgaggtaccaagctaagtagtcaacggacgtaacttcccgtcgttacgcttggactacttcttaagcggcg
acaaggattggacgtccaagtacccaatctcgagtagctcaattaccgggccaactacccaagactaaggcgggttgttcga
gctaaccaaggactcccgacgtaattgtacccgccgatcgtaaattcgcccaataagcggattgggtttgtcggactccgac
gttaacgaacccaataagggatagttaagggcccgacgaagttaaagccggacggctaaggtacaattgttacggcccggtt
cctttgtccgtttctacctcaaccttgccgcgaacgactcgtttaaccgaaggctagaaactttggcgaaacggtacttacg
agggacctagctttgtaagcggttatccgcgcggacttatttggaggtcgttgggcctaacctcttggttgtagacccgata
ccgtcggcttcaattggaagatcgaaggctccttagtatcgggaagggtagttcctacgcgttaggaaaggctcttaaacgc
ccttgagcaagccgtcaacgtttagtcgttgttggacccgaggttgtaggtaagtaggatacgactcgggtccgcgaagaaa
tataggtcggcaacttagagtcccgagctttagggaacaacaaacctcggctattcctacgcggaaacgctacctaaagaag
ggcaacgacaagcgcgaacggtataacgggtacgactactcccgacttaggttaccttaacgggcaaccgaacgcttagctc
aaacgttcggtcccaaccttcaattgtcgggtccctctagggttacaatttgctccgcctctagggaggtttcgctatagca
agttcgttggtaggcttgacgggtttggttgcgaggcaatataggaggtattgggtctttaggacgagcgagtttggccgca
atcgaatttgacgcctagggcgaaccggtttaaccgaagcaactattggttgggttcaagggttgggtctaccggaaatcga
gttgggtcgtactccggagttctcaagccaacttaggcaactcaaacgattcgtccgctaactccgacttggttgtcgtata
agcgctctcctttggagtctttaacgtccgggtttacgagactacgcgattgcctcctaaagcctttaaccggtagccgggc
ttaagcccgaaagagctttgaaacgcggatatccgcttgttgtcaattgtcgaccggccttagctaagggacggagggctag
tttatacgggtcggagattcaatatgtgtcgtctatcctcagtcggtaactgcgcatagttggctctataggttcccggaac
ttcaatcgttccgctcctttaggttgaaggcgtttacggcgggtaaccgtttagggttagcaaggcaacctcgaggaaatag
gtcgggtattgagggaagtactagaagcttcccggagggttagaaggagggtaacggttaacgggacgaggaccaatttgtc
ggttgtcaacggttagttcgggtcaaaggaaaggacggatactttgctcggccgacctagaagttgagaagagaggtccgga
ggttgggtttcaattaggttgccgtttagggcctagggtaacaaggtttggtttcgagccgcttgactacggatttgaccaa
accaagcgtttaaggtcacatcgcatgaat

1-15. (canceled)

16. A device for the storage and/or the editing of digital data comprising at least one double stranded, replicative, composite nucleic acid molecule comprising a nucleic acid of formula (I):
5′-([UP]-[DB]-[DO])ₓ-3′ (I),
wherein,
[DB] represents a digital data-encoding nucleic acid having a length of from about 8 nucleotides to about 10⁶ nucleotides, optionally from about 500 nucleotides to about 5,000 nucleotides;
[UP] and [DO] represent a pair of non-digital data-encoding nucleic acids, each having a length of from about 0 nucleotide to about 10⁴ nucleotides, optionally from about 10 nucleotides to about 200 nucleotides; and
x represents 1 to about 10⁵.

17. The device according to claim 16, wherein the composite nucleic acid molecule has a length of from about 500 nucleotides to about 10¹¹ nucleotides, optionally from about 10³ nucleotides to about 10⁵ nucleotides.

18. The device according to claim 16, wherein the nucleic acid of formula (I) has a C+G percentage of from about 35% to about 65%.

19. The device according to claim 16, wherein the nucleic acid of formula (I) does not encode one or more RNA(s), optionally does not encode one or more mRNA(s).

20. The device according to claim 16, wherein the nucleic acid of formula (I) does not comprise one or more initiation codon(s) and/or comprises one or more stop codon(s) per about 200 nucleotides in all 6 reading frames.

21. The device according to claim 16, wherein the nucleic acid of formula (I) does not comprise one or more restriction site(s) for the enzymes or isoschizomers thereof selected in the group consisting of BamHI, BsaI, BbsI, EcoRI, FokI and I-SceI.

22. The device according to claim 16, wherein the nucleic acid of formula (I) does not comprise one or more repeat(s) of at least 4 identical nucleotides.

23. The device according to claim 16, wherein each nucleotide of the [DB] nucleic acid encodes 1 or 2 bits of the digital data.

24. The device according to claim 16, wherein the [UP] and [DO] nucleic acids each contain at least one barcode-encoding nucleic acid and/or at least one metadata-encoding nucleic acid.

25. A method for storing digital data comprising the steps of:
a) assigning to said digital data at least one double stranded digital data-encoding [DB] nucleic acid sequence (S_{DB}) and at least one pair of non-digital-data-encoding [UP] and [DO] nucleic acid sequences (S_{UP}) and (S_{DO});
b) synthesizing the at least one nucleic acid of formula (Ia):
5′-([UP]-[DB]-[DO])-3′ (Ia),
from the sequences (SᵤP), (S_{DB}) and (S_{DO}), respectively;
c) assembling the one or more nucleic acid(s) of formula (Ia) so as to obtain a double stranded, replicative, composite nucleic acid molecule comprising a nucleic acid of formula (I):
5′-([UP]-[DB]-[DO])ₓ-3′ (I),
wherein x represents 1 to about 10⁵;
d) storing at least one pool comprising from 1 to about 10⁹ composite nucleic acid molecule(s) of distinct sequence and comprising a nucleic acid of formula (I) obtained at step c) into a storage cell.

26. The method according to claim 25, further comprising the step of:
e) organizing and grouping the pools obtained at step d) into at least one array comprising from 1 pool to about 10⁶ pools, preferably about 96 or about 384 pools.

27. The method according to claim 25, wherein the composite nucleic acid molecule obtained at step c) is a plasmid, a cosmid, a prokaryotic chromosome or a eukaryotic chromosome.

28. The method according to claim 25, wherein it further comprises the steps of:
c1) amplifying in vivo the at least one composite nucleic acid molecule comprising a nucleic acid of formula (I) obtained at step c); and
c2) extracting and purifying the amplified composite nucleic acid molecule obtained at step c1).

29. The method according to claim 28, wherein step c1) is performed in vivo by a living organism, optionally a microorganism.

30. A method for retrieving a digital data stored by a device according to claim 1, said method comprising the steps of:
a) sequencing at least one nucleic acid of formula (Ia) comprised in a double stranded, replicative, composite nucleic acid molecule comprising a nucleic acid of formula (I), so as to obtain at least one nucleic acid sequence (S_{UP}-S_{DB}-S_{DO});
b) converting the at least one nucleic acid sequence (S_{DB}) into digital data;
wherein step a) is optionally preceded by step a0) of amplifying the at least one nucleic acid of formula (Ia).

31. A method for retrieving a digital data stored by the method according to claim 25, said method comprising the steps of:
a) sequencing at least one nucleic acid of formula (Ia) comprised in a double stranded, replicative, composite nucleic acid molecule comprising a nucleic acid of formula (I), so as to obtain at least one nucleic acid sequence (S_{UP}-S_{DB}-S_{DO});
b) converting the at least one nucleic acid sequence (S_{DB}) into digital data;
wherein step a) is optionally preceded by step a0) of amplifying the at least one nucleic acid of formula (Ia).
